(19) United States
(12) Patent Application Publication
(10) Pub. No.: US 2004/0147584 A2
(43) Pub. Date: Jul. 29, 2004

(54) 1,3-DIARYLPROP-2-EN-1-ONES, COMPOSITIONS CONTAINING THEM AND USE THEREOF

(75) Inventors: Patrick Mailliet, Fontenay Sous Bois (FR); Cecile Combeau, Fontenay Aux Roses (FR); Marie-Christine Bissery, Vitry Sur Seine (FR); Mari-Pierre Cherrier, Ivry Sur Seine (FR); Thomas Caulfield, Paris (FR); Gilles Tiraboschi, Montgeron (FR)

Correspondence Address: FINNEGAN, HENDERSON, FARABOW, GARRETT & DUNNER, LLP, 1300 I STREET, NW, WASHINGTON, DC, 20005, US

(73) Assignee: Aventis Pharma S.A., Antony (FR)

(21) Appl. No.: 10/309,076

(22) Filed: Dec. 4, 2002

Related U.S. Application Data
(60) Provisional application No. 60/346,979, filed on Jan. 11, 2002.

(30) Foreign Application Priority Data
Nov. 14, 2002 (FR) 02 14217
Dec. 5, 2001 (FR) 01 15739

(51) Int. Cl. A61K 31/405; A61K 31/401; A61K 31/198; C07C 237/28

(52) U.S. Cl. 514/414; 514/423; 514/464; 514/419; 514/563; 548/454; 548/494; 548/537; 564/161; 564/163

(57) ABSTRACT

### Abstract of the Disclosure

1,3-Diarylprop-2-en-1-ones and derivatives, compositions containing them, manufacturing process and use. Substituted 1,3-diarylprop-2-en-1-ones with therapeutic activity may be used in oncology.

### Detailed Description of the Invention

### Cross Reference to Related Applications

The present application claims priority under 35 U.S.C. §119(e) of U.S. Provisional Application No. 60/346,979, filed January 11, 2002, the disclosure of which is expressly incorporated by reference herein. The present application also claims priority under 35 U.S.C. §119 of French Application Nos. 0115739 and 0214217, filed December 5, 2001 and November 14, 2002, respectively, the disclosures of which are expressly incorporated by reference herein. The present invention relates to chemical compounds, such as 1,3to compositions containing them and to their use as medicinal products. The invention also relates to specific 1,3-diarylprop-2-en-1-ones with anticancer activity, such as inhibitory activity on tubulin polymerization.

### Background of Invention

1,3-diarylprop-2-en-1-ones, or "chalcones", have been widely described in the literature for more than a century. However, although certain publications deal with therapeutic applications of chalcones, few of them mention their use in oncology.

Among the documents describing the use of chalcones in oncology, mention may be made of the following patents and publications:

WO 01/72980, discloses substituted chalcones with anticancer and anti-inflammatory activity. These chalcones are characterized in that they are 1derivatives in which the 3group is never substituted with an amino group.

WO 99/22728, especially claims, in general, substituted chalcones, for inhibiting the 5αactivity towards steroid hormones, for the purpose of treating pathologies such as alopecia, baldness, obesity, skin diseases, prostate cancer and breast cancer. No specific example of a chalcone is presented in the description, only the structure of the reference product HZIV(E-3-(4-N,N-dimethylaminophenyl)-1-(3,4,5-trimethoxyphenyl)prop-2-en-1one) is presented in Figure 2.

WO 99/00114, claims the use of chalcones in which the prop-2-en-1-one chain may be saturated or unsaturated. The preparation examples are limited to certain families of chalcones. For each of the examples presented, one of the two phenyl nuclei is monoWhen an amino group is present, it is in the N,N-dimethylamino form and it is the only substituent of one of the two phenyl nuclei borne by the propenone chain.

Two products are cited as having anticancer activity. These are 1-(4-hydroxyphenyl)-3-(2,3-diimethoxyphenyl)-prop-2-en-1-one and 1-(4-hydroxyphenyl)-3-(2,5-dimethoxyphenyl)-prop-2-en-1-one.

WO 98/58913, presents chalcones derived from 1-(2-hydroxyphenyl)-3-arylprop-2-en-1-one with antiproliferative activity.

EP 288 794-B1, claims the use in oncology of 1-(aryl)-3-(4-X-phenyl)prop-2-en-1-ones in which X represents a substituent NR₂ or NHCOR, where R=alkyl.

WO 91/17749, claims a method for treating cancer especially using chalcones. These chalcones are described and claimed in very general terms. Thus, any substituents can replace any hydrogen, whether said hydrogen is on the prop-2-en-1-one chain or on a phenyl nucleus. None of the chalcones described bear amino groups on either of the aryl groups.

Michael L.et al., in the article published in J. Med. Chem. 1990, vol. 33, pp. 1948present chalcones that may be used as antimitotic agents. Chalcones in which the phenyl in position 3 on the prop-2-en-1-one chain is substituted either (i) in position 4 with NHC(O)CH₃, C(CH₃)₃, SCH₃, S(O)CH₃, N(CH₃)₂, NH₂, NO₂, F, CN, OCH(CH₃)₂, Br, CF₃, NNH-butyl, O-butyl, NHC(O)OCH₃, O-butyl, or N(C₂H₅)₂, or (ii) in position 3 with NHC(O)CH₃, N(CH₃)₂, NH₂ or NO₂, are presented and tested in vitro on cancer cell lines. None of the chalcones bears another group in addition to the amino group on one of the aryl nuclei.

Sylvie Ducki et al., in the article published in Bioorg. Med. Chem. Letters 1988, vol. 8 pp 1051present chalcones with antimitotic activity. Their study is based on the work by Michael L. Edwards et al. cited above. The authors observed that the replacement of a 4substituent with 4and 3substituents considerably improves the antimitotic activity, especially with respect to K562 cells.

### Detailed Description

Now, it has been found, surprisingly, that products containing the 1,3-diphenyl-prop-2-en-1-one unit in which the phenyl in position 3 is substituted with two different groups, at least one of which is an amine or an amine precursor, have large inhibitory activity on tubulin polymerization.

Furthermore, these products tend to very strongly induce necrosis in vivo, which is a highly favorable result with regard to the subsequent development of medicinal products that are effective for treating cancers.

Next, it has been observed that, with the products of the invention, the tumor necrosis survives in the minutes following the injection of the test product, and that the core of the tumor is totally destroyed within a day, with no apparent effect on the neighboring healthy cells. These products might consequently be useful for treating patients suffering from inoperable tumors, that is to say tumors whose surgical removal presents a very major risk (i) to the immediate survival of the patient, or (ii) to the possible consequences on his quality of life (invalidation).

Finally, the products of the invention are generally rapidly metabolized by the body, which limits their long-term effect.

These products correspond to formula (I) below:

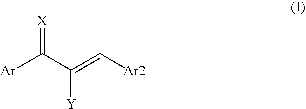

in which

a) Y is selected from the group consisting of halogen, C₁ ₇ linear alkyl, C₁ ₇ branched alkyl, substituted C₁ ₇ linear alkyl, substituted C₁ ₇ branched alkyl, cycloalkyl, substituted cycloalkyl, NH₂, NH(R4), N(R4)₂, aralkyl, substituted aralkyl, COOH, COO(R4), CONH₂, CONH(R4), CON(R4)₂, CN, in which R4 represents an optionally substituted C₁ ₇ alkyl or cycloalkyl group and, when two radicals R4 are present, they may be linked together to form a ring;

b) Ar2 is selected from the group consisting of:

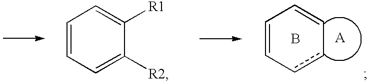

in which:

1)when Ar2 is

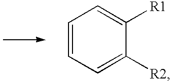

then one of the radicals R1 and R2 is selected from the group consisting of NH₂, NH₂·HZ, NHC(O)-amino acid, NH-(GP); N=(GP); in which the amino acid may be serine; in which GP is a metabolizable substituent allowing the functional group to be changed:

NH-(GP) →NH₂orN=(GP) → NH₂

and in which HZ is an organic or mineral acid; and the other radical R1 or R2 is selected from the group consisting of CH₃, C₂H₅, OCH₃, OC₂H₅, SCH₃, NH(R5), N(R5)₂, N(R5)(GP), N(R5)C(O)-amino acid, in which R5 represents a C₁-C₂ alkyl group and, when two radicals R5 are present, they may be linked together to form a ring;

2) when Ar2 is

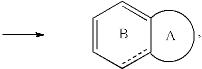

then A is a 5- or 6-membered heterocycle, fused to a benzene ring B, said heterocycle A is aromatic or non-aromatic, comprising one or two hetero atoms, at least one of which is a nitrogen atom linked directly to B and bearing a side chain R8, in which R8 is chosen from the group consisting of H, (C₁-C₃)alkyl, (C₁ ₃)alkyl-OH, (C₁-C₃)alkyl-O(C₁-C₃)alkyl, (C₁-C₃)alkyl-NH₂, (C₁-C₃)alkyl-NH(R7), (C₁ ₃)alkyl-N(R7)₂,

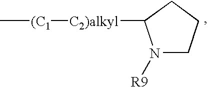

in which R9 is chosen from H, (C₁-C₃)alkyl, in which each R7 independently represents a (C₁-C₃)alkyl or (C₃-C₇) cycloalkyl group, or alternatively, when two radicals R7 are present, they are linked together to form a 5-membered heterocycle;

c) X is selected from the group consisting of O, NOH, NO(R3), in which R3 is selected from the group consisting of H, C₁-C₇ linear alkyl, C₁-C₇ branched alkyl, cycloalkyl, C₁-C₇ linear haloalkyl, C₁-C₇ branched haloalkyl, substituted cycloalkyl, halocycloalkyl, aralkyl, substituted aralkyl; and

d) Ar is selected from the group consisting of 2,5-dimethoxyphenyl, 2,3,4-trimethoxyphenyl, 3,4,5-trimethoxyphenyl, 2,3,5-trimethoxyphenyl, 2,4,5-trimethoxyphenyl, 2,3,4,5-tetramethoxyphenyl, 3-methoxy-4,5-methylenedioxyphenyl, 3-methoxy-4,5-ethylenedioxyphenyl, 2-methoxy-4,5-methylenedioxyphenyl, 2-methoxy-4,5-ethylenedioxyphenyl, 2-methoxy-3,4-methylenedioxyphenyl and 2-methoxy-3,4-ethylenedioxyphenyl radicals.

X may be oxygen.

Ar may be 3,4,5-trimethoxyphenyl or 3-methoxy-4,5-methylenedioxyphenyl.

Y may be selected from the group consisting of Cl, Br, CH₃ and CH₂CH₃.

A first product in accordance with the invention contains a substituent Ar2 such that Ar2 =

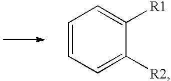

in which R1 and R2 are selected from the group of combinations (R1, R2) consisting of, respectively, (NH₂, OCH₃), (NH₂, OC₂H₅), (NH₂, N(R5)₂), (N(R5)₂, OCH₃), (N(R5)₂, OC₂H₅), (N(R5)₂, NH₂), (OCH₃, NH₂), (OC₂H₅, NH₂).

Another product in accordance with the invention contains a substituent Ar2 such that Ar2 =

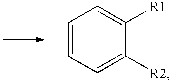

in which one of the radicals R1 and R2 is NHC(O)-amino acid, and in that the amino acid is selected from natural amino acids and unnatural amino acids. Amino acids may be chosen from the group consisting of glycine, N-methylproline, serine, lysine and N-ω-nitroarginine, and the amino acid is in enantiomerically pure or racemic form, or is enriched in one enantiomer.

The products in accordance with the invention are present in free or salified form. One salified form is a hydrochloride.

Yet another product in accordance with the invention contains a substituent Ar2 such that Ar2 =

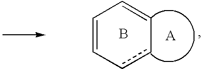

in which Ar2 is selected from the group consisting of

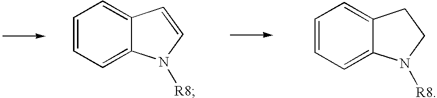

R8 may represent a methyl, hydroxymethyl or 2-dimethylaminoethyl group.

Still another product in accordance with the invention may be chosen from the following group:

E-3-(3-Amino-4-methoxyphenyl)-2-methyl-1-(3,4,5-trimethoxyphenyl)propenone;

E-3-(4-Amino-3-methoxyphenyl)-2-methyl-1-(3,4,5-trimethoxyphenyl)propenone;

E-3-(3-Amino-4-methoxyphenyl)-2-methyl-1-(2,5-dimethoxyphenyl)propenone hydrochloride;

E-3-(3-Amino-4-methoxyphenyl)-2-bromo-1-(3,4,5-trimethoxyphenyl)propenone;

E-2-Methyl-3-(1-methyl-1-H-indol-5-yl)-1-(3,4,5-trimethoxyphenyl)propenone; ;

E-2-Methyl-3-(1-methyl-2,3-dihydro-1-H-indol-5-yl)-1-(3,4,5-trimethoxyphenyl)propenone;

E-2-Methyl-3-(1-methyl-1-H-indol-5-yl)-1-(3,4,5-trimethoxyphenyl)propenoneoxime;

E-2-Methyl-3-[1-(2-dimethylaminoethyl)-1-H-indol-5-yl]-1-(3,4,5-trimethoxyphenyl)propenone hydrochloride;

E-2-Methyl-3-(1-hydroxymethyl-1-H-indol-5-yl)-1-(3,4,5-trimethoxyphenyl)propenone;

E-2-Methyl-3-(1-methyl-1-H-indol-5-yl)-1-(3-methoxy-4,5-methylenedioxyphenyl)propenone;

E-2-Methyl-3-[1-(2-dimethylaminoethyl)-1-H-indol-5-yl]-1-(3-methoxy-4,5-methylenedioxyphenyl)propenone;

(S)-2,6-Diaminohexanoic acid {2-methoxy-5-[2-methyl-3-oxo-3-(3,4,5-trimethoxyphenyl)propenyl]phenyl}amide dihydrochloride;

3-(3-[N-ω-nitro-L-arginineamido]-4-methoxyphenyl)-2-methyl-1-(3,4,5-trimethoxyphenyl)propenone;

1-Methylpyrrolidine-2-carboxylic acid {2-methoxy-5-[2-methyl-3-oxo-3-(3,4,5-trimethoxyphenyl)propenyl]}amide hydrochloride;

Aminoacetic acid {2-methoxy-5-[2-methyl-3-oxo-3-(3,4,5-trimethoxyphenyl)propenyl]phenyl}amide hydrochloride.

Another product in accordance with the invention is (S)-2-amino-3-hydroxypropanoic acid {2-methoxy-5-[2-methyl-3-oxo-3-(3,4,5-trimethoxy}amide hydrochloride.

The list of products below is also representative of the invention:

E-3-(3-Amino-4-methoxyphenyl)-2-chloro-1-(3,4,5-trimethoxyphenyl)propenone

E-3-(3-Amino-4-methoxyphenyl)-2-bromo-1-(3,4,5-trimethoxyphenyl)propenone

E-3-(3-Amino-4-methoxyphenyl)-2-methyl-1-(3,4,5-trimethoxyphenyl)propenone

E-3-(3-Amino-4-methoxyphenyl)-2-ethyl-1-(3,4,5-trimethoxyphenyl)propenone

E-3-(3-Amino-4-methoxyphenyl)-2-propyl-1-(3,4,5-trimethoxyphenyl)propenone

E-3-(3-Amino-4-methoxyphenyl)-2-(1-methylethyl)-1-(3,4,5-trimethoxyphenyl)propenone

E-3-(3-Amino-4-ethoxyphenyl)-2-chloro-1-(3,4,5-trimethoxyphenyl)propenone

E-3-(3-Amino-4-ethoxyphenyl)-2-bromo-1-(3,4,5-trimethoxyphenyl)propenone

E-3-(3-Amino-4-ethoxyphenyl)-2-methyl-1-(3,4,5-trimethoxyphenyl)propenone

E-3-(3-Amino-4-ethoxyphenyl)-2-ethyl-1-(3,4,5-trimethoxyphenyl)propenone

E-3-(3-Amino-4-ethoxyphenyl)-2-propyl-1-(3,4,5-trimethoxyphenyl)propenone

E-3-(3-Amino-4-ethoxyphenyl)-2-(1-methylethyl)-1-(3,4,5-trimethoxyphenyl)propenone

E-3-[3-Amino-4-(N,N-dimethylamino)phenyl]-2-chloro-1-(3,4,5-trimethoxyphenyl)propenone

E-3-[3-Amino-4-(N,N-dimethylamino)phenyl]-2-bromo-1-(3,4,5-trimethoxyphenyl)propenone

E-3-[3-Amino-4-(N,N-dimethylamino)phenyl]-2-methyl-1-(3,4,5-trimethoxyphenyl)propenone

E-3-[3-Amino-4-(N,N-dimethylamino)phenyl]-2-ethyl-1-(3,4,5-trimethoxyphenyl)propenone

E-3-[3-Amino-4-(N,N-dimethylamino)phenyl]-2-propyl-1-(3,4,5-trimethoxyphenyl)propenone

E-3-[3-Amino-4-(N,N-dimethylamino)phenyl]-2-(1-methylethyl)-1-(3,4,5-trimethoxyphenyl)propenone

E-3-[4-Amino-3-methoxyphenyl)-2-chloro-1-(3,4,5-trimethoxyphenyl)propenone

E-3-[4-Amino-3-methoxyphenyl)-2-bromo-1-(3,4,5-trimethoxyphenyl)propenone

E-3-[4-Amino-3-methoxyphenyl)-2-methyl-1-(3,4,5-trimethoxyphenyl)propenone

E-3-[4-Amino-3-methoxyphenyl)-2-ethyl-1-(3,4,5-trimethoxyphenyl)propenone

E-3-[4-Amino-3-methoxyphenyl)-2-propyl-1-(3,4,5-trimethoxyphenyl)propenone

E-3-[4-Amino-3-methoxyphenyl)-2-(1-methylethyl)-1-(3,4,5-trimethoxyphenyl)propenone

E-3-[4-Amino-3-ethoxyphenyl)-2-chloro-1-(3,4,5-trimethoxyphenyl)propenone

E-3-[4-Amino-3-ethoxyphenyl)-2-bromo-1-(3,4,5-trimethoxyphenyl)propenone

E-3-[4-Amino-3-ethoxyphenyl)-2-methyl-1-(3,4,5-trimethoxyphenyl)propenone

E-3-[4-Amino-3-ethoxyphenyl)-2-ethyl-1-(3,4,5-trimethoxyphenyl)propenone

E-3-[4-Amino-3-ethoxyphenyl)-2-propyl-1-(3,4,5-trimethoxyphenyl)propenone

E-3-[4-Amino-3-ethoxyphenyl)-2-(1-methylethyl)-1-(3,4,5-trimethoxyphenyl)propenone

E-3-[4-Amino-3-(N,N-dimethylamino)phenyl]-2-chloro-1-(3,4,5-trimethoxyphenyl)propenone

E-3-[4-Amino-3-(N,N-dimethylamino)phenyl]-2-bromo-1-(3,4,5-trimethoxyphenyl)propenone

E-3-[4-Amino-3-(N,N-dimethylamino)phenyl]-2-methyl-1-(3,4,5-trimethoxyphenyl)propenone

E-3-[4-Amino-3-(N,N-dimethylamino)phenyl]-2-ethyl-1-(3,4,5-trimethoxyphenyl)propenone

E-3-[4-Amino-3-(N,N-dimethylamino)phenyl]-2-propyl-1-(3,4,5-trimethoxyphenyl)propenone

E-3-[4-Amino-3-(N,N-dimethylamino)phenyl]-2-(1-methylethyl)-1-(3,4,5-trimethoxyphenyl)propenone

E-3-[3-(N,N-Dimethylamino)-4-methoxyphenyl]-2-chloro-1-(3,4,5-trimethoxyphenyl)propenone

E-3-[3-(N,N-Dimethylamino)-4-methoxyphenyl]-2-bromo-1-(3,4,5-trimethoxyphenyl)propenone

E-3-[3-(N,N-Dimethylamino)-4-methoxyphenyl]-2-methyl-1-(3,4,5-trimethoxyphenyl)propenone

E-3-[3-(N,N-Dimethylamino)-4-methoxyphenyl]-2-ethyl-1-(3,4,5-trimethoxyphenyl)propenone

E-3-[4-Amino-3-(N,N-dimethylamino)-4-methoxyphenyl]-2-propyl-1-(3,4,5-trimethoxyphenyl)propenone

E-3-[4-Amino-3-(N,N-dimethylamino)-4-methoxyphenyl]-2-(1-methylethyl)-1-(3,4,5-trimethoxyphenyl)propenone

E-3-[3-(N,N-Dimethylamino)-4-ethoxyphenyl]-2-chloro-1-(3,4,5-trimethoxyphenyl)propenone

E-3-[3-(N,N-Dimethylamino)-4-ethoxyphenyl]-2-bromo-1-(3,4,5-trimethoxyphenyl)propenone

E-3-[3-(N,N-Dimethylamino)-4-ethoxyphenyl]-2-methyl-1-(3,4,5-trimethoxyphenyl)propenone

E-3-[3-(N,N-Dimethylamino)-4-ethoxyphenyl]-2-ethyl-1-(3,4,5-trimethoxyphenyl)propenone

E-3-[4-Amino-3-(N,N-dimethylamino)-4-ethoxyphenyl]-2-propyl-1-(3,4,5-trimethoxyphenyl)propenone

E-3-[4-Amino-3-(N,N-dimethylamino)-4-ethoxyphenyl]-2-(1-methylethyl)-1-(3,4,5-trimethoxyphenyl)propenone

E-3-(3-Amino-4-methoxyphenyl)-2-chloro-1-(2,5-dimethoxyphenyl)propenone

E-3-(3-Amino-4-methoxyphenyl)-2-bromo-1-(2,5-dimethoxyphenyl)propenone

E-3-(3-Amino-4-methoxyphenyl)-2-methyl-1-(2,5-dimethoxyphenyl)propenone hydrochloride

E-3-(3-Amino-4-methoxyphenyl)-2-methyl-1-(2,5-dimethoxyphenyl)propenone

E-3-(3-Amino-4-methoxyphenyl)-2-ethyl-1-(2,5-dimethoxyphenyl)propenone

E-3-(3-Amino-4-methoxyphenyl)-2-propyl-1-(2,5-dimethoxyphenyl)propenone

E-3-(3-Amino-4-methoxyphenyl)-2-(1-methylethyl)-1-(2,5-dimethoxyphenyl)propenone

E-3-(3-Amino-4-ethoxyphenyl)-2-chloro-1-(2,5-dimethoxyphenyl)propenone

E-3-(3-Amino-4-ethoxyphenyl)-2-bromo-1-(2,5-dimethoxyphenyl)propenone

E-3-(3-Amino-4-ethoxyphenyl)-2-methyl-1-(2,5-dimethoxyphenyl)propenone

E-3-(3-Amino-4-ethoxyphenyl)-2-ethyl-1-(2,5-dimethoxyphenyl)propenone

E-3-(3-Amino-4-ethoxyphenyl)-2-propyl-1-(2,5-dimethoxyphenyl)propenone

E-3-(3-Amino-4-ethoxyphenyl)-2-(1-methylethyl)-1-(2,5-dimethoxyphenyl)propenone

E-3-[3-Amino-4-(N,N-dimethylamino)phenyl]-2-chloro-1-(2,5-dimethoxyphenyl)propenone

E-3-[3-Amino-4-(N,N-dimethylamino)phenyl]-2-bromo-1-(2,5-dimethoxyphenyl)propenone

E-3-[3-Amino-4-(N,N-dimethylamino)phenyl]-2-methyl-1-(2,5-dimethoxyphenyl)propenone

E-3-[3-Amino-4-(N,N-dimethylamino)phenyl]-2-ethyl-1-(2,5-dimethoxyphenyl)propenone

E-3-[3-Amino-4-(N,N-dimethylamino)phenyl]-2-propyl-1-(2,5-dimethoxyphenyl)propenone

E-3-[3-Amino-4-(N,N-dimethylamino)phenyl]-2-(1-methylethyl)-1-(2,5-dimethoxyphenyl)propenone

E-3-(4-Amino-3-methoxyphenyl)-2-chloro-1-(2,5-dimethoxyphenyl)propenone

E-3-(4-Amino-3-methoxyphenyl)-2-bromo-1-(2,5-dimethoxyphenyl)propenone

E-3-(4-Amino-3-methoxyphenyl)-2-methyl-1-(2,5-dimethoxyphenyl)propenone

E-3-(4-Amino-3-methoxyphenyl)-2-ethyl-1-(2,5-dimethoxyphenyl)propenone

E-3-(4-Amino-3-methoxyphenyl)-2-propyl-1-(2,5-dimethoxyphenyl)propenone

E-3-(4-Amino-3-methoxyphenyl)-2-(1-methylethyl)-1-(2,5-dimethoxyphenyl)propenone

E-3-(4-Amino-3-ethoxyphenyl)-2-chloro-1-(2,5-dimethoxyphenyl)propenone

E-3-(4-Amino-3-ethoxyphenyl)-2-bromo-1-(2,5-dimethoxyphenyl)propenone

E-3-(4-Amino-3-ethoxyphenyl)-2-methyl-1-(2,5-dimethoxyphenyl)propenone

E-3-(4-Amino-3-ethoxyphenyl)-2-ethyl-1-(2,5-dimethoxyphenyl)propenone

E-3-(4-Amino-3-ethoxyphenyl)-2-propyl-1-(2,5-dimethoxyphenyl)propenone

E-3-(4-Amino-3-ethoxyphenyl)-2-(1-methylethyl)-1-(2,5-dimethoxyphenyl)propenone

E-3-[4-Amino-3-(N,N-dimethylamino)phenyl]-2-chloro-1-(2,5-dimethoxyphenyl)propenone

E-3-[4-Amino-3-(N,N-dimethylamino)phenyl]-2-bromo-1-(2,5-dimethoxyphenyl)propenone

E-3-[4-Amino-3-(N,N-dimethylamino)phenyl]-2-methyl-1-(2,5-dimethoxyphenyl)propenone

E-3-[4-Amino-3-(N,N-dimethylamino)phenyl]-2-ethyl-1-(2,5-dimethoxyphenyl)propenone

E-3-[4-Amino-3-(N,N-dimethylamino)phenyl]-2-propyl-1-(2,5-dimethoxyphenyl)propenone

E-3-[4-Amino-3-(N,N-dimethylamino)phenyl]-2-(1-methylethyl)-1-(2,5-dimethoxyphenyl)propenone

E-3-[3-(N,N-Dimethylamino)-4-methoxyphenyl]-2-chloro-1-(2,5-dimethoxyphenyl)propenone

E-3-[3-(N,N-Dimethylamino)-4-methoxyphenyl]-2-bromo-1-(2,5-dimethoxyphenyl)propenone

E-3-[3-(N,N-Dimethylamino)-4-methoxyphenyl]-2-methyl-1-(2,5-dimethoxyphenyl)propenone

E-3-[3-(N,N-Dimethylamino)-4-methoxyphenyl]-2-ethyl-1-(2,5-dimethoxyphenyl)propenone

E-3-[4-Amino-3-(N,N-dimethylamino)-4-methoxyphenyl]-2-propyl-1-(2,5-dimethoxyphenyl)propenone

E-3-[4-Amino-3-(N,N-dimethylamino)-4-methoxyphenyl]-2-(1-methylethyl)-1-(2,5-dimethoxyphenyl)propenone

E-3-[3-(N,N-Dimethylamino)-4-ethoxyphenyl]-2-chloro-1-(2,5-dimethoxyphenyl)propenone

E-3-[3-(N,N-Dimethylamino)-4-ethoxyphenyl]-2-bromo-1-(2,5-dimethoxyphenyl)propenone

E-3-[3-(N,N-Dimethylamino)-4-ethoxyphenyl]-2-methyl-1-(2,5-dimethoxyphenyl)propenone

E-3-[3-(N,N-Dimethylamino)-4-ethoxyphenyl]-2-ethyl-1-(2,5-dimethoxyphenyl)propenone

E-3-[4-Amino-3-(N,N-dimethylamino)-4-ethoxyphenyl]-2-propyl-1-(2,5-dimethoxyphenyl)propenone

E-3-[4-Amino-3-(N,N-dimethylamino)-4-ethoxyphenyl]-2-(1-methylethyl)-1-(2,5-dimethoxyphenyl)propenone

E-3-(3-Amino-4-methoxyphenyl)-2-chloro-1-(2,3,4-trimethoxyphenyl)propenone

E-3-(3-Amino-4-methoxyphenyl)-2-bromo-1-(2,3,4-trimethoxyphenyl)propenone

E-3-(3-Amino-4-methoxyphenyl)-2-methyl-1-(2,3,4-trimethoxyphenyl)propenone

E-3-(3-Amino-4-methoxyphenyl)-2-ethyl-1-(2,3,4-trimethoxyphenyl)propenone

E-3-(3-Amino-4-methoxyphenyl)-2-propyl-1-(2,3,4-trimethoxyphenyl)propenone

E-3-(3-Amino-4-methoxyphenyl)-2-(1-methylethyl)-1-(2,3,4-trimethoxyphenyl)propenone

E-3-(3-Amino-4-ethoxyphenyl)-2-chloro-1-(2,3,4-trimethoxyphenyl)propenone

E-3-(3-Amino-4-ethoxyphenyl)-2-bromo-1-(2,3,4-trimethoxyphenyl)propenone

E-3-(3-Amino-4-ethoxyphenyl)-2-methyl-1-(2,3,4-trimethoxyphenyl)propenone

E-3-(3-Amino-4-ethoxyphenyl)-2-ethyl-1-(2,3,4-trimethoxyphenyl)propenone

E-3-(3-Amino-4-ethoxyphenyl)-2-propyl-1-(2,3,4-trimethoxyphenyl)propenone

E-3-(3-Amino-4-ethoxyphenyl)-2-(1-methylethyl)-1-(2,3,4-trimethoxyphenyl)propenone

E-3-[3-Amino-4-(N,N-dimethylamino)phenyl]-2-chloro-1-(2,3,4-trimethoxyphenyl)propenone

E-3-[3-Amino-4-(N,N-dimethylamino)phenyl]-2-bromo-1-(2,3,4-trimethoxyphenyl)propenone

E-3-[3-Amino-4-(N,N-dimethylamino)phenyl]-2-methyl-1-(2,3,4-trimethoxyphenyl)propenone

E-3-[3-Amino-4-(N,N-dimethylamino)phenyl]-2-ethyl-1-(2,3,4-trimethoxyphenyl)propenone

E-3-[3-Amino-4-(N,N-dimethylamino)phenyl]-2-propyl-1-(2,3,4-trimethoxyphenyl)propenone

E-3-[3-Amino-4-(N,N-dimethylamino)phenyl]-2-(1-methylethyl)-1-(2,3,4-trimethoxyphenyl)propenone

E-3-(4-Amino-3-methoxyphenyl)-2-chloro-1-(2,3,4-trimethoxyphenyl)propenone

E-3-(4-Amino-3-methoxyphenyl)-2-bromo-1-(2,3,4-trimethoxyphenyl)propenone

E-3-(4-Amino-3-methoxyphenyl)-2-methyl-1-(2,3,4-trimethoxyphenyl)propenone

E-3-(4-Amino-3-methoxyphenyl)-2-ethyl-1-(2,3,4-trimethoxyphenyl)propenone

E-3-(4-Amino-3-methoxyphenyl)-2-propyl-1-(2,3,4-trimethoxyphenyl)propenone

E-3-(4-Amino-3-methoxyphenyl)-2-(1-methylethyl)-1-(2,3,4-trimethoxyphenyl)propenone

E-3-(4-Amino-3-ethoxyphenyl)-2-chloro-1-(2,3,4-trimethoxyphenyl)propenone

E-3-(4-Amino-3-ethoxyphenyl)-2-bromo-1-(2,3,4-trimethoxyphenyl)propenone

E-3-(4-Amino-3-ethoxyphenyl)-2-methyl-1-(2,3,4-trimethoxyphenyl)propenone

E-3-(4-Amino-3-ethoxyphenyl)-2-ethyl-1-(2,3,4-trimethoxyphenyl)propenone

E-3-(4-Amino-3-ethoxyphenyl)-2-propyl-1-(2,3,4-trimethoxyphenyl)propenone

E-3-(4-Amino-3-ethoxyphenyl)-2-(1-methylethyl)-1-(2,3,4-trimethoxyphenyl)propenone

E-3-[4-Amino-3-(N,N-dimethylamino)phenyl]-2-chloro-1-(2,3,4-trimethoxyphenyl)propenone

E-3-[4-Amino-3-(N,N-dimethylamino)phenyl]-2-bromo-1-(2,3,4-trimethoxyphenyl)propenone

E-3-[4-Amino-3-(N,N-dimethylamino)phenyl]-2-methyl-1-(2,3,4-trimethoxyphenyl)propenone

E-3-[4-Amino-3-(N,N-dimethylamino)phenyl]-2-ethyl-1-(2,3,4-trimethoxyphenyl)propenone

E-3-[4-Amino-3-(N,N-dimethylamino)phenyl]-2-propyl-1-(2,3,4-trimethoxyphenyl)propenone

E-3-[4-Amino-3-(N,N-dimethylamino)phenyl]-2-(1-methylethyl)-1-(2,3,4-trimethoxyphenyl)propenone

E-3-[3-(N,N-Dimethylamino)-4-methoxyphenyl]-2-chloro-1-(2,3,4-trimethoxyphenyl)propenone

E-3-[3-(N,N-Dimethylamino)-4-methoxyphenyl]-2-bromo-1-(2,3,4-trimethoxyphenyl)propenone

E-3-[3-(N,N-Dimethylamino)-4-methoxyphenyl]-2-methyl-1-(2,3,4-trimethoxyphenyl)propenone

E-3-[3-(N,N-Dimethylamino)-4-methoxyphenyl]-2-ethyl-1-(2,3,4-trimethoxyphenyl)propenone

E-3-[4-Amino-3-(N,N-dimethylamino)-4-methoxyphenyl]-2-propyl-1-(2,3,4-trimethoxyphenyl)propenone

E-3-[4-Amino-3-(N,N-dimethylamino)-4-methoxyphenyl]-2-(1-methylethyl)-1-(2,3,4-trimethoxyphenyl)propenone

E-3-[3-(N,N-Dimethylamino)-4-ethoxyphenyl]-2-chloro-1-(2,3,4-trimethoxyphenyl)propenone

E-3-[3-(N,N-Dimethylamino)-4-ethoxyphenyl]-2-bromo-1-(2,3,4-trimethoxyphenyl)propenone

E-3-[3-(N,N-Dimethylamino)-4-ethoxyphenyl]-2-methyl-1-(2,3,4-trimethoxyphenyl)propenone

E-3-[3-(N,N-Dimethylamino)-4-ethoxyphenyl]-2-ethyl-1-(2,3,4-trimethoxyphenyl)propenone

E-3-[4-Amino-3-(N,N-dimethylamino)-4-ethoxyphenyl]-2-propyl-1-(2,3,4-trimethoxyphenyl)propenone

E-3-[4-Amino-3-(N,N-dimethylamino)-4-ethoxyphenyl]-2-(1-methylethyl)-1-(2,3,4-trimethoxyphenyl)propenone

E-3-(3-Amino-4-methoxyphenyl)-2-chloro-1-(2,3,5-trimethoxyphenyl)propenone

E-3-(3-Amino-4-methoxyphenyl)-2-bromo-1-(2,3,5-trimethoxyphenyl)propenone

E-3-(3-Amino-4-methoxyphenyl)-2-methyl-1-(2,3,5-trimethoxyphenyl)propenone

E-3-(3-Amino-4-methoxyphenyl)-2-ethyl-1-(2,3,5-trimethoxyphenyl)propenone

E-3-(3-Amino-4-methoxyphenyl)-2-propyl-1-(2,3,5-trimethoxyphenyl)propenone

E-3-(3-Amino-4-methoxyphenyl)-2-(1-methylethyl)-1-(2,3,5-trimethoxyphenyl)propenone

E-3-(3-Amino-4-ethoxyphenyl)-2-chloro-1-(2,3,5-trimethoxyphenyl)propenone

E-3-(3-Amino-4-ethoxyphenyl)-2-bromo-1-(2,3,5-trimethoxyphenyl)propenone

E-3-(3-Amino-4-ethoxyphenyl)-2-methyl-1-(2,3,5-trimethoxyphenyl)propenone

E-3-(3-Amino-4-ethoxyphenyl)-2-ethyl-1-(2,3,5-trimethoxyphenyl)propenone

E-3-(3-Amino-4-ethoxyphenyl)-2-propyl-1-(2,3,5-trimethoxyphenyl)propenone

E-3-(3-Amino-4-ethoxyphenyl)-2-(1-methylethyl)-1-(2,3,5-trimethoxyphenyl)propenone

E-3-[3-Amino-4-(N,N,-dimethylamino)phenyl]-2-chloro-1-(2,3,5-trimethoxyphenyl)propenone

E-3-[3-Amino-4-(N,N,-dimethylamino)phenyl]-2-bromo-1-(2,3,5-trimethoxyphenyl)propenone

E-3-[3-Amino-4-(N,N,-dimethylamino)phenyl]-2-methyl-1-(2,3,5-trimethoxyphenyl)propenone

E-3-[3-Amino-4-(N,N,-dimethylamino)phenyl]-2-ethyl-1-(2,3,5-trimethoxyphenyl)propenone

E-3-[3-Amino-4-(N,N,-dimethylamino)phenyl]-2-propyl-1-(2,3,5-trimethoxyphenyl)propenone

E-3-[3-Amino-4-(N,N,-dimethylamino)phenyl]-2-(1-methylethyl)-1-(2,3,5-trimethoxyphenyl)propenone

E-3-[4-Amino-3-methoxyphenyl)-2-chloro-1-(2,3,5-trimethoxyphenyl)propenone

E-3-(4-Amino-3-methoxyphenyl)-2-bromo-1-(2,3,5-trimethoxyphenyl)propenone

E-3-(4-Amino-3-methoxyphenyl)-2-methyl-1-(2,3,5-trimethoxyphenyl)propenone

E-3-(4-Amino-3-methoxyphenyl)-2-ethyl-1-(2,3,5-trimethoxyphenyl)propenone

E-3-(4-Amino-3-methoxyphenyl)-2-propyl-1-(2,3,5-trimethoxyphenyl)propenone

E-3-(4-Amino-3-methoxyphenyl)-2-(1-methylethyl)-1-(2,3,5-trimethoxyphenyl)propenone

E-3-(4-Amino-3-ethoxyphenyl)-2-chloro-1-(2,3,5-trimethoxyphenyl)propenone

E-3-(4-Amino-3-ethoxyphenyl)-2-bromo-1-(2,3,5-trimethoxyphenyl)propenone

E-3-(4-Amino-3-ethoxyphenyl)-2-methyl-1-(2,3,5-trimethoxyphenyl)propenone

E-3-(4-Amino-3-ethoxyphenyl)-2-ethyl-1-(2,3,5-trimethoxyphenyl)propenone

E-3-(4-Amino-3-ethoxyphenyl)-2-propyl-1-(2,3,5-trimethoxyphenyl)propenone

E-3-(4-Amino-3-ethoxyphenyl)-2-(1-methylethyl)-1-(2,3,5-trimethoxyphenyl)propenone

E-3-[4-Amino-3-(N,N-dimethylamino)phenyl]-2-chloro-1-(2,3,5-trimethoxyphenyl)propenone

E-3-[4-Amino-3-(N,N-dimethylamino)phenyl]-2-bromo-1-(2,3,5-trimethoxyphenyl)propenone

E-3-[4-Amino-3-(N,N-dimethylamino)phenyl]-2-methyl-1-(2,3,5-trimethoxyphenyl)propenone

E-3-[4-Amino-3-(N,N-dimethylamino)phenyl]-2-ethyl-1-(2,3,5-trimethoxyphenyl)propenone

E-3-[4-Amino-3-(N,N-dimethylamino)phenyl]-2-propyl-1-(2,3,5-trimethoxyphenyl)propenone

E-3-[4-Amino-3-(N,N-dimethylamino)phenyl]-2-(1-methylethyl)-1-(2,3,5-trimethoxyphenyl)propenone

E-3-[3-(N,N-Dimethylamino)-4-methoxyphenyl]-2-chloro-1-(2,3,5-trimethoxyphenyl)propenone

E-3-[3-(N,N-Dimethylamino)-4-methoxyphenyl]-2-bromo-1-(2,3,5-trimethoxyphenyl)propenone

E-3-[3-(N,N-Dimethylamino)-4-methoxyphenyl]-2-methyl-1-(2,3,5-trimethoxyphenyl)propenone

E-3-[3-(N,N-Dimethylamino)-4-methoxyphenyl]-2-ethyl-1-(2,3,5-trimethoxyphenyl)propenone

E-3-[4-Amino-3-(N,N-dimethylamino)-4-methoxyphenyl]-2-propyl-1-(2,3,4-trimethoxyphenyl)propenone

E-3-[4-Amino-3-(N,N-dimethylamino)-4-methoxyphenyl]-2-(1-methylethyl)-1-(2,3,5-trimethoxyphenyl)propenone

E-3-[3-(N,N-Dimethylamino)-4-ethoxyphenyl]-2-chloro-1-(2,3,5-trimethoxyphenyl)propenone

E-3-[3-(N,N-Dimethylamino)-4-ethoxyphenyl]-2-bromo-1-(2,3,5-trimethoxyphenyl)propenone

E-3-[3-(N,N-Dimethylamino)-4-ethoxyphenyl]-2-methyl-1-(2,3,5-trimethoxyphenyl)propenone

E-3-[3-(N,N-Dimethylamino)-4-ethoxyphenyl]-2-ethyl-1-(2,3,5-trimethoxyphenyl)propenone

E-3-[4-Amino-3-(N,N-dimethylamino)-4-ethoxyphenyl]-2-propyl-1-(2,3,5-trimethoxyphenyl)propenone

E-3-[4-Amino-3-(N,N-dimethylamino)-4-ethoxyphenyl]-2-(1-methylethyl)-1-(2,3,5-trimethoxyphenyl)propenone

E-3-(3-Amino-4-methoxyphenyl]-2-chloro-1-(2,4,5-trimethoxyphenyl)propenone

E-3-(3-Amino-4-methoxyphenyl]-2-bromo-1-(2,4,5-trimethoxyphenyl)propenone

E-3-(3-Amino-4-methoxyphenyl]-2-methyl-1-(2,4,5-trimethoxyphenyl)propenone

E-3-(3-Amino-4-methoxyphenyl]-2-ethyl-1-(2,4,5-trimethoxyphenyl)propenone

E-3-(3-Amino-4-methoxyphenyl]-2-propyl-1-(2,4,5-trimethoxyphenyl)propenone

E-3-(3-Amino-4-methoxyphenyl]-2-(1-methylethyl)-1-(2,4,5-trimethoxyphenyl)propenone

E-3-(3-Amino-4-ethoxyphenyl]-2-chloro-1-(2,4,5-trimethoxyphenyl)propenone

E-3-(3-Amino-4-ethoxyphenyl]-2-bromo-1-(2,4,5-trimethoxyphenyl)propenone

E-3-(3-Amino-4-ethoxyphenyl]-2-methyl-1-(2,4,5-trimethoxyphenyl)propenone

E-3-(3-Amino-4-ethoxyphenyl]-2-ethyl-1-(2,4,5-trimethoxyphenyl)propenone

E-3-(3-Amino-4-ethoxyphenyl]-2-propyl-1-(2,4,5-trimethoxyphenyl)propenone

E-3-(3-Amino-4-ethoxyphenyl]-2-(1-methylethyl)-1-(2,4,5-trimethoxyphenyl)propenone

E-3-[3-Amino-4-(N,N-dimethylamino)phenyl]-2-chloro-1-(2,4,5-trimethoxyphenyl)propenone

E-3-[3-Amino-4-(N,N-dimethylamino)phenyl]-2-bromo-1-(2,4,5-trimethoxyphenyl)propenone

E-3-[3-Amino-4-(N,N-dimethylamino)phenyl]-2-methyl-1-(2,4,5-trimethoxyphenyl)propenone

E-3-[3-Amino-4-(N,N-dimethylamino)phenyl]-2-ethyl-1-(2,4,5-trimethoxyphenyl)propenone

E-3-[3-Amino-4-(N,N-dimethylamino)phenyl]-2-propyl-1-(2,4,5-trimethoxyphenyl)propenone

E-3-[3-Amino-4-(N,N-dimethylamino)phenyl]-2-(1-methylethyl)-1-(2,4,5-trimethoxyphenyl)propenone

E-3-[4-Amino-3-methoxyphenyl)-2-chloro-1-(2,4,5-trimethoxyphenyl)propenone

E-3-[4-Amino-3-methoxyphenyl)-2-bromo-1-(2,4,5-trimethoxyphenyl)propenone

E-3-[4-Amino-3-methoxyphenyl)-2-methyl-1-(2,4,5-trimethoxyphenyl)propenone

E-3-[4-Amino-3-methoxyphenyl)-2-ethyl-1-(2,4,5-trimethoxyphenyl)propenone

E-3-[4-Amino-3-methoxyphenyl)-2-propyl-1-(2,4,5-trimethoxyphenyl)propenone

E-3-[4-Amino-3-methoxyphenyl)-2-(1-methylethyl)-1-(2,4,5-trimethoxyphenyl)propenone

E-3-[4-Amino-3-ethoxyphenyl)-2-chloro-1-(2,4,5-trimethoxyphenyl)propenone

E-3-[4-Amino-3-ethoxyphenyl)-2-bromo-1-(2,4,5-trimethoxyphenyl)propenone

E-3-[4-Amino-3-ethoxyphenyl)-2-methyl-1-(2,4,5-trimethoxyphenyl)propenone

E-3-[4-Amino-3-ethoxyphenyl)-2-ethyl-1-(2,4,5-trimethoxyphenyl)propenone

E-3-[4-Amino-3-ethoxyphenyl)-2-propyl-1-(2,4,5-trimethoxyphenyl)propenone

E-3-[4-Amino-3-ethoxyphenyl)-2-(1-methylethyl)-1-(2,4,5-trimethoxyphenyl)propenone

E-3-[4-Amino-3-(N,N-dimethylamino)phenyl]-2-chloro-1-(2,4,5-trimethoxyphenyl)propenone

E-3-[4-Amino-3-(N,N-dimethylamino)phenyl]-2-bromo-1-(2,4,5-trimethoxyphenyl)propenone

E-3-[4-Amino-3-(N,N-dimethylamino)phenyl]-2-methyl-1-(2,4,5-trimethoxyphenyl)propenone

E-3-[4-Amino-3-(N,N-dimethylamino)phenyl]-2-ethyl-1-(2,4,5-trimethoxyphenyl)propenone

E-3-[4-Amino-3-(N,N-dimethylamino)phenyl]-2-propyl-1-(2,4,5-trimethoxyphenyl)propenone

E-3-[4-Amino-3-(N,N-dimethylamino)phenyl]-2-(1-methylethyl)-1-(2,4,5-trimethoxyphenyl)propenone

E-3-[3-(N,N-Dimethylamino)-4-methoxyphenyl]-2-chloro-1-(2,4,5-trimethoxyphenyl)propenone

E-3-[3-(N,N-Dimethylamino)-4-methoxyphenyl]-2-bromo-1-(2,4,5-trimethoxyphenyl)propenone

E-3-[3-(N,N-Dimethylamino)-4-methoxyphenyl]-2-methyl-1-(2,4,5-trimethoxyphenyl)propenone

E-3-[3-(N,N-Dimethylamino)-4-methoxyphenyl]-2-ethyl-1-(2,4,5-trimethoxyphenyl)propenone

E-3-[4-Amino-3-(N,N-dimethylamino)-4-methoxyphenyl]-2-propyl-1-(2,3,4-trimethoxyphenyl)propenone

E-3-[4-Amino-3-(N,N-dimethylamino)-4-methoxyphenyl]-2-(1-methylethyl)-1-(2,4,5-trimethoxyphenyl)propenone

E-3-[3-(N,N-Dimethylamino)-4-ethoxyphenyl]-2-chloro-1-(2,4,5-trimethoxyphenyl)propenone

E-3-[3-(N,N-Dimethylamino)-4-ethoxyphenyl]-2-bromo-1-(2,4,5-trimethoxyphenyl)propenone

E-3-[3-(N,N-Dimethylamino)-4-ethoxyphenyl]-2-methyl-1-(2,4,5-trimethoxyphenyl)propenone

E-3-[3-(N,N-Dimethylamino)-4-ethoxyphenyl]-2-ethyl-1-(2,4,5-trimethoxyphenyl)propenone

E-3-[4-Amino-3-(N,N-dimethylamino)-4-ethoxyphenyl]-2-propyl-1-(2,4,5-trimethoxyphenyl)propenone

E-3-[4-Amino-3-(N,N-dimethylamino)-4-ethoxyphenyl]-2-(1-methylethyl)-1-(2,4,5-trimethoxyphenyl)propenone

E-3-[3-Amino-4-methoxyphenyl)-2-chloro-1-(2,3,4,5-tetramethoxyphenyl)propenone

E-3-[3-Amino-4-methoxyphenyl)-2-bromo-1-(2,3,4,5-tetramethoxyphenyl)propenone

E-3-[3-Amino-4-methoxyphenyl)-2-methyl-1-(2,3,4,5-tetramethoxyphenyl)propenone

E-3-[3-Amino-4-methoxyphenyl)-2-ethyl-1-(2,3,4,5-tetramethoxyphenyl)propenone

E-3-[3-Amino-4-methoxyphenyl)-2-propyl-1-(2,3,4,5-tetramethoxyphenyl)propenone

E-3-[3-Amino-4-methoxyphenyl)-2-(1-methylethyl)-1-(2,3,4,5-tetramethoxyphenyl)propenone

E-3-[3-Amino-4-ethoxyphenyl)-2-chloro-1-(2,3,4,5-tetramethoxyphenyl)propenone

E-3-[3-Amino-4-ethoxyphenyl)-2-bromo-1-(2,3,4,5-tetramethoxyphenyl)propenone

E-3-[3-Amino-4-ethoxyphenyl)-2-methyl-1-(2,3,4,5-tetramethoxyphenyl)propenone

E-3-[3-Amino-4-ethoxyphenyl)-2-ethyl-1-(2,3,4,5-tetramethoxyphenyl)propenone

E-3-[3-Amino-4-ethoxyphenyl)-2-propyl-1-(2,3,4,5-tetramethoxyphenyl)propenone

E-3-[3-Amino-4-ethoxyphenyl)-2-(1-methylethyl)-1-(2,3,4,5-tetramethoxyphenyl)propenone

E-3-[3-Amino-4-(N,N-dimethylamino)phenyl]-2-chloro-1-(2,3,4,5-tetramethoxyphenyl)propenone

E-3-[3-Amino-4-(N,N-dimethylamino)phenyl]-2-bromo-1-(2,3,4,5-tetramethoxyphenyl)propenone

E-3-[3-Amino-4-(N,N-dimethylamino)phenyl]-2-methyl-1-(2,3,4,5-tetramethoxyphenyl)propenone

E-3-[3-Amino-4-(N,N-dimethylamino)phenyl]-2-ethyl-1-(2,3,4,5-tetramethoxyphenyl)propenone

E-3-[3-Amino-4-(N,N-dimethylamino)phenyl]-2-propyl-1-(2,3,4,5-tetramethoxyphenyl)propenone

E-3-[3-Amino-4-(N,N-dimethylamino)phenyl]-2-(1-methylethyl)-1-(2,3,4,5-tetramethoxyphenyl)propenone

E-3-(4-Amino-3-methoxyphenyl)-2-chloro-1-(2,3,4,5-tetramethoxyphenyl)propenone

E-3-(4-Amino-3-methoxyphenyl)-2-bromo-1-(2,3,4,5-tetramethoxyphenyl)propenone

E-3-(4-Amino-3-methoxyphenyl)-2-methyl-1-(2,3,4,5-tetramethoxyphenyl)propenone

E-3-(4-Amino-3-methoxyphenyl)-2-ethyl-1-(2,3,4,5-tetramethoxyphenyl)propenone

E-3-(4-Amino-3-methoxyphenyl)-2-propyl-1-(2,3,4,5-tetramethoxyphenyl)propenone

E-3-(4-Amino-3-methoxyphenyl)-2-(1-methylethyl)-1-(2,3,4,5-tetramethoxyphenyl)propenone

E-3-(4-Amino-3-ethoxyphenyl)-2-chloro-1-(2,3,4,5-tetramethoxyphenyl)propenone

E-3-(4-Amino-3-ethoxyphenyl)-2-bromo-1-(2,3,4,5-tetramethoxyphenyl)propenone

E-3-(4-Amino-3-ethoxyphenyl)-2-methyl-1-(2,3,4,5-tetramethoxyphenyl)propenone

E-3-(4-Amino-3-ethoxyphenyl)-2-ethyl-1-(2,3,4,5-tetramethoxyphenyl)propenone

E-3-(4-Amino-3-ethoxyphenyl)-2-propyl-1-(2,3,4,5-tetramethoxyphenyl)propenone

E-3-(4-Amino-3-ethoxyphenyl)-2-(1-methylethyl)-1-(2,3,4,5-tetramethoxyphenyl)propenone

E-3-[4-Amino-3-(N,N-dimethylamino)phenyl]-2-chloro-1-(2,3,4,5-tetramethoxyphenyl)propenone

E-3-[4-Amino-3-(N,N-dimethylamino)phenyl]-2-bromo-1-(2,3,4,5-tetramethoxyphenyl)propenone

E-3-[4-Amino-3-(N,N-dimethylamino)phenyl]-2-methyl-1-(2,3,4,5-tetramethoxyphenyl)propenone

E-3-[4-Amino-3-(N,N-dimethylamino)phenyl]-2-ethyl-1-(2,3,4,5-tetramethoxyphenyl)propenone

E-3-[4-Amino-3-(N,N-dimethylamino)phenyl]-2-propyl-1-(2,3,4,5-tetramethoxyphenyl)propenone

E-3-[4-Amino-3-(N,N-dimethylamino)phenyl]-2-(1-methylethyl)-1-(2,3,4,5-tetramethoxyphenyl)propenone

E-3-[3-(N,N-Dimethylamino)-4-methoxyphenyl]-2-chloro-1-(2,3,4,5-tetramethoxyphenyl)propenone

E-3-[3-(N,N-Dimethylamino)-4-methoxyphenyl]-2-bromo-1-(2,3,4,5-tetramethoxyphenyl)propenone

E-3-[3-(N,N-Dimethylamino)-4-methoxyphenyl]-2-methyl-1-(2,3,4,5-tetramethoxyphenyl)propenone

E-3-[3-(N,N-Dimethylamino)-4-methoxyphenyl]-2-ethyl-1-(2,3,4,5-tetramethoxyphenyl)propenone

E-3-[4-Amino-3-(N,N-dimethylamino)-4-methoxyphenyl]-2-propyl-1-(2,3,4-trimethoxyphenyl)propenone

E-3-[4-Amino-3-(N,N-dimethylamino)-4-methoxyphenyl]-2-(1-methylethyl)-1-(2,3,4,5-tetramethoxyphenyl)propenone

E-3-[3-(N,N-Dimethylamino)-4-ethoxyphenyl]-2-chloro-1-(2,3,4,5-tetramethoxyphenyl)propenone

E-3-[3-(N,N-Dimethylamino)-4-ethoxyphenyl]-2-bromo-1-(2,3,4,5-tetramethoxyphenyl)propenone

E-3-[3-(N,N-Dimethylamino)-4-ethoxyphenyl]-2-methyl-1-(2,3,4,5-tetramethoxyphenyl)propenone

E-3-[3-(N,N-Dimethylamino)-4-ethoxyphenyl]-2-ethyl-1-(2,3,4,5-tetramethoxyphenyl)propenone

E-3-[4-Amino-3-(N,N-dimethylamino)-4-ethoxyphenyl]-2-propyl-1-(2,3,4,5-tetramethoxyphenyl)propenone

E-3-[4-Amino-3-(N,N-dimethylamino)-4-ethoxyphenyl]-2-(1-methylethyl)-1-(2,3,4,5-tetramethoxyphenyl)propenone

E-3-(3-Amino-4-methoxyphenyl)-2-chloro-1-(3-methoxy-4,5-methylenedioxyphenyl)propenone

E-3-(3-Amino-4-methoxyphenyl)-2-bromo-1-(3-methoxy-4,5-methylenedioxyphenyl)propenone

E-3-(3-Amino-4-methoxyphenyl)-2-methyl-1-(3-methoxy-4,5-methylenedioxyphenyl)propenone

E-3-(3-Amino-4-methoxyphenyl)-2-ethyl-1-(3-methoxy-4,5-methylenedioxyphenyl)propenone

E-3-(3-Amino-4-methoxyphenyl)-2-propyl-1-(3-methoxy-4,5-methylenedioxyphenyl)propenone

E-3-(3-Amino-4-methoxyphenyl)-2-(1-methylethyl)-1-(3-methoxy-4,5-methylenedioxyphenyl)propenone

E-3-(3-Amino-4-ethoxyphenyl)-2-chloro-1-(3-methoxy-4,5-methylenedioxyphenyl)propenone

E-3-(3-Amino-4-ethoxyphenyl)-2-bromo-1-(3-methoxy-4,5-methylenedioxyphenyl)propenone

E-3-(3-Amino-4-ethoxyphenyl)-2-methyl-1-(3-methoxy-4,5-methylenedioxyphenyl)propenone

E-3-(3-Amino-4-ethoxyphenyl)-2-ethyl-1-(3-methoxy-4,5-methylenedioxyphenyl)propenone

E-3-(3-Amino-4-ethoxyphenyl)-2-propyl-1-(3-methoxy-4,5-methylenedioxyphenyl)propenone

E-3-(3-Amino-4-ethoxyphenyl)-2-(1-methylethyl)-1-(3-methoxy-4,5-methylenedioxyphenyl)propenone

E-3-[3-Amino-4-(N,N-dimethylamino)phenyl]-2-chloro-1-(3-methoxy-4,5-methylenedioxyphenyl)propenone

E-3-[3-Amino-4-(N,N-dimethylamino)phenyl]-2-bromo-1-(3-methoxy-4,5-methylenedioxyphenyl)propenone

E-3-[3-Amino-4-(N,N-dimethylamino)phenyl]-2-methyl-1-(3-methoxy-4,5-methylenedioxyphenyl)propenone

E-3-[3-Amino-4-(N,N-dimethylamino)phenyl]-2-ethyl-1-(3-methoxy-4,5-methylenedioxyphenyl)propenone

E-3-[3-Amino-4-(N,N-dimethylamino)phenyl]-2-propyl-1-(3-methoxy-4,5-methylenedioxyphenyl)propenone

E-3-[3-Amino-4-(N,N-dimethylamino)phenyl]-2-(1-methylethyl)-1-(3-methoxy-4,5-methylenedioxyphenyl)propenone

E-3-(4-Amino-3-methoxyphenyl)-2-chloro-1-(3-methoxy-4,5-methylenedioxyphenyl)propenone

E-3-(4-Amino-3-methoxyphenyl)-2-bromo-1-(3-methoxy-4,5-methylenedioxyphenyl)propenone

E-3-(4-Amino-3-methoxyphenyl)-2-methyl-1-(3-methoxy-4,5-methylenedioxyphenyl)propenone

E-3-(4-Amino-3-methoxyphenyl)-2-ethyl-1-(3-methoxy-4,5-methylenedioxyphenyl)propenone

E-3-(4-Amino-3-methoxyphenyl)-2-propyl-1-(3-methoxy-4,5-methylenedioxyphenyl)propenone

E-3-(4-Amino-3-methoxyphenyl)-2-(1-methylethyl)-1-(3-methoxy-4,5-methylenedioxyphenyl)propenone

E-3-(4-Amino-3-ethoxyphenyl)-2-chloro-1-(3-methoxy-4,5-methylenedioxyphenyl)propenone

E-3-(4-Amino-3-ethoxyphenyl)-2-bromo-1-(3-methoxy-4,5-methylenedioxyphenyl)propenone

E-3-(4-Amino-3-ethoxyphenyl)-2-methyl-1-(3-methoxy-4,5-methylenedioxyphenyl)propenone

E-3-(4-Amino-3-ethoxyphenyl)-2-ethyl-1-(3-methoxy-4,5-methylenedioxyphenyl)propenone

E-3-(4-Amino-3-ethoxyphenyl)-2-ethyl-1-(3-methoxy-4,5-methylenedioxyphenyl)propenone

E-3-(4-Amino-3-ethoxyphenyl)-2-(1-methylethyl)-1-(3-methoxy-4,5-methylenedioxyphenyl)propenone

E-3-[4-Amino-3-(N,N-dimethylamino)phenyl]-2-chloro-1-(3-methoxy-4,5-methylenedioxyphenyl)propenone

E-3-[4-Amino-3-(N,N-dimethylamino)phenyl]-2-bromo-1-(3-methoxy-4,5-methylenedioxyphenyl)propenone

E-3-[4-Amino-3-(N,N-dimethylamino)phenyl]-2-methyl-1-(3-methoxy-4,5-methylenedioxyphenyl)propenone

E-3-[4-Amino-3-(N,N-dimethylamino)phenyl]-2-ethyl-1-(3-methoxy-4,5-methylenedioxyphenyl)propenone

E-3-[4-Amino-3-(N,N-dimethylamino)phenyl]-2-propyl-1-(3-methoxy-4,5-methylenedioxyphenyl)propenone

E-3-[4-Amino-3-(N,N-dimethylamino)phenyl]-2-(1-methylethyl)-1-(3-methoxy-4,5-methylenedioxyphenyl)propenone

E-3-[3-(N,N-Dimethylamino)-4-methoxyphenyl]-2-chloro-1-(3-methoxy-4,5-methylenedioxyphenyl)propenone

E-3-[3-(N,N-Dimethylamino)-4-methoxyphenyl]-2-bromo-1-(3-methoxy-4,5-methylenedioxyphenyl)propenone

E-3-[3-(N,N-Dimethylamino)-4-methoxyphenyl]-2-methyl-1-(3-methoxy-4,5-methylenedioxyphenyl)propenone

E-3-[3-(N,N-Dimethylamino)-4-methoxyphenyl]-2-ethyl-1-(3-methoxy-4,5-methylenedioxyphenyl)propenone

E-3-[4-Amino-3-(N,N-dimethylamino)-4-methoxyphenyl]-2-(1-methylethyl)-1-(3-methoxy-4,5-methylenedioxyphenyl)propenone

E-3-[3-(N,N-Dimethylamino)-4-ethoxyphenyl]-2-chloro-1-(3-methoxy-4,5-methylenedioxyphenyl)propenone

E-3-[3-(N,N-Dimethylamino)-4-ethoxyphenyl]-2-bromo-1-(3-methoxy-4,5-methylenedioxyphenyl)propenone

E-3-[3-(N,N-Dimethylamino)-4-ethoxyphenyl]-2-methyl-1-(3-methoxy-4,5-methylenedioxyphenyl)propenone

E-3-[3-(N,N-Dimethylamino)-4-ethoxyphenyl]-2-ethyl-1-(3-methoxy-4,5-methylenedioxyphenyl)propenone

E-3-[4-Amino-3-(N,N-dimethylamino)-4-ethoxyphenyl]-2-propyl-1-(3-methoxy-4,5-methylenedioxyphenyl)propenone

E-3-[4-Amino-3-(N,N-dimethylamino)-4-ethoxyphenyl]-2-(1-methylethyl)-1-(3-methoxy-4,5-methylenedioxyphenyl)propenone

E-3-(3-Amino-4-methoxyphenyl)-2-chloro-1-(3-methoxy-4,5-ethylenedioxyphenyl)propenone

E-3-(3-Amino-4-methoxyphenyl)-2-bromo-1-(3-methoxy-4,5-ethylenedioxyphenyl)propenone

E-3-(3-Amino-4-methoxyphenyl)-2-methyl-1-(3-methoxy-4,5-ethylenedioxyphenyl)propenone

E-3-(3-Amino-4-methoxyphenyl)-2-ethyl-1-(3-methoxy-4,5-ethylenedioxyphenyl)propenone

E-3-(3-Amino-4-methoxyphenyl)-2-propyl-1-(3-methoxy-4,5-ethylenedioxyphenyl)propenone

E-3-(3-Amino-4-methoxyphenyl)-2-(1-methylethyl)-1-(3-methoxy-4,5-ethylenedioxyphenyl)propenone

E-3-(3-Amino-4-ethoxyphenyl)-2-chloro-1-(3-methoxy-4,5-ethylenedioxyphenyl)propenone

E-3-(3-Amino-4-ethoxyphenyl)-2-bromo-1-(3-methoxy-4,5-ethylenedioxyphenyl)propenone

E-3-(3-Amino-4-ethoxyphenyl)-2-methyl-1-(3-methoxy-4,5-ethylenedioxyphenyl)propenone

E-3-(3-Amino-4-ethoxyphenyl)-2-ethyl-1-(3-methoxy-4,5-ethylenedioxyphenyl)propenone

E-3-(3-Amino-4-ethoxyphenyl)-2-propyl-1-(3-methoxy-4,5-ethylenedioxyphenyl)propenone

E-3-(3-Amino-4-ethoxyphenyl)-2-(1-methylethyl)-1-(3-methoxy-4,5-ethylenedioxyphenyl)propenone

E-3-[3-Amino-4-(N,N-dimethylamino)phenyl]-2-chloro-1-(3-methoxy-4,5-ethylenedioxyphenyl)propenone

E-3-[3-Amino-4-(N,N-dimethylamino)phenyl]-2-bromo-1-(3-methoxy-4,5-ethylenedioxyphenyl)propenone

E-3-[3-Amino-4-(N,N-dimethylamino)phenyl]-2-methyl-1-(3-methoxy-4,5-ethylenedioxyphenyl)propenone

E-3-[3-Amino-4-(N,N-dimethylamino)phenyl]-2-ethyl-1-(3-methoxy-4,5-ethylenedioxyphenyl)propenone

E-3-[3-Amino-4-(N,N-dimethylamino)phenyl]-2-propyl-1-(3-methoxy-4,5-ethylenedioxyphenyl)propenone

E-3-[3-Amino-4-(N,N-dimethylamino)phenyl]-2-(1-methylethyl)-1-(3-methoxy-4,5-ethylenedioxyphenyl)propenone

E-3-(4-Amino-3-methoxyphenyl)-2-chloro-1-(3-methoxy-4,5-ethylenedioxyphenyl)propenone

E-3-(4-Amino-3-methoxyphenyl)-2-bromo-1-(3-methoxy-4,5-ethylenedioxyphenyl)propenone

E-3-(4-Amino-3-methoxyphenyl)-2-methyl-1-(3-methoxy-4,5-ethylenedioxyphenylphenyl)propenone

E-3-(4-Amino-3-methoxyphenyl)-2-ethyl-1-(3-methoxy-4,5-ethylenedioxyphenyl)propenone

E-3-(4-Amino-3-methoxyphenyl)-2-propyl-1-(3-methoxy-4,5-ethylenedioxyphenyl)propenone

E-3-(4-Amino-3-methoxyphenyl)-2-(1-methylethyl)-1-(3-methoxy-4,5-ethylenedioxyphenyl)propenone

E-3-(4-Amino-3-ethoxyphenyl)-2-chloro-1-(3-methoxy-4,5-ethylenedioxyphenyl)propenone

E-3-(4-Amino-3-ethoxyphenyl)-2-bromo-1-(3-methoxy-4,5-ethylenedioxyphenyl)propenone

E-3-(4-Amino-3-ethoxyphenyl)-2-methyl-1-(3-methoxy-4,5-ethylenedioxyphenyl)propenone

E-3-(4-Amino-3-ethoxyphenyl)-2-ethyl-1-(3-methoxy-4,5-ethylenedioxyphenyl)propenone

E-3-(4-Amino-3-ethoxyphenyl)-2-propyl-1-(3-methoxy-4,5-ethylenedioxyphenyl)propenone

E-3-(4-Amino-3-ethoxyphenyl)-2-(1-methylethyl)-1-(3-methoxy-4,5-ethylenedioxyphenyl)propenone

E-3-[4-Amino-3-(N,N-dimethylamino)phenyl]-2-chloro-1-(3-methoxy-4,5-ethylenedioxyphenyl)propenone

E-3-[4-Amino-3-(N,N-dimethylamino)phenyl]-2-bromo-1-(3-methoxy-4,5-ethylenedioxyphenyl)propenone

E-3-[4-Amino-3-(N,N-dimethylamino)phenyl]-2-methyl-1-(3-methoxy-4,5-ethylenedioxyphenyl)propenone

E-3-[4-Amino-3-(N,N-dimethylamino)phenyl]-2-ethyl-1-(3-methoxy-4,5-ethylenedioxyphenyl)propenone

E-3-[4-Amino-3-(N,N-dimethylamino)phenyl]-2-propyl-1-(3-methoxy-4,5-ethylenedioxyphenyl)propenone

E-3-[4-Amino-3-(N,N-dimethylamino)phenyl]-2-(1-methylethyl)-1-(3-methoxy-4,5-ethylenedioxyphenyl)propenone

E-3-[3-(N,N-Dimethylamino)-4-methoxyphenyl]-2-chloro-1-(3-methoxy-4,5-ethylenedioxyphenyl)propenone

E-3-[3-(N,N-Dimethylamino)-4-methoxyphenyl]-2-bromo-1-(3-methoxy-4,5-ethylenedioxyphenyl)propenone

E-3-[3-(N,N-Dimethylamino)-4-methoxyphenyl]-2-methyl-1-(3-methoxy-4,5-ethylenedioxyphenyl)propenone

E-3-[3-(N,N-Dimethylamino)-4-methoxyphenyl]-2-ethyl-1-(3-methoxy-4,5-ethylenedioxyphenyl)propenone

E-3-[4-Amino-3-(N,N-dimethylamino)-4-methoxyphenyl]-2-(1-methylethyl)-1-(3-methoxy-4,5-ethylenedioxyphenyl)propenone

E-3-[3-(N,N-Dimethylamino)-4-ethoxyphenyl]-2-chloro-1-(3-methoxy-4,5-ethylenedioxyphenyl)propenone

E-3-[3-(N,N-Dimethylamino)-4-ethoxyphenyl]-2-bromo-1-(3-methoxy-4,5-ethylenedioxyphenyl)propenone

E-3-[3-(N,N-Dimethylamino)-4-ethoxyphenyl]-2-methyl-1-(3-methoxy-4,5-ethylenedioxyphenyl)propenone

E-3-[3-(N,N-Dimethylamino)-4-ethoxyphenyl]-2-ethyl-1-(3-methoxy-4,5-ethylenedioxyphenyl)propenone

E-3-[4-Amino-3-(N,N-dimethylamino)-4-ethoxyphenyl]-2-propyl-1-(3-methoxy-4,5-ethylenedioxyphenyl)propenone

E-3-[4-Amino-3-(N,N-dimethylamino)-4-ethoxyphenyl]-2-(1-methylethyl)-1-(3-methoxy-4,5-ethylenedioxyphenyl)propenone

E-3-(3-Amino-4-methoxyphenyl)-2-chloro-1-(2-methoxy-3,4-methylenedioxyphenyl)propenone

E-3-(3-Amino-4-methoxyphenyl)-2-bromo-1-(2-methoxy-3,4-methylenedioxyphenyl)propenone

E-3-(3-Amino-4-methoxyphenyl)-2-methyl-1-(2-methoxy-3,4-methylenedioxyphenyl)propenone

E-3-(3-Amino-4-methoxyphenyl)-2-ethyl-1-(2-methoxy-3,4-methylenedioxyphenyl)propenone

E-3-(3-Amino-4-methoxyphenyl)-2-propyl-1-(2-methoxy-3,4-methylenedioxyphenyl)propenone

E-3-(3-Amino-4-methoxyphenyl)-2-(1-methylethyl)-1-(2-methoxy-3,4-methylenedioxyphenyl)propenone

E-3-(3-Amino-4-ethoxyphenyl)-2-chloro-1-(2-methoxy-3,4-methylenedioxyphenyl)propenone

E-3-(3-Amino-4-ethoxyphenyl)-2-bromo-1-(2-methoxy-3,4-methylenedioxyphenyl)propenone

E-3-(3-Amino-4-ethoxyphenyl)-2-methyl-1-(2-methoxy-3,4-methylenedioxyphenyl)propenone

E-3-(3-Amino-4-ethoxyphenyl)-2-ethyl-1-(2-methoxy-3,4-methylenedioxyphenyl)propenone

E-3-(3-Amino-4-ethoxyphenyl)-2-propyl-1-(2-methoxy-3,4-methylenedioxyphenyl)propenone

E-3-(3-Amino-4-ethoxyphenyl)-2-(1-methylethyl)-1-(2-methoxy-3,4-methylenedioxyphenyl)propenone

E-3-[3-Amino-4-(N,N-dimethylamino)phenyl]-2-chloro-1-(2-methoxy-3,4-methylenedioxyphenyl)propenone

E-3-[3-Amino-4-(N,N-dimethylamino)phenyl]-2-bromo-1-(2-methoxy-3,4-methylenedioxyphenyl)propenone

E-3-[3-Amino-4-(N,N-dimethylamino)phenyl]-2-methyl-1-(2-methoxy-3,4-methylenedioxyphenyl)propenone

E-3-[3-Amino-4-(N,N-dimethylamino)phenyl]-2-ethyl-1-(2-methoxy-3,4-methylenedioxyphenyl)propenone

E-3-[3-Amino-4-(N,N-dimethylamino)phenyl]-2-propyl-1-(2-methoxy-3,4-methylenedioxyphenyl)propenone

E-3-[3-Amino-4-(N,N-dimethylamino)phenyl]-2-(1-methylethyl)-1-(2-methoxy-3,4-methylenedioxyphenyl)propenone

E-3-(4-Amino-3-methoxyphenyl]-2-chloro-1-(2-methoxy-3,4-methylenedioxyphenyl)propenone

E-3-(4-Amino-3-methoxyphenyl]-2-bromo-1-(2-methoxy-3,4-methylenedioxyphenyl)propenone

E-3-(4-Amino-3-methoxyphenyl]-2-methyl-1-(2-methoxy-3,4-methylenedioxyphenyl)propenone

E-3-(4-Amino-3-methoxyphenyl]-2-ethyl-1-(2-methoxy-3,4-methylenedioxyphenyl)propenone

E-3-(4-Amino-3-methoxyphenyl]-2-propyl-1-(2-methoxy-3,4-methylenedioxyphenyl)propenone

E-3-(4-Amino-3-methoxyphenyl]-2-(1-methylethyl)-1-(2-methoxy-3,4-methylenedioxyphenyl)propenone

E-3-(4-Amino-3-ethoxyphenyl]-2-chloro-1-(2-methoxy-3,4-methylenedioxyphenyl)propenone

E-3-(4-Amino-3-ethoxyphenyl]-2-bromo-1-(2-methoxy-3,4-methylenedioxyphenyl)propenone

E-3-(4-Amino-3-ethoxyphenyl]-2-methyl-1-(2-methoxy-3,4-methylenedioxyphenyl)propenone

E-3-(4-Amino-3-ethoxyphenyl]-2-ethyl-1-(2-methoxy-3,4-methylenedioxyphenyl)propenone

E-3-(4-Amino-3-ethoxyphenyl]-2-propyl-1-(2-methoxy-3,4-methylenedioxyphenyl)propenone

E-3-(4-Amino-3-ethoxyphenyl]-2-(1-methylethyl)-1-(2-methoxy-3,4-methylenedioxyphenyl)propenone

E-3-[4-Amino-3-(N,N-dimethylamino)phenyl]-2-chloro-1-(2-methoxy-3,4-methylenedioxyphenyl)propenone

E-3-[4-Amino-3-(N,N-dimethylamino)phenyl]-2-bromo-1-(2-methoxy-3,4-methylenedioxyphenyl)propenone

E-3-[4-Amino-3-(N,N-dimethylamino)phenyl]-2-methyl-1-(2-methoxy-3,4-methylenedioxyphenyl)propenone

E-3-[4-Amino-3-(N,N-dimethylamino)phenyl]-2-ethyl-1-(2-methoxy-3,4-methylenedioxyphenyl)propenone

E-3-[4-Amino-3-(N,N-dimethylamino)phenyl]-2-propyl-1-(2-methoxy-3,4-methylenedioxyphenyl)propenone

E-3-[4-Amino-3-(N,N-dimethylamino)phenyl]-2-(1-methylethyl)-1-(2-methoxy-3,4-methylenedioxyphenyl)propenone

E-3-[3-(N,N-Dimethylamino)-4-methoxyphenyl]-2-chloro-1-(2-methoxy-3,4-methylenedioxyphenyl)propenone

E-3-[3-(N,N-Dimethylamino)-4-methoxyphenyl]-2-bromo-1-(2-methoxy-3,4-methylenedioxyphenyl)propenone

E-3-[3-(N,N-Dimethylamino)-4-methoxyphenyl]-2-methyl-1-(2-methoxy-3,4-methylenedioxyphenyl)propenone

E-3-[3-(N,N-Dimethylamino)-4-methoxyphenyl]-2-ethyl-1-(2-methoxy-3,4-methylenedioxyphenyl)propenone

E-3-[4-Amino-3-(N,N-dimethylamino)-4-methoxyphenyl]-2-(1-methylethyl)-1-(2-methoxy-3,4-methylenedioxyphenyl)propenone

E-3-[3-(N,N-Dimethylamino)-4-ethoxyphenyl]-2-chloro-1-(2-methoxy-3,4-methylenedioxyphenyl)propenone

E-3-[3-(N,N-Dimethylamino)-4-ethoxyphenyl]-2-bromo-1-(2-methoxy-3,4-methylenedioxyphenyl)propenone

E-3-[3-(N,N-Dimethylamino)-4-ethoxyphenyl]-2-methyl-1-(2-methoxy-3,4-methylenedioxyphenyl)propenone

E-3-[3-(N,N-Dimethylamino)-4-ethoxyphenyl]-2-ethyl-1-(2-methoxy-3,4-methylenedioxyphenyl)propenone

E-3-[4-Amino-3-(N,N-Dimethylamino)-4-ethoxyphenyl]-2-propyl-1-(2-methoxy-3,4-methylenedioxyphenyl)propenone

E-3-[4-Amino-3-(N,N-Dimethylamino)-4-ethoxyphenyl]-2-(1-methylethyl)-1-(2-methoxy-3,4-methylenedioxyphenyl)propenone

E-3-(3-Amino-4-methoxyphenyl)-2-chloro-1-(2-methoxy-3,4-ethylenedioxyphenyl)propenone

E-3-(3-Amino-4-methoxyphenyl)-2-bromo-1-(2-methoxy-3,4-ethylenedioxyphenyl)propenone

E-3-(3-Amino-4-methoxyphenyl)-2-methyl-1-(2-methoxy-3,4-ethylenedioxyphenyl)propenone

E-3-(3-Amino-4-methoxyphenyl)—2-ethyl-1-(2-methoxy-3,4-ethylenedioxyphenyl)propenone

E-3-(3-Amino-4-methoxyphenyl)-2-propyl-1-(2-methoxy-3,4-ethylenedioxyphenyl)propenone

E-3-(3-Amino-4-methoxyphenyl)-2-(1-methylethyl)-1-(2-methoxy-3,4-ethylenedioxyphenyl)propenone

E-3-(3-Amino-4-ethoxyphenyl)-2-chloro-1-(2-methoxy-3,4-ethylenedioxyphenyl)propenone

E-3-(3-Amino-4-ethoxyphenyl)-2-bromo-1-(2-methoxy-3,4-ethylenedioxyphenyl)propenone

E-3-(3-Amino-4-ethoxyphenyl)-2-methyl-1-(2-methoxy-3,4-ethylenedioxyphenyl)propenone

E-3-(3-Amino-4-ethoxyphenyl)—2-ethyl-1-(2-methoxy-3,4-ethylenedioxyphenyl)propenone

E-3-(3-Amino-4-ethoxyphenyl)-2-propyl-1-(2-methoxy-3,4-ethylenedioxyphenyl)propenone

E-3-(3-Amino-4-ethoxyphenyl)-2-(1-methylethyl)-1-(2-methoxy-3,4-ethylenedioxyphenyl)propenone

E-3-[3-Amino-4-(N,N-dimethylamino)phenyl]-2-chloro-1-(2-methoxy-3,4-ethylenedioxyphenyl)propenone

E-3-[3-Amino-4-(N,N-dimethylamino)phenyl]-2-bromo-1-(2-methoxy-3,4-ethylenedioxyphenyl)propenone

E-3-[3-Amino-4-(N,N-dimethylamino)phenyl]-2-methyl-1-(2-methoxy-3,4-ethylenedioxyphenyl)propenone

E-3-[3-Amino-4-(N,N-dimethylamino)phenyl]-2-ethyl-1-(2-methoxy-3,4-ethylenedioxyphenyl)propenone

E-3-[3-Amino-4-(N,N-dimethylamino)phenyl]-2-propyl-1-(2-methoxy-3,4-ethylenedioxyphenyl)propenone

E-3-[3-Amino-4-(N,N-dimethylamino)phenyl]-2-(1-methylethyl)-1-(2-methoxy-3,4-ethylenedioxyphenyl)propenone

E-3-(4-Amino-3-methoxyphenyl)-2-chloro-1-(2-methoxy-3,4-ethylenedioxyphenyl)propenone

E-3-(4-Amino-3-methoxyphenyl)-2-bromo-1-(2-methoxy-3,4-ethylenedioxyphenyl)propenone

E-3-(4-Amino-3-methoxyphenyl)-2-methyl-1-(2-methoxy-3,4-ethylenedioxyphenyl)propenone

E-3-(4-Amino-3-methoxyphenyl)-2-ethyl-1-(2-methoxy-3,4-ethylenedioxyphenyl)propenone

E-3-(4-Amino-3-methoxyphenyl)-2-propyl-1-(2-methoxy-3,4-ethylenedioxyphenyl)propenone

E-3-(4-Amino-3-methoxyphenyl)-2-(1-methylethyl)-1-(2-methoxy-3,4-ethylenedioxyphenyl)propenone

E-3-(4-Amino-3-ethoxyphenyl)-2-chloro-1-(2-methoxy-3,4-ethylenedioxyphenyl)propenone

E-3-(4-Amino-3-ethoxyphenyl)-2-bromo-1-(2-methoxy-3,4-ethylenedioxyphenyl)propenone

E-3-(4-Amino-3-ethoxyphenyl)-2-methyl-1-(2-methoxy-3,4-ethylenedioxyphenyl)propenone

E-3-(4-Amino-3-ethoxyphenyl)-2-ethyl-1-(2-methoxy-3,4-ethylenedioxyphenyl)propenone

E-3-(4-Amino-3-ethoxyphenyl)-2-propyl-1-(2-methoxy-3,4-ethylenedioxyphenyl)propenone

E-3-(4-Amino-3-ethoxyphenyl)-2-(1-methylethyl)-1-(2-methoxy-3,4-ethylenedioxyphenyl)propenone

E-3-[4-Amino-3-(N,N-dimethylamino)phenyl]-2-chloro-1-(2-methoxy-3,4-ethylenedioxyphenyl)propenone

E-3-[4-Amino-3-(N,N-dimethylamino)phenyl]-2-bromo-1-(2-methoxy-3,4-ethylenedioxyphenyl)propenone

E-3-[4-Amino-3-(N,N-dimethylamino)phenyl]-2-methyl-1-(2-methoxy-3,4-ethylenedioxyphenyl)propenone

E-3-[4-Amino-3-(N,N-dimethylamino)phenyl]-2-ethyl-1-(2-methoxy-3,4-ethylenedioxyphenyl)propenone

E-3-[4-Amino-3-(N,N-dimethylamino)phenyl]-2-propyl-1-(2-methoxy-3,4-ethylenedioxyphenyl)propenone

E-3-[4-Amino-3-(N,N-dimethylamino)phenyl]-2-(1-methylethyl)-1-(2-methoxy-3,4-ethylenedioxyphenyl)propenone

E-3-[3-(N,N-Dimethylamino)-4-methoxyphenyl]-2-chloro-1-(2-methoxy-3,4-ethylenedioxyphenyl)propenone

E-3-[3-(N,N-Dimethylamino)-4-methoxyphenyl]-2-bromo-1-(2-methoxy-3,4-ethylenedioxyphenyl)propenone

E-3-[3-(N,N-Dimethylamino)-4-methoxyphenyl]-2-methyl-1-(2-methoxy-3,4-ethylenedioxyphenyl)propenone

E-3-[3-(N,N-Dimethylamino)-4-methoxyphenyl]-2-ethyl-1-(2-methoxy-3,4-ethylenedioxyphenyl)propenone

E-3-[4-Amino-3-(N,N-dimethylamino)-4-methoxyphenyl]-2-(1-methylethyl)-1-(2-methoxy-3,4-ethylenedioxyphenyl)propenone

E-3-[3-(N,N-dimethylamino)-4-ethoxyphenyl]-2-chloro-1-(2-methoxy-3,4-ethylenedioxyphenyl)propenone

E-3-[3-(N,N-dimethylamino)-4-ethoxyphenyl]-2-bromo-1-(2-methoxy-3,4-ethylenedioxyphenyl)propenone

E-3-[3-(N,N-dimethylamino)-4-ethoxyphenyl]-2-methyl-1-(2-methoxy-3,4-ethylenedioxyphenyl)propenone

E-3-[3-(N,N-dimethylamino)-4-ethoxyphenyl]-2-ethyl-1-(2-methoxy-3,4-ethylenedioxyphenyl)propenone

E-3-[4-Amino-3-(N,N-dimethylamino)-4-ethoxyphenyl]-2-propyl-1-(2-methoxy-3,4-ethylenedioxyphenyl)propenone

E-3-[4-Amino-3-(N,N-dimethylamino)-4-ethoxyphenyl]-2-(1-methylethyl)-1-(2-methoxy-3,4-ethylenedioxyphenyl)propenone

E-3-(3-Amino-4-methoxyphenyl]-2-chloro-1-(2-methoxy-4,5-methylenedioxyphenyl)propenone

E-3-(3-Amino-4-methoxyphenyl]-2-bromo-1-(2-methoxy-4,5-methylenedioxyphenyl)propenone

E-3-(3-Amino-4-methoxyphenyl]-2-methyl-1-(2-methoxy-4,5-methylenedioxyphenyl)propenone

E-3-(3-Amino-4-methoxyphenyl]-2-ethyl-1-(2-methoxy-4,5-methylenedioxyphenyl)propenone

E-3-(3-Amino-4-methoxyphenyl]-2-propyl-1-(2-methoxy-4,5-methylenedioxyphenyl)propenone

E-3-(3-Amino-4-methoxyphenyl]-2-(1-methylethyl)-1-(2-methoxy-4,5-methylenedioxyphenyl)propenone

E-3-(3-Amino-4-ethoxyphenyl]-2-chloro-1-(2-methoxy-4,5-methylenedioxyphenyl)propenone

E-3-(3-Amino-4-ethoxyphenyl]-2-bromo-1-(2-methoxy-4,5-methylenedioxyphenyl)propenone

E-3-(3-Amino-4-ethoxyphenyl]-2-methyl-1-(2-methoxy-4,5-methylenedioxyphenyl)propenone

E-3-(3-Amino-4-ethoxyphenyl]-2-ethyl-1-(2-methoxy-4,5-methylenedioxyphenyl)propenone

E-3-(3-Amino-4-ethoxyphenyl]-2-propyl-1-(2-methoxy-4,5-methylenedioxyphenyl)propenone

E-3-(3-Amino-4-ethoxyphenyl]-2-(1-methylethyl)-1-(2-methoxy-4,5-methylenedioxyphenyl)propenone

E-3-[3-Amino-4-(N,N-dimethylamino)phenyl]-2-chloro-1-(2-methoxy-4,5-methylenedioxyphenyl)propenone

E-3-[3-Amino-4-(N,N-dimethylamino)phenyl]-2-bromo-1-(2-methoxy-4,5-methylenedioxyphenyl)propenone

E-3-[3-Amino-4-(N,N-dimethylamino)phenyl]-2-methyl-1-(2-methoxy-4,5-methylenedioxyphenyl)propenone

E-3-[3-Amino-4-(N,N-dimethylamino)phenyl]-2-ethyl-1-(2-methoxy-4,5-methylenedioxyphenyl)propenone

E-3-[3-Amino-4-(N,N-dimethylamino)phenyl]-2-propyl-1-(2-methoxy-4,5-methylenedioxyphenyl)propenone

E-3-[3-Amino-4-(N,N-dimethylamino)phenyl]-2-(1-methylethyl)-1-(2-methoxy-4,5-methylenedioxyphenyl)propenone

E-3-(4-Amino-3-methoxyphenyl)-2-chloro-1-(2-methoxy-4,5-methylenedioxyphenyl)propenone

E-3-(4-Amino-3-methoxyphenyl)-2-bromo-1-(2-methoxy-4,5-methylenedioxyphenyl)propenone

E-3-(4-Amino-3-methoxyphenyl)-2-methyl-1-(2-methoxy-4,5-methylenedioxyphenyl)propenone

E-3-(4-Amino-3-methoxyphenyl)-2-ethyl-1-(2-methoxy-4,5-methylenedioxyphenyl)propenone

E-3-(4-Amino-3-methoxyphenyl)-2-propyl-1-(2-methoxy-4,5-methylenedioxyphenyl)propenone

E-3-(4-Amino-3-methoxyphenyl)-2-(1-methylethyl)-1-(2-methoxy-4,5-methylenedioxyphenyl)propenone

E-3-(4-Amino-3-ethoxyphenyl)-2-chloro-1-(2-methoxy-4,5-methylenedioxyphenyl)propenone

E-3-(4-Amino-3-ethoxyphenyl)-2-bromo-1-(2-methoxy-4,5-methylenedioxyphenyl)propenone

E-3-(4-Amino-3-ethoxyphenyl)-2-methyl-1-(2-methoxy-4,5-methylenedioxyphenyl)propenone

E-3-(4-Amino-3-ethoxyphenyl)-2-ethyl-1-(2-methoxy-4,5-methylenedioxyphenyl)propenone

E-3-(4-Amino-3-ethoxyphenyl)-2-propyl-1-(2-methoxy-4,5-methylenedioxyphenyl)propenone

E-3-(4-Amino-3-ethoxyphenyl)-2-(1-methylethyl)-1-(2-methoxy-4,5-methylenedioxyphenyl)propenone

E-3-[4-Amino-3-(N,N-dimethylamino)phenyl]-2-chloro-1-(2-methoxy-4,5-methylenedioxyphenyl)propenone

E-3-[4-Amino-3-(N,N-dimethylamino)phenyl]-2-bromo-1-(2-methoxy-4,5-methylenedioxyphenyl)propenone

E-3-[4-Amino-3-(N,N-dimethylamino)phenyl]-2-methyl-1-(2-methoxy-4,5-methylenedioxyphenyl)propenone

E-3-[4-Amino-3-(N,N-dimethylamino)phenyl]-2-ethyl-1-(2-methoxy-4,5-methylenedioxyphenyl)propenone

E-3-[4-Amino-3-(N,N-dimethylamino)phenyl]-2-propyl-1-(2-methoxy-4,5-methylenedioxyphenyl)propenone

E-3-[4-Amino-3-(N,N-dimethylamino)phenyl]-2-(1-methylethyl)-1-(2-methoxy-4,5-methylenedioxyphenyl)propenone

E-3-[3-(N,N-Dimethylamino)-4-methoxyphenyl]-2-chloro-1-(2-methoxy-4,5-methylenedioxyphenyl)propenone

E-3-[3-(N,N-Dimethylamino)-4-methoxyphenyl]-2-bromo-1-(2-methoxy-4,5-methylenedioxyphenyl)propenone

E-3-[3-(N,N-Dimethylamino)-4-methoxyphenyl]-2-methyl-1-(2-methoxy-4,5-methylenedioxyphenyl) propenone

E-3-[3-(N,N-Dimethylamino)-4-methoxyphenyl]-2-ethyl-1-(2-methoxy-4,5-methylenedioxyphenyl)propenone

E-3-[4-Amino-3-(N,N-dimethylamino)-4-methoxyphenyl]-2-(1-methylethyl)-1-(2-methoxy-4,5-methylenedioxyphenyl)propenone

E-3-[3-(N,N-dimethylamino)-4-ethoxyphenyl]-2-chloro-1-(2-methoxy-4,5-methylenedioxyphenyl)propenone

E-3-[3-(N,N-dimethylamino)-4-ethoxyphenyl]-2-bromo-1-(2-methoxy-4,5-methylenedioxyphenyl)propenone

E-3-[3-(N,N-dimethylamino)-4-ethoxyphenyl]-2-methyl-1-(2-methoxy-4,5-methylenedioxyphenyl)propenone

E-3-[3-(N,N-dimethylamino)-4-ethoxyphenyl]-2-ethyl-1-(2-methoxy-4,5-methylenedioxyphenyl)propenone

E-3-[4-Amino-3-(N,N-dimethylamino)-4-ethoxyphenyl]-2-propyl-1-(2-methoxy-4,5-methylenedioxyphenyl)propenone

E-3-[4-Amino-3-(N,N-dimethylamino)-4-ethoxyphenyl]-2-(1-methylethyl)-1-(2-methoxy-4,5-methylenedioxyphenyl)propenone

E-3-(3-Amino-4-methoxyphenyl]-2-chloro-1-(2-methoxy-4,5-ethylenedioxyphenyl)propenone

E-3-(3-Amino-4-methoxyphenyl]-2-bromo-1-(2-methoxy-4,5-ethylenedioxyphenyl)propenone

E-3-(3-Amino-4-methoxyphenyl]-2-methyl-1-(2-methoxy-4,5-ethylenedioxyphenyl)propenone

E-3-(3-Amino-4-methoxyphenyl]-2-ethyl-1-(2-methoxy-4,5-ethylenedioxyphenyl)propenone

E-3-(3-Amino-4-methoxyphenyl]-2-propyl-1-(2-methoxy-4,5-ethylenedioxyphenyl)propenone

E-3-(3-Amino-4-methoxyphenyl]-2-(1-methylethyl)-1-(2-methoxy-4,5-ethylenedioxyphenyl)propenone

E-3-(3-Amino-4-ethoxyphenyl]-2-chloro-1-(2-methoxy-4,5-ethylenedioxyphenyl)propenone

E-3-(3-Amino-4-ethoxyphenyl]-2-bromo-1-(2-methoxy-4,5-ethylenedioxyphenyl)propenone

E-3-(3-Amino-4-ethoxyphenyl]-2-methyl-1-(2-methoxy-4,5-ethylenedioxyphenyl)propenone

E-3-(3-Amino-4-ethoxyphenyl]-2-ethyl-1-(2-methoxy-4,5-ethylenedioxyphenyl)propenone

E-3-(3-Amino-4-ethoxyphenyl]-2-propyl-1-(2-methoxy-4,5-ethylenedioxyphenyl)propenone

E-3-(3-Amino-4-ethoxyphenyl]-2-(1-methylethyl)-1-(2-methoxy-4,5-ethylenedioxyphenyl)propenone

E-3-[3-Amino-4-(N,N-dimethylamino)phenyl]-2-chloro-1-(2-methoxy-4,5-ethylenedioxyphenyl)propenone

E-3-[3-Amino-4-(N,N-dimethylamino)phenyl]-2-bromo-1-(2-methoxy-4,5-ethylenedioxyphenyl)propenone

E-3-[3-Amino-4-(N,N-dimethylamino)phenyl]-2-methyl-1-(2-methoxy-4,5-ethylenedioxyphenyl)propenone

E-3-[3-Amino-4-(N,N-dimethylamino)phenyl]-2-ethyl-1-(2-methoxy-4,5-ethylenedioxyphenyl)propenone

E-3-[3-Amino-4-(N,N-dimethylamino)phenyl]-2-propyl-1-(2-methoxy-4,5-ethylenedioxyphenyl)propenone

E-3-[3-Amino-4-(N,N-dimethylamino)phenyl]-2-(1-methylethyl)-1-(2-methoxy-4,5-ethylenedioxyphenyl)propenone

E-3-(4-Amino-3-methoxyphenyl)-2-chloro-1-(2-methoxy-4,5-ethylenedioxyphenyl)propenone

E-3-(4-Amino-3-methoxyphenyl)-2-bromo-1-(2-methoxy-4,5-ethylenedioxyphenyl)propenone

E-3-(4-Amino-3-methoxyphenyl)-2-methyl-1-(2-methoxy-4,5-ethylenedioxyphenyl)propenone

E-3-(4-Amino-3-methoxyphenyl)-2-ethyl-1-(2-methoxy-4,5-ethylenedioxyphenyl)propenone

E-3-(4-Amino-3-methoxyphenyl)-2-propyl-1-(2-methoxy-4,5-ethylenedioxyphenyl)propenone

E-3-(4-Amino-3-methoxyphenyl)-2-(1-methylethyl)-1-(2-methoxy-4,5-ethylenedioxyphenyl)propenone

E-3-(4-Amino-3-ethoxyphenyl)-2-chloro-1-(2-methoxy-4,5-ethylenedioxyphenyl)propenone

E-3-(4-Amino-3-ethoxyphenyl)-2-bromo-1-(2-methoxy-4,5-ethylenedioxyphenyl)propenone

E-3-(4-Amino-3-ethoxyphenyl)-2-methyl-1-(2-methoxy-4,5-ethylenedioxyphenyl)propenone

E-3-(4-Amino-3-ethoxyphenyl)-2-ethyl-1-(2-methoxy-4,5-ethylenedioxyphenyl)propenone

E-3-(4-Amino-3-ethoxyphenyl)-2-propyl-1-(2-methoxy-4,5-ethylenedioxyphenyl)propenone

E-3-(4-Amino-3-ethoxyphenyl)-2-(1-methylethyl)-1-(2-methoxy-4,5-ethylenedioxyphenyl)propenone

E-3-[4-Amino-3-(N,N-dimethylamino)phenyl]-2-chloro-1-(2-methoxy-4,5-ethylenedioxyphenyl)propenone

E-3-[4-Amino-3-(N,N-dimethylamino)phenyl]-2-bromo-1-(2-methoxy-4,5-ethylenedioxyphenyl)propenone

E-3-[4-Amino-3-(N,N-dimethylamino)phenyl]-2-methyl-1-(2-methoxy-4,5-ethylenedioxyphenyl)propenone

E-3-[4-Amino-3-(N,N-dimethylamino)phenyl]-2-ethyl-1-(2-methoxy-4,5-ethylenedioxyphenyl)propenone

E-3-[4-Amino-3-(N,N-dimethylamino)phenyl]-2-propyl-1-(2-methoxy-4,5-ethylenedioxyphenyl)propenone

E-3-[4-Amino-3-(N,N-dimethylamino)phenyl]-2-(1-methylethyl)-1-(2-methoxy-4,5-ethylenedioxyphenyl)propenone

E-3-[3-(N,N-Dimethylamino)-4-methoxyphenyl]-2-chloro-1-(2-methoxy-4,5-ethylenedioxyphenyl)propenone

E-3-[3-(N,N-Dimethylamino)-4-methoxyphenyl]-2-bromo-1-(2-methoxy-4,5-ethylenedioxyphenyl)propenone

E-3-[3-(N,N-Dimethylamino)-4-methoxyphenyl]-2-methyl-1-(2-methoxy-4,5-ethylenedioxyphenyl)propenone

E-3-[3-(N,N-Dimethylamino)-4-methoxyphenyl]-2-ethyl-1-(2-methoxy-4,5-ethylenedioxyphenyl)propenone

E-3-[4-Amino-3-(N,N-Dimethylamino)-4-methoxyphenyl]-2-(1-methylethyl)-1-(2-methoxy-4,5-ethylenedioxyphenyl)propenone

E-3-[3-(N,N-Dimethylamino)-4-ethoxyphenyl]-2-chloro-1-(2-methoxy-4,5-ethylenedioxyphenyl)propenone

E-3-[3-(N,N-Dimethylamino)-4-ethoxyphenyl]-2-bromo-1-(2-methoxy-4,5-ethylenedioxyphenyl)propenone

E-3-[3-(N,N-Dimethylamino)-4-ethoxyphenyl]-2-methyl-1-(2-methoxy-4,5-ethylenedioxyphenyl)propenone

E-3-[3-(N,N-Dimethylamino)-4-ethoxyphenyl]-2-ethyl-1-(2-methoxy-4,5-ethylenedioxyphenyl)propenone

E-3-[4-Amino-3-(N,N-dimethylamino)-4-ethoxyphenyl]-2-propyl-1-(2-methoxy-4,5-ethylenedioxyphenyl)propenone

E-3-[4-Amino-3-(N,N-dimethylamino)-4-ethoxyphenyl]-2-(1-methylethyl)-1-(2-methoxy-4,5-ethylenedioxyphenyl)propenone

2-Aminoacetic acid {2-methoxy-5-[2-methyl-3-oxo-3-(3,4,5-trimethoxyphenyl)propenyl]phenyl}amide

2-Aminopropanoic acid {2-methoxy-5-[2-methyl-3-oxo-3-(3,4,5-trimethoxyphenyl)propenyl]phenyl}amide

2-Amino-5-guanidinopentanoic acid {2-methoxy-5-[2-methyl-3-oxo-3-(3,4,5-trimethoxyphenyl)propenyl]-phenyl}amide

2-Aminosuccinamic acid {2-methoxy-5-[2-methyl-3-oxo-3-(3,4,5-trimethoxyphenyl)propenyl]phenyl}amide

2-Aminosuccinic acid {2-methoxy-5-[2-methyl-3-oxo-3-(3,4,5-trimethoxyphenyl)propenyl]phenyl}amide

2-Amino-3-mercaptopropanoic acid {2-methoxy-5-[2-methyl-3-oxo-3-(3,4,5-trimethoxyphenyl)propenyl]phenyl}amide

2-Aminoglutamic acid {2-methoxy-5-[2-methyl-3-oxo-3-(3,4,5-trimethoxyphenyl)propenyl]phenyl}amide

2-Amino-3-methylpentanoic acid {2-methoxy-5-[2-methyl-3-oxo-3-(3,4,5-trimethoxyphenyl)propenyl]phenyl}amide

2,6-Diaminocaproic acid {2-methoxy-5-[2-methyl-3-oxo-3-(3,4,5-trimethoxyphenyl)propenyl]phenyl}amide

2-Amino-4-methylthiobutanoic acid {2-methoxy-5-[2-methyl-3-oxo-3-(3,4,5-trimethoxyphényl)propenyl]phenyl}amide

2-Aminocaproic acid {2-methoxy-5-[2-methyl-3-oxo-3-(3,4,5-trimethoxyphenyl)propenyl]phenyl}amide

2-Amino-3-phenylpropanoic acid {2-methoxy-5-[2-methyl-3-oxo-3-(3,4,5-trimethoxyphenyl)propenyl]phenyl}amide

Pyrrolidine-2-carboxylic acid {2-methoxy-5-[2-methyl-3-oxo-3-(3,4,5-trimethoxyphenyl)propenyl]phenyl}amide

2-Amino-3-hydroxypropanoic acid {2-methoxy-5-[2-methyl-3-oxo-3-(3,4,5-trimethoxyphenyl)propenyl]phenyl}amide

2-Amino-3-hydroxybutanoic acid {2-methoxy-5-[2-methyl-3-oxo-3-(3,4,5-trimethoxyphenyl)propenyl]phenyl}amide

2-Amino-3-(1-H-indol-5-yl)propanoic acid {2-methoxy-5-[2-methyl-3-oxo-3-(3,4,5-trimethoxyphenyl)propenyl]-phenyl}amide

2-Amino-3-(4-hydroxyphenyl)propanoic acid {2-methoxy-5-[2-methyl-3-oxo-3-(3,4,5-trimethoxyphenyl)propenyl]phenyl}amide

2-Amino-2-methylbutanoic acid {2-methoxy-5-[2-methyl-3-oxo-3-(3,4,5-trimethoxyphenyl)propenyl]phenyl}amide

2-(N-methylamino)acetic acid {2-methoxy-5-[2-methyl-3-oxo-3-(3,4,5-trimethoxyphenyl)propenyl]phenyl}amide

2-Amino-5-[3-(1-nitroguanidino)]pentanoic acid {2-methoxy-5-[2-methyl-3-oxo-3-(3,4,5-trimethoxyphenyl)-propenyl]phenyl}amide

1-Methylpyrrolidine-2-carboxylic acid {2-methoxy-5-[2-methyl-3-oxo-3-(3,4,5-trimethoxyphenyl)propenyl]-phenyl}amide

E-2-Methyl-3-(1-H-indol-5-yl)-1-(3,4,5-trimethoxyphenyl)propenone

E-2-Methyl-3-(1-methyl-1-H-indol-5-yl)-1-(3,4,5-trimethoxyphenyl)propenone;

E-2-Methyl-3-(2,3-dihydro-1-H-indol-5-yl)-1-(3,4,5-trimethoxyphenyl)propenone

E-2-Methyl-3-(1-methyl-2,3-dihydro-1-H-indol-5-yl)-1-(3,4,5-trimethoxyphenyl)propenone

E-2-Methyl-3-(1-hydroxymethyl-1-H-indol-5-yl)-1-(3,4,5-trimethoxyphenyl)propenone

E-2-Methyl-3-(1-hydroxyethyl-1-H-indol-5-yl)-1-(3,4,5-trimethoxyphenyl)propenone

E-2-Methyl-3-(1-methoxymethyl-1-H-indol-5-yl)-1-(3,4,5-trimethoxyphenyl)propenone

E-2-Methyl-3-(1-(N,N-dimethylaminoethyl)-1-H-indol-5-yl)-1-(3,4,5-trimethoxyphenyl)propenone

E-2-Methyl-3-(1-(N,N-dimethylaminopropyl)-1-H-indol-5-yl)-1-(3,4,5-trimethoxyphenyl)propenone

E-2-Methyl-3-[1-(2-pyrrolidinoethyl)-1-H-indol-5-yl]-1-(3,4,5-trimethoxyphenyl)propenone

2-Aminoacetic acid {2-methoxy-5-[2-methyl-3-oxo-3-(2,5-dimethoxyphenyl)propenyl]phenyl}amide

2-Aminopropanoic acid {2-methoxy-5-[2-methyl-3-oxo-3-(2,5-dimethoxyphenyl)propenyl]phenyl}amide

2-Amino-5-guanidinopentanoic acid {2-methoxy-5-[2-methyl-3-oxo-3-(2,5-dimethoxyphenyl)propenyl]phenyl}amide

2-Aminosuccinamic acid {2-methoxy-5-[2-methyl-3-oxo-3-(2,5-dimethoxyphenyl)propenyl]phenyl}amide

2-Aminosuccinic acid {2-methoxy-5-[2-methyl-3-oxo-3-(2,5-dimethoxyphenyl)propenyl]phenyl}amide

2-Amino-3-mercaptopropanoic acid {2-methoxy-5-[2-methyl-3-oxo-3-(2,5-dimethoxyphenyl)propenyl]phenyl}amide

2-Aminoglutamic acid {2-methoxy-5-[2-methyl-3-oxo-3-(2,5-dimethoxyphenyl)propenyl]phenyl}amide

2-Amino-3-methylpentanoic acid {2-methoxy-5-[2-methyl-3-oxo-3-(2,5-dimethoxyphenyl)propenyl]phenyl}amide

2,6-Diaminocaproic acid {2-methoxy-5-[2-methyl-3-oxo-3-(2,5-dimethoxyphenyl)propenyl]phenyl}amide

2-Amino-4-methylthiobutanoic acid {2-methoxy-5-[2-methyl-3-oxo-3-(2,5-dimethoxyphenyl)propenyl]phenyl}amide

2-Aminocaproic acid {2-methoxy-5-[2-methyl-3-oxo-3-(2,5-dimethoxyphenyl)propenyl]phenyl}amide

2-Amino-3-phenylpropanoic acid {2-methoxy-5-[2-methyl-3-oxo-3-(2,5-dimethoxyphenyl)propenyl]phenyl}amide

Pyrrolidine-2-carboxylic acid {2-methoxy-5-[2-methyl-3-oxo-3-(2,5-dimethoxyphenyl)propenyl]phenyl}amide

2-Amino-3-hydroxypropanoic acid {2-methoxy-5-[2-methyl-3-oxo-3-(2,5-dimethoxyphenyl)propenyl]phenyl}amide

2-Amino-3-hydroxybutanoic acid {2-methoxy-5-[2-methyl-3-oxo-3-(2,5-dimethoxyphenyl)propenyl]phenyl}amide

2-Amino-3-(1-H-indol-5-yl)propanoic acid {2-methoxy-5-[2-methyl-3-oxo-3-(2,5-dimethoxyphenyl)propenyl]-phenyl}amide

2-Amino-3-(4-hydroxyphenyl)propanoic acid {2-methoxy-5-[2-methyl-3-oxo-3-(2,5-dimethoxyphenyl)propenyl]phenyl}amide

2-Amino-2-methylbutanoic-{2-methoxy-5-[2-methyl-3-oxo-3-(2,5-dimethoxyphenyl)propenyl]phenyl}amide

2-(N-Methylamino)acetic acid {2-methoxy-5-[2-methyl-3-oxo-3-(2,5-dimethoxyphenyl)propenyl]phenyl}amide

2-Amino-5-[3-(1-nitroguanidino)-]-pentanoic acid {2-methoxy-5-[2-methyl-3-oxo-3-(2,5-dimethoxyphenyl)propenyl]phenyl}amide

1-methylpyrrolidine-2-carboxylic acid {2-methoxy-5-[2-methyl-3-oxo-3-(2,5-dimethoxyphenyl)propenyl]phenyl}amide

E-2-Methyl-3-(1-H-indol-5-yl)-1-(2,5-dimethoxyphenyl)propenone

E-2-Methyl-3-(1-methyl-1-H-indol-5-yl)-1-(2,5-dimethoxyphenyl)propenone;

E-2-Methyl-3-(2,3-dihydro-1-H-indol-5-yl)-1-(2,5-dimethoxyphenyl)propenone

E-2-Methyl-3-(1-methyl-2,3-dihydro-1-H-indol-5-yl)-1-(2,5-dimethoxyphenyl)propenone

E-2-Methyl-3-(1-hydroxymethyl-1-H-indol-5-yl)-1-(2,5-dimethoxyphenyl)propenone

E-2-Methyl-3-(1-hydroxyethyl-1-H-indol-5-yl)-1-(2,5-dimethoxyphenyl)propenone

E-2-Methyl-3-(1-methoxymethyl-1-H-indol-5-yl)-1-(2,5-dimethoxyphenyl)propenone

E-2-Methyl-3-(1-(N,N-dimethylaminoethyl)-1-H-indol-5-yl)-1-(2,5-dimethoxyphenyl)propenone

E-2-Methyl-3-(1-(N,N-dimethylaminopropyl)-1-H-indol-5-yl)-1-(2,5-dimethoxyphenyl)propenone

E-2-Methyl-3-[1-(2-pyrrolidinoethyl)-1-H-indol-5-yl]-1-(2,5-dimethoxyphenyl)propenone

2-Aminoacetic acid {2-methoxy-5-[2-methyl-3-oxo-3-(3-ethoxy-4,5-methylenedioxyphenyl)propenyl]phenyl}amide

2-Aminopropanoic acid {2-methoxy-5-[2-methyl-3-oxo-3-(3-methoxy-4,5-methylenedioxyphenyl)propenyl]phenyl}amide

2-Amino-5-guanidinopentanoic acid {2-methoxy-5-[2-methyl-3-oxo-3-(3-methoxy-4,5-methylenedioxyphenyl)-propenyl]phenyl}amide

2-Aminosuccinamic acid {2-methoxy-5-[2-methyl-3-oxo-3-(3-methoxy-4,5-methylenedioxyphenyl)propenyl]phenyl}amide

2-Aminosuccinic acid {2-methoxy-5-[2-methyl-3-oxo-3-(3-methoxy-4,5-methylenedioxyphenyl)propenyl]phenyl}amide

2-Amino-3-mercaptopropanoic acid {2-methoxy-5-[2-methyl-3-oxo-3-(3-methoxy-4,5-methylenedioxyphenyl)propenyl]phenyl}amide

2-Aminoglutamic acid {2-methoxy-5-[2-methyl-3-oxo-3-(3-methoxy-4,5-methylenedioxyphenyl)-propenyl]phenyl}amide

2-Amino-3-methylpentanoic acid {2-methoxy-5-[2-methyl-3-oxo-3-(3-methoxy-4,5-methylenedioxyphenyl)propenyl]-phenyl}amide

2,6-Diaminocaproic acid {2-methoxy-5-[2-methyl-3-oxo-3-(3-methoxy-4,5-methylenedioxyphenyl)propenyl]phenyl}amide

2-Amino-4-methylthiobutanoic acid {2-methoxy-5-[2-methyl-3-oxo-3-(3-methoxy-4,5-methylenedioxyphenyl)-propenyl]phenyl}amide

2-Aminocaproic acid {2-methoxy-5-[2-methyl-3-oxo-3-(3-methoxy-4,5-methylenedioxyphenyl)propenyl]phenyl}amide

2-Amino-3-phenylpropanoic acid {2-methoxy-5-[2-methyl-3-oxo-3-(3-methoxy-4,5-methylenedioxyphenyl)propenyl]-phenyl}amide

Pyrrolidine-2-carboxylic acid {2-methoxy-5-[2-methyl-3-oxo-3-(3-methoxy-4,5-methylenedioxyphenyl)propenyl]-phenyl}amide

2-Amino-3-hydroxypropanoic acid {2-methoxy-5-[2-methyl-3-oxo-3-(3-methoxy-4,5-methylenedioxyphenyl)propenyl]phenyl}amide

2-Amino-3-hydroxybutanoic acid {2-methoxy-5-[2-methyl-3-oxo-3-(3-methoxy-4,5-methylenedioxyphenyl)propenyl]-phenyl}amide

2-Amino-3-(1-H-indol-5-yl)-propanoic acid {2-methoxy-5-[2-methyl-3-oxo-3-(3-methoxy-4,5-methylenedioxy-phenyl)propenyl]phenyl}amide

2-Amino-3-(4-hydroxyphenyl)propanoic acid {2-methoxy-5-[2-methyl-3-oxo-3-(3-methoxy-4,5-methylenedioxy-phenyl)propenyl]phenyl}amide

2-Amino-2-methylbutanoic acid {2-methoxy-5-[2-methyl-3-oxo-3-(3-methoxy-4,5-methylenedioxyphenyl)propenyl]phenyl}amide

2-(N-Methylamino)acetic acid {2-methoxy-5-[2-methyl-3-oxo-3-(3-methoxy-4,5-methylenedioxyphenyl)propenyl]phenyl}amide

2-Amino-5-[3-(1-nitroguanidino)]pentanoic acid {2-methoxy-5-[2-methyl-3-oxo-3-(3-methoxy-4,5-methylenedioxyphenyl)propenyl]phenyl}amide

1-Methylpyrrolidine-2-carboxylic acid {2-methoxy-5-[2-methyl-3-oxo-3-(3-methoxy-4,5-methylenedioxyphenyl)-propenyl]phenyl}amide

E-2-Methyl-3-(1-H-indol-5-yl)-1-(3-methoxy-4,5-methylenedioxyphenyl)propenone

E-2-Methyl-3-(1-methyl-1-H-indol-5-yl)-1-(3-methoxy-4,5-methylenedioxyphenyl)propenone

E-2-Methyl-3-(2,3-dihydro-1-H-indol-5-yl)-1-(3-methoxy-4,5-methylenedioxyphenyl)propenone

E-2-Methyl-3-(1-methyl-2,3-dihydro-1-H-indol-5-yl)-1-(3-methoxy-4,5-methylenedioxyphenyl)propenone

E-2-Methyl-3-(1-hydroxymethyl-1-H-indol-5-yl)-1-(3-methoxy-4,5-methylenedioxyphenyl)propenone

E-2-Methyl-3-(1-hydroxyethyl-1-H-indol-5-yl)-1-(3-methoxy-4,5-methylenedioxyphenyl)propenone

E-2-Methyl-3-(1-methoxymethyl-1-H-indol-5-yl)-1-(3-methoxy-4,5-methylenedioxyphenyl)propenone

E-2-Methyl-3-(1-(N,N-dimethylaminoethyl)-1-H-indol-5-yl)-1-(3-methoxy-4,5-methylenedioxyphenyl)propenone

E-2-Methyl-3-(1-(N,N-dimethylaminopropyl)-1-H-indol-5-yl)-1-(3-methoxy-4,5-methylenedioxyphenyl)propenone

E-2-Methyl-3-[1-(2-pyrrolidinoethyl)-1-H-indol-5-yl]-1-(3-methoxy-4,5-methylenedioxyphenyl)propenone

Products according to the invention may be chosen from:

E-3-(3-Amino-4-methoxyphenyl)-2-methyl-1-(3,4,5-trimethoxyphenyl)propenone;

E-3-(4-Amino-3-methoxyphenyl)-2-methyl-1-(3,4,5-trimethoxyphenyl)propenone;

E-3-(3-Amino-4-methoxyphenyl)-2-methyl-1-(2,5-dimethoxyphenyl)propenone hydrochloride;

E-3-(3-Amino-4-methoxyphenyl)-2-bromo-1-(3,4,5-trimethoxyphenyl)propenone;

E-2-Methyl-3-(1-methyl-1-H-indol-5-yl)-1-(3,4,5-trimethoxyphenyl)propenone;

E-2-Methyl-3-(1-methyl-2,3-dihydro-1-H-indol-5-yl)-1-(3,4,5-trimethoxyphenyl)propenone;

E-2-Methyl-3-(1-methyl-1-H-indol-5-yl)-1-(3,4,5-trimethoxyphenyl)propenoneoxime;

E-2-Methyl-3-[1-(2-dimethylaminoethyl)-1-H-indol-5-yl]-1-(3,4,5-trimethoxyphenyl)propenone hydrochloride;

E-2-Methyl-3-(1-hydroxymethyl-1-H-indol-5-yl)-1-(3,4,5-trimethoxyphenyl)propenone;

E-2-Methyl-3-(1-methyl-1-H-indol-5-yl)-1-(3-methoxy-4,5-methylenedioxyphenyl)propenone;

E-2-Methyl-3-[1-(2-dimethylaminoethyl)-1-H-indol-5-yl]-1-(3-methoxy-4,5-methylenedioxyphenyl)propenone;

(S)-2,6-Diaminohexanoic acid {2-methoxy-5-[2-methyl-3-oxo-3-(3,4,5-trimethoxyphenyl)propenyl]phenyl}amide dihydrochloride;

2-Amino-5-[3-(1-nitroguanidino)]pentanoic acid {2-methoxy-5-[2-methyl-3-oxo-3-(3,4,5-trimethoxyphenyl)propenyl]phenyl}amide hydrochloride;

Aminoacetic acid {2-methoxy-5-[2-methyl-3-oxo-3-(3,4,5-trimethoxyphenyl)propenyl]phenyl}amide hydrochloride;

(S)-2-Amino-3-hydroxypropanoic acid {2-methoxy-5-[2-methyl-3-oxo-3-(3,4,5-trimethoxyphenyl)propenyl]phenyl}amide hydrochloride; and

1-Methylpyrrolidine-2-carboxylic acid {2-methoxy-5-[2-methyl-3-oxo-3-(3,4,5-trimethoxyphenyl)propenyl]}amide hydrochloride.

The products in accordance with the invention may be present in free or salified form, when they comprise at least one salifiable substituent. The products comprising at least one salifiable substituent may be salified. Examples of suitable salifiable substituents are amino, alkylamino, dialkylamino, imino, guanidino, hydrazino, imidazolino, pyrido, pyrimido and pyridazino substituents.

An example of a salified form is a hydrochloride.

A salified form of a product in accordance with the invention that has advantageous properties is (S)-2-amino-3-hydroxypropanoic acid {2-methoxy-5-[2-methyl-3-oxo-3-(3,4,5-trimethoxyphenyl)propenyl]phenyl}amide hydrochloride, having the following structure:

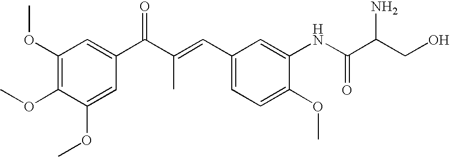

A product in accordance with the invention may be used for the manufacture of a medicinal product that is useful for treating a pathological condition, such as a cancer.

The present invention also relates to therapeutic compositions containing a compound according to the invention, in combination with an excipient that is pharmaceutically acceptable according to the chosen mode of administration. The pharmaceutical composition may be in solid or liquid form or in the form of liposomes.

Among the solid compositions that may be mentioned are powders, gel capsules and tablets. Among the oral forms that may also be included are solid forms protected against the acidic medium of the stomach. The supports used for the solid forms may be mineral supports, for instance phosphates or carbonates, or of organic supports, for instance lactose, celluloses, starch or polymers. The liquid forms may be solutions, suspensions or dispersions. They may contain as dispersive support either water or an organic solvent (ethanol, NMP or the like) or mixtures of surfactants and of solvents or complexing agents and solvents.

The liquid forms may be injectable and, as a result, will have a formulation that is acceptable for such a use.

Administration routes by injection that are acceptable include the intravenous, intraperitoneal, intramuscular and subcutaneous routes.

The administered dose of the compounds of the invention mayl be adapted by the doctor as a function of the route of administration for the patient and the patient's condition.

The compounds of the present invention may be administered alone or as a mixture with other anticancer agents. Among the possible combinations that may be mentioned are:

*alkylating agents such as cyclophosphamide, melphalan, ifosfamide, chlorambucil, busulfan, thiotepa, prednimustine, carmustine, lomustine, semustine, streptozotocin, decarbazine, temozolomide, procarbazine and hexamethylmelamine

*platinum derivatives such as cisplatin, carboplatin or oxaliplatin

*antibiotic agents such as bleomycin, mitomycin or dactinomycin

*antimicrotubule agents such as vinblastine, vincristine, vindesin, vinorelbin and taxoids (paclitaxel and docetaxel)

*anthracyclines such as doxorubicin, daunorubicin, idarubicin, epirubicin, mitoxantrone and losoxantrone

*group I and II topoisomerases such as etoposide, teniposide, amsacrine, irinotecan, topotecan and tomudex

*fluoropyrimidines such as 5-fluorouracil, UFT and floxuridine

*cytidine analogs such as 5-azacytidine, cytarabine, gemcitabine, 6-mercaptomurine and 6-thioguanine

*adenosine analogs such as pentostatin, cytarabine or fludarabine phosphate

*methotrexate and folinic acid

*various enzymes and compounds such as L-asparaginase, hydroxyurea, trans-retinoic acid, suramine, dexrazoxane, amifostine, herceptin and also estrogenic and androgenic hormones

*antivascular agents such as combretastatine derivatives or colchicine derivatives and prodrugs thereof.

It is also possible to combine the compounds of the present invention with a radiotherapy. These treatments may be administered simultaneously, separately or sequentially. The treatment may be adapted by the doctor as a function of the patient to be treated.

A product in accordance with the invention may promote the disintegration of lumps of cells originating from a vascular tissue. The products of the present invention may be used in their first therapeutic application to inhibit the growth of cancer cells and at the same time the destruction of existing vessels. The inhibition of vascularization is determined by a cell detachment test as described below.

Test for determining the inhibition of vascularization

A test to determine the detachment of endothelial cells was established in order to select the products on the basis of their "in vitro"activity. This test for determining the endothelial cell detachment is characterized in that the endothelial cells, inoculated in plates which are coated at the bottom with a binder, e.g., chosen from gelatin, fibronectin or vitronectin, after culturing, are supplemented with a medium containing the test compound, and the cells are then labeled with a fluorescent substance, the cells which become detached are removed by washing and the fluorescence of the remaining cells is counted in a fluorimeter.

This test consists in measuring the detachment of endothelial cells cultured on substrates based on a binder, e.g., chosen from fibronectin, vitronectin and gelatin. One day after inoculating the cells in plates containing 96 wells, for example, the culture medium is replaced with a medium containing the test compound in the absence of serum. The same preparation is prepared six times at three different concentrations (0.1, 0.3 and 0.6μM) and the control without addition of antivascular product is prepared six times. After treatment for two hours with the test substance, the cells are labeled with calcein-AM (1.6μg/ml) in a culture medium supplemented with 0.1% BSA. The cells which become detached are removed by washing with the culture medium containing 0.1% bovine serum albumin; 100μl of medium are added to each well. The fluoroscence of the remaining cells is counted in a fluorimeter. The data obtained are expressed relative to the control (untreated cells).

The assessment of the detachment of the endothelial cells in vitro is determined in the following way. HDMEC cells (Human Dermal Microvascular Endothelial Cells, Promocell, c-122102) are cultured in an ECGM-MV medium containing 5% fetal calf serum, growth factors (EGF 10hydrocortisone 1μg/ml, 0.4% growth supplement with heparin) and antibiotics (amphotericin 50 ng/ml, gentamicin 50μg/ml). For the detachment test, the HDMECs are inoculated at a rate of 5cells in clear-bottomed 96-well plates (Costar) precoated with fibronectin (10μg/ml) or vitronectin (1μg/ml) or gelatin. Twenty-four hours later, the culture medium is replaced with 0.1% BSA ECGM-MV medium containing the indicated products. The test concentrations are 0.1-0.3 and 1μM for each product. After treatment for two hours, the cells are labeled for 1 hour with calcein (1.6μg/ml, Molecular Probes) in 0.1% BSA ECGM-MV medium. The detached cells are then removed by washing with 0.1% BSA ECGM-MV medium; 100μl of medium are added to each well. The fluorescence of the cells which remain attached to the substratum of the well is counted using a fluorimeter, Spectrafluor Plus (Tecan, excitation at 485and emission at 535The data are the average of six different samples and are expressed as a percentage of the control (untreated cells).

A cell detachment effect of greater than or equal to 15% is considered as significant.

A product in accordance with the invention may be useful for inhibiting tubulin polymerization in vitro.

Assessment of the inhibition of tubulin polymerization

Tubulin is purified from pig brains according to the published methods (Shelanski et al., 1973, Proc. Natl, Acad. Sci. USA, 70, 765-768. Weingarten et al., 1975, Proc. Natl, Acad. Sci. USA, 72, 1858-1862). Briefly, the brains are ground and centrifuged in an extraction buffer. The tubulin, contained in the extract supernatant, is subjected to two successive cycles of polymerization at 37°C and depolymerization at 4°C, before being separated from the MAPs (Microtubule Associated Proteins) by chromatography on a phosphocellulose P11 column (Whatman). The tubulin thus isolated is more than 95% pure. It is stored in a buffer known as RB/2 30% glycerol, the composition of which is MES-NaOH [2-(N-morpholino)ethanesulfonic acid] 50 mM, pH 6.8; MgCl₂ 0.25 EGTA 0.5 30% glycerol (v/v), GTP (guanosine 5'-triphosphate) 0.2 mM.

The polymerization of the tubulin into microtubules is monitored by turbidimetry as follows; the tubulin is adjusted to a concentration of 10μM (1in the RB/2 30% glycerol buffer, to which is added 1mM GTP and 6mM MgCl₂. The polymerization is initiated by increasing the temperature from 6°C to 37°C in a cuvette with a 1optical pathlength, placed in a Uvikon 931 spectrophotometer (Kontron) equipped with a thermostatically-regulated cuvette holder. The increase in the turbidity of the solution is monitored at 350 nm

The products are dissolved at 10in DMSO and added at variable concentrations (0.5 to 10 μM) to the tubulin solution before polymerization. The IC₅₀ value is defined as the concentration of product which inhibits 50% of the rate of polymerization. A product whose IC₅₀ value is less than or equal to 3μM is considered as very active.

Assessment of the inhibition of proliferation of HeLa cells

The proliferation of HeLa cells is assessed by measuring the incorporation of [¹⁴C]-thymidine in the following way. The HeLa cells (epithelial tumor cells of human origin) are cultured in a DMEM medium (Gibco) which contains 10% fetal calf serum and antibiotics (1% penicillin, 1% streptomycin). To carry out the proliferation test, the cells are inoculated into 96-well cytostar microplates (Amersham), at a rate of 5cells per well. [¹⁴C]-thymidine (0.1μCi/well) and the products to be assessed are then added. Variable concentrations of products up to 10μM are used; the DMSO (solvent used to dissolve the products) should not exceed 0.5% in the medium. After incubation for 48at 37°C, the radioactivity incorporated into the cells is measured by counting the plate in a TRI-LUX counter (Wallac). The IC₅₀ value is defined as the concentration of product which reduces the radioactivity by 50% relative to an untreated control. A product whose IC₅₀ value is less than 1μM is considered as cytotoxic.

Assessment of the detachment effect on HDMEC endothelial cells

The assessment of the detachment of the endothelial cells in vitro is determined in the following way. HDMEC cells (Human Dermal Microvascular Endothelial Cells, Promocell, c-122102) are cultured in an ECGM-MV medium containing 5% fetal calf serum, growth factors (EGF 10hydrocortisone 1μg/ml, 0.4% growth supplement with heparin) and antibiotics (amphotericine 50 ng/ml, gentamicin 50μg/ml). For the detachment test, the HDMECs are inoculated at a rate of 5cells in clear-bottomed 96-well plates (Costar) precoated with fibronectin (10μg/ml) or vitronectin (1μg/ml) or gelatin. Twenty-four hours later, the culture medium is replaced with 0.1% BSA ECGM-MV medium containing the indicated products. The test concentrations are 0.1-0.3 and 1μM for each product. After treatment for two hours, the cells are labeled for 1 hour with calcein (1.6μg/ml, Molecular Probes) in 0.1% BSA ECGM-MV medium. The detached cells are then removed by washing with 0.1% BSA ECGM-MV medium; 100μl of medium are added to each well. The fluorescence of the cells which remain attached to the substratum of the well is counted using a fluorimeter, Spectrafluor Plus (Tecan, excitation at 485and emission at 535The data are the average of six different samples and are expressed as a percentage of the control (untreated cells).

A cell detachment effect of greater than or equal to 15% is considered as significant.

Assessment of the tumor necrosis in vivo

Mice are bred by IFFA-CREDO (Domaine des Oncins, 69210 L"Arbresle, France) from a race obtained by Jackson Laboratories, Bar Harbor, ME, USA, or by Charles River France (76410 St Aubin les Elbeuf, France) from a race obtained by Charles River, USA. The mice initially weigh more than 18 g at the start of the test. They have free access to food (UAR reference 113, Villemoisson, 91160 Epinay sur Orge, France) and water.

The tumors used are currently transplanted in our laboratories. All these tumors are at the Frederick Cancer Research Facility (Frederick, MD, USA) in the frozen tumor deposit of the National Cancer Institute (NCI) or at the American Type Culture Collection (ATCC, Rockville, MD, USA).

The tumor transplantation techniques, the chemotherapy and the data analysis are presented in detail (Corbett et al. 1982a; Corbett et al., 1982b).

To summarize, the animals required for an experiment are assembled and implanted bilaterally on day 0 (zero).

The growth of the solid tumors develops freely up to the desired size. The mice are then treated by intravenous injection of a test compound in solution.

The sampling of tumors is usually (but not necessarily) carried out 24 hours after the treatment.

The mice are killed by cerebral dislocation. The implanted tumors, along with the skin covering them and the neighboring tissues, are collected and stored in 10% formaldehyde (v/v) (Carlo Erba, Val de Reuil, France).

The samples are then treated, sectioned, stained with hematoxylin, eosin and saffron yellow, and are then examined macroscopically. The tumor necrosis (necrosis ± degeneration) is assessed microscopically using a scale of magnitude from 0 to 5:

0 = absence of necrosis;

1 = minimal, <5%;

2 = small, 5-25%;

3 = moderate, 25-50%;

4 = pronounced, 50-75%;

5 = large, >75%.

The values assigned to the tumor necrosis 24after administration of the test compound correspond solely to a necrosis dependent on the product which may be differentiated with certainty from any existing necrosis arising from the experiment.

The necrosis due to the experiment was assessed on an untreated control.

The tumor model is a C51 murine adenocarcinoma. This colon tumor is a grade III mucous colon adenoma. It is maintained by serial subcutaneous passages every 18 days in female BALB/c mice. The experiments were carried out on female BALB/c mice.

Results

Under the conditions as described, the following results were obtained for the examples described below:

- Example 1: selected dose 24.5- grade 5 necrosis

- Example 2: selected dose 50- grade 5 necrosis

References

CORBETT, T.H., LEOPOLD, W.R., DYKES D.J., ROBERTS, B.J., GRISWOLD, D.P., Jr and SCHABEL, F.M., Jr., Toxicity and anticancer activity of new triazine antifolate (NSC 127755). Cancer Res., 1982a, 42, 1707-1715.

CORBETT, T.H., ROBERTS, B.J., TRADER, M.W., LASTER, W.R., Jr., GRISWOLD, D.P., Jr and SCHABEL, F.M., Jr., Response of transplantable tumors of mice to anthracenedione derivatives alone and in combination with clinically useful agents. Cancer Treat. Rep., 1982b, 66, 1187-1200.

Definitions

"Halogen" is an element chosen from F, Cl, Br and I.

"C₁-C₇ linear alkyl" is a substituent chosen from methyl, ethyl, n-propyl, n-butyl, n-pentyl, n-hexyl and n-heptyl.

"C₁-C₇ branched alkyl"is a substituent chosen from 1-methylethyl, 1-methylpropyl, 2-methylpropyl, 1,1-dimethylethyl, 1-methylbutyl, 2-methylbutyl, 3-methylbutyl, 1,1-dimethylpropyl, 1,2-dimethylpropyl, 2,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1,1,2-trimethylpropyl, 1,2,2-trimethylpropyl, 1-ethyl-1-methylpropyl, 1-ethyl-2-methylpropyl, 1-ethylbutyl, 2-ethylbutyl, 1-methylhexyl, 2-methylhexyl, 3-methylhexyl, 4-methylhexyl, 5-methylhexyl, 1,1-dimethylpentyl, 1,2-dimethylpentyl, 1,3-dimethylpentyl, 1,4-dimethylpentyl, 2,2-dimethylpentyl, 2,3-dimethylpentyl, 2,4-dimethylpentyl, 3,3-dimethylpentyl, 3,4-dimethylpentyl, 4,4-dimethylpentyl, 1,1,2-trimethylbutyl, 1,1,3-trimethylbutyl, 1,2,2-trimethylbutyl, 1,2,3-trimethylbutyl, 1,3,3-trimethylbutyl, 2,2,3-trimethylbutyl, 2,3,3-trimethylbutyl, 1,1,2,2-tetramethylpropyl, 1-ethylpentyl, 2-ethylpentyl, 3-ethylpentyl, 1-ethyl-1-methylbutyl, 1-ethyl-2-methylbutyl, 1-ethyl-3-methylbutyl, 2-ethyl-1-methylbutyl, 2-ethyl-2-methylbutyl, 2-ethyl-3-methylbutyl, 1-propylbutyl, 1-(1-methylethyl)butyl, 1-(1-methylethyl)-2-methylpropyl.

"Cycloalkyl" may be a substituent chosen from cyclopropyl, cyclobutyl, cyclopentyl, cyclohexyl, cycloheptyl and bicyclo[2.2.1]heptyl.

"Aryl" may be a substituent chosen from phenyl, naphthyl, pyridyl, quinoleyl, isoquinoleyl, pyrimidyl, piperazinyl, triazinyl, carbazolyl, imidazolyl, thiazolyl, oxazolyl, benzimidazolyl, benzothiazolyl, benzoxazolyl, triazolyl, tetrazolyl, benzotriazolyl, thienyl, furyl, pyrolyl, benzothienyl, benzofuryl and indolyl.

"Aralkyl" is an alkyl substituent, itself substituted with an aryl group as defined above. A benzyl group is an example of an aralkyl substituent.

The term "substituted" that is especially present in the expressions "substituted C₁-C₇ linear alkyl", "substituted C₁-C₇ branched alkyl", "substituted cyclo alkyl"and "substituted aralkyl"necessarily relates to a substituent other than H, which may be chosen from F, Cl, Br, I, N(R7, R8), N(O)(R7, R8), NO, NO₂, O(R7), S(R7), SO(R7), SO₂(R7), OSO₂(R7), PO₃(R7), OPO₃(R7), CO(R7), COO-(R7), CONH-(R7), CON(R7, R8), CN, C≡C-(R7), N=C-(R9), aryl, aralkyl, heteroaryl and heteroaralkyl; in which R7, R8 are independently selected from the group consisting of H, alkyl, alkylene, aryl, heteroaryl, aralkyl, C(O)-(R7), C(S)-(R7), alkyl-O(R7), alkyl-S(R7) and alkyl-N(R7, R8), in which, when R7 and R8 are simultaneously present, they may be linked together to form a ring, R9 is selected from the group consisting of alkyl, alkylene, aryl, heteroaryl, aralkyl, alkyl-O(R7), alkyl-S(R7) and alkyl-N(R7, R8); and to any other acceptable substituent known to those skilled in the art.

"Amino acid" comprises the natural and artificial amino acids, in enantiomerically pure form or as a mixture.

"Metabolizable substituent" includes any substituent containing at least one functional group that can be cleaved by metabolism of a living being. Examples of cleavable functional groups include amides, imides, imines, esters, lactones, lactams, acetals, hemiacetals, carbonates, carbamates and ureas.

"Organic acid" includes organic molecules containing at least one proton-donating functional group, for example COOH, SO₃H, OSO₃H, PO₃H₂, OPO₃H₂, and optionally OH when the latter functional group is directly linked to an aromatic or heteroaromatic nucleus.

"Mineral acid" includes proton (H⁺) -donating mineral products, for example HCl, HBr, HI, HIO₄, H₂S, H₂SO₄, H₃PO₂, H₃PO₄ and HNO₃; or species capable of giving mineral acids by reaction, for example AlCl₃, AlBr₃, SnCl₄, SiCl₄, TiCl₄, FeCl₃ or RuCl₃ which may react in a protic solvent such as water according to:

MₐZ_{b} + bH₂O → a{M(OH)_{b} and/or [MOₙ(OH)_{b-2n} + nH₂O]} + bHZ

with a, b and n being whole or real stoichiometric coefficients, M chosen from the metals given above as examples, and Z is a halogen.

In the text hereinbelow, the products corresponding to Formula (I) will be represented by a general formula (I) in which Ar is replaced with a phenyl substituted with n methoxy groups, it being understood that n takes the value 2, 3 or 4 and that the position of the methoxy groups on the phenyl is as defined above.

The chalcones of general formula (I)

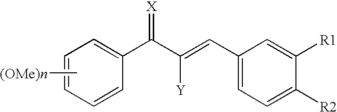

in which X represents an oxygen atom, Y is other than a halogen atom and n, R₁ and R₂ are defined as above with R₁ or R₂ representing an amino radical, may be prepared by coupling between an aromatic ketone of general formula (II) in which Y is other than a halogen atom, and an aromatic aldehyde of general formula (III) under the conditions described in J.Chem., 1990, 33, 1948. This coupling is followed by reduction of the nitro radical to an amino radical according to scheme (I):

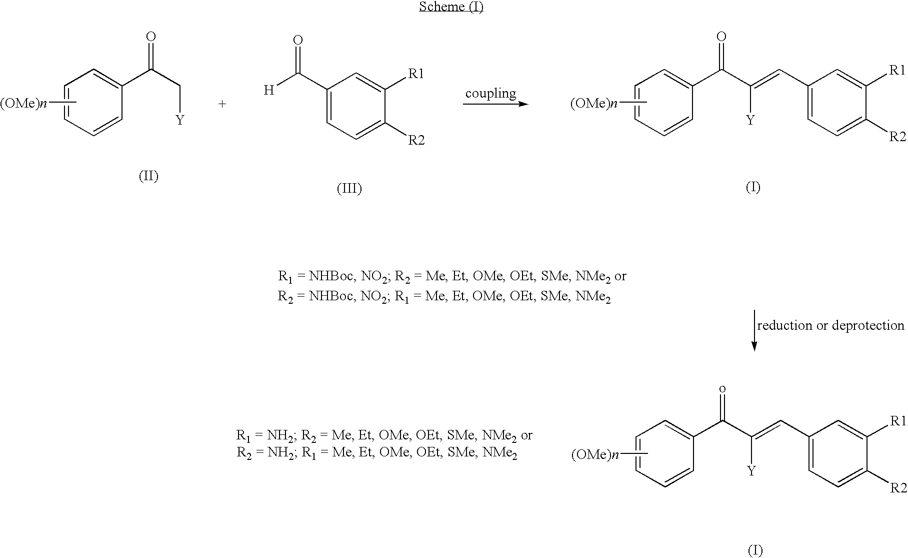

The process is generally performed in apparatus of Soxhlet type at the reflux point of an alcohol, such as ethanol, in the presence of piperidine, acetic acid and molecular sieves.

It is understood that the coupling between the ketone of general formula (II) and the aldehyde of general formula (III) may also be carried out with a radical R₁ or R₂ in which the nitro radical is replaced with an amino radical. It is understood that the coupling may also be carried out with the radical R₁ or R₂ representing any protected form of the aromatic amine function, such as, by way of nonlimiting example, tert-butyloxycarbonylamino (NHBoc). The cleavage of the protecting group on the aromatic amine function may be carried out under the conditions described in Protective Groups in Organic Chemistry (edited by Wiley). In the particular case of the reduction of the nitro radical to an amino radical, it is possible to use catalytic reduction methods, for instance hydrogen in the presence of a catalyst such as 3% palladium-on-charcoal, or chemical reduction, for instance iron in the presence of hydrochloric acid or stannous chloride.

The aromatic aldehydes of general formula (III) are either commercially available or previously described in the literature.

The aromatic ketones of general formula (II) are described in the literature and generally prepared from the corresponding aromatic aldehydes which are commercially available. When Y represents an optionally substituted alkyl or aralkyl radical, the process may be performed by reacting the aldehyde with a suitably chosen organometallic reagent, followed by oxidizing the benzyl alcohol thus obtained under the conditions described in J. Med. Chem., 1990, 33, 1948. When Y represents a carboxyl, carboxylate or carboxamide radical, the aldehyde may be reacted with a diazoacetate under the conditions described in Synlett, 1996, 369.

The chalcones of general formula (I)

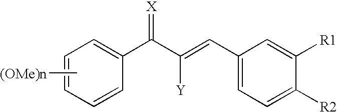

in which X represents an oxygen atom, Y represents a halogen atom, such as a bromine or chlorine atom, and n, R₁ and R₂ are defined as above with R₁ or R₂ representing an amino, may be prepared by addition of halogen followed by dehydrohalogenation of a chalcone in which Y represents a hydrogen atom, according to scheme (II). This addition-elimination sequence may be performed on a protected form of the amino radical, such as the nitro or NHBoc radical, and then reduced or deprotected.

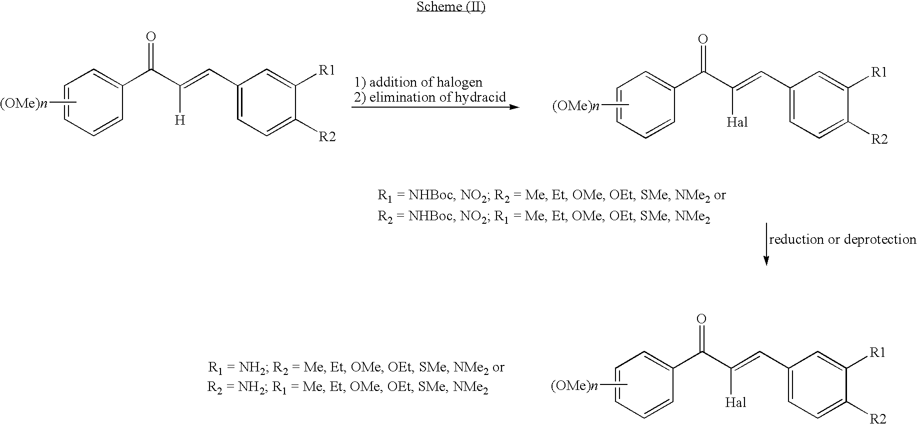

The addition of halogen, such as bromine or chlorine, is generally carried out in a solvent such as chloroform or carbon tetrachloride at a temperature of between 0 and 50°C. The dehydrohalogenation is generally carried out in a solvent such as dichloromethane in the presence of an organic or mineral base, for instance triethylamine, sodium hydroxide or potassium carbonate, at a temperature of between 0°C and the reflux point of the reaction medium.

The chalcones of general formula (I)

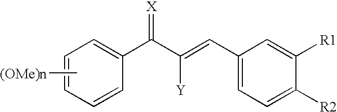

in which X represents an oxygen atom, Y is other than a halogen atom and n, R₁ and R₂ are defined as above with R₁ or R₂ representing an amino radical, may also be prepared by nucleophilic displacement of a product of general formula (I) in which Y represents a halogen atom, such as a bromine atom. In this case, the ketone function may be protected beforehand according to the general scheme (III):

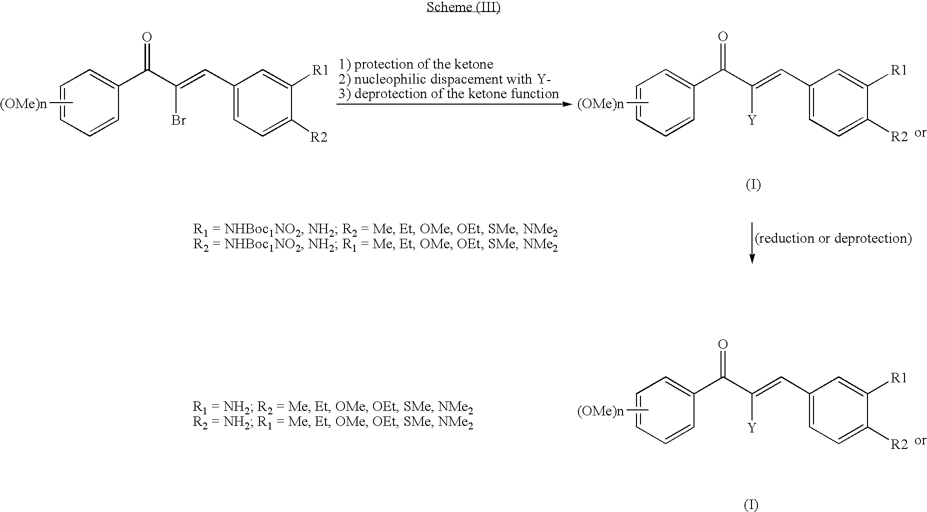

The chalcones of general formula (I)

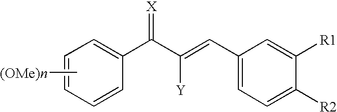

in which X represents an oxygen atom, Y represents a substituted methylene radical and n, R₁ and R₂ are defined as above with R₁ or R₂ representing an amino radical, may also be prepared by nucleophilic displacement of a product of general formula (I) in which Y represents a bromomethyl radical, under the conditions described in J. Org. Chem., 1967, 3830, according to the general scheme (IV):

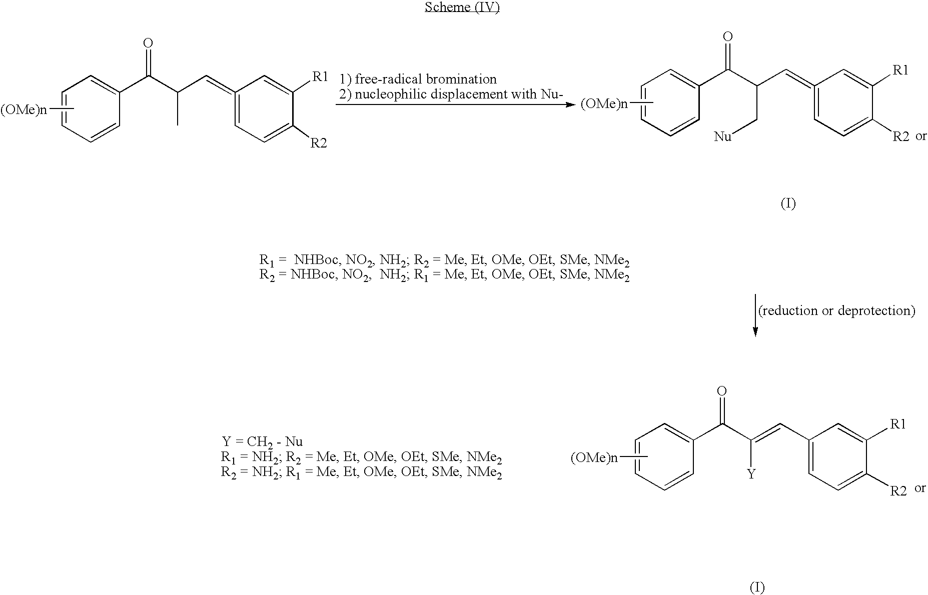

Schemes (III) and (IV) illustrate, in a nonlimiting manner, methods for modifying the substituents Y on a preformed chalcone, it being possible for any other method known to those skilled in the art to be used to modify said substituent Y.

The chalcones of general formula (I)

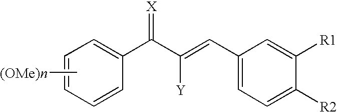

in which X represents a radical N-OR₃ and Y, n, R₁ and R₂ are defined as above with R₁ or R₂ representing an amino, may be prepared by the action of a hydroxylamine on a product of general formula (I) in which X represents an oxygen atom, according to scheme (V):

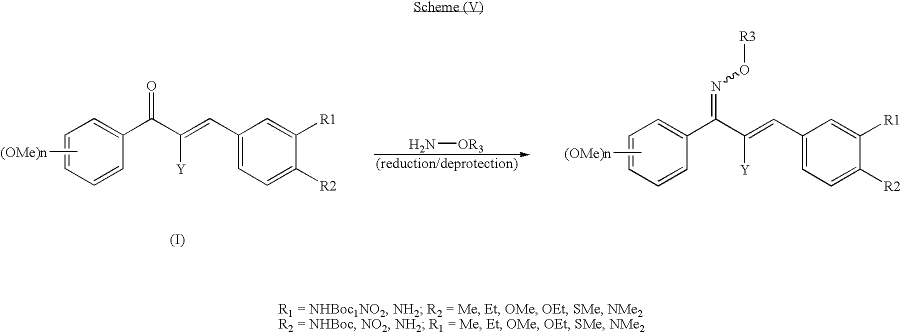

It is understood that the present invention also relates to prodrugs, in particular to water-soluble prodrugs, of the chalcones of general formula (I)

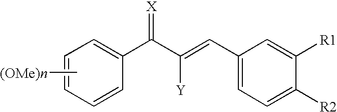

in which X represents an oxygen atom or a radical N-OR₃, and Y, n, R₁ and R₂ are defined as above with R₁ or R₂ representing a cleavable derivative of the amino radical. The cleavable derivatives of the amino radical include amino acid derivatives which may be prepared by coupling of peptide type between

(i)a product of general formula (I) in which X represents an oxygen atom or a radical N-OR₃, in which Y, n, R₁ and R₂ are defined as above with R₁ or R₂ representing an amino radical, and

(ii)a natural or modified amino acid, optionally in protected form, with the exception of its carboxylic function, according to scheme (VI), it being understood that when the amino acid is partially protected, the coupling is followed by a deprotection step:

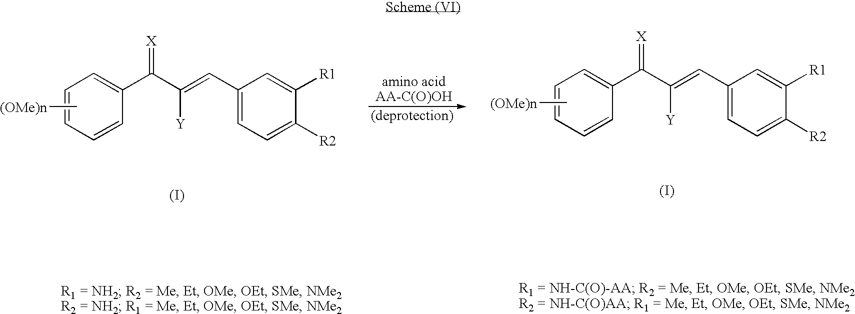

The coupling of peptide type is carried out under standard conditions, in an organic solvent, such as dichloromethane, in the presence of a coupling and/or activating agent such as, by way of nonlimiting example, the EDCI/HOBT mixture.

The examples which follow are given by way of illustration of the invention.

Example 1 E-3-(3-Amino-4-methoxyphenyl)-2-methyl-1-(3,4,5-trimethoxyphenyl)propenone

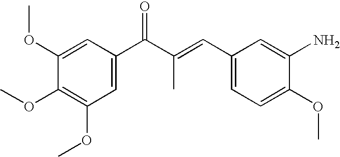

Step 1: 2.24g of 1-(3,4,5-trimethoxyphenyl)propenone, which may be prepared according to Biorg. Med. Chem. 1998, 8(9), 1051, 1.85of 3-nitro-4-methoxybenz2 mL of piperidine and 1of acetic acid in 20of ethanol were successively added to a 25three-necked flask on which was mounted a Soxhlet filled with 3Åmolecular sieves. The medium was refluxed for 48After cooling, the reaction medium was concentrated under reduced pressure and then taken up in 100of ethyl acetate, and the organic phase was washed with water, dried over magnesium sulfate and concentrated under reduced pressure. The crude product was purified by flash chromatography on silica gel (70-230 mesh), eluting with a mixture of cyclohexane and ethyl acetate (80/20 by volume). 2 g of E-3-(3-nitro-4-methoxyphenyl)-2-methyl-1-(3,4,5-trimethoxywere thus obtained, containing about 5% of the Z isomer, in the form of a yellow solid with a melting point of between 45 and 50°C.

Step 2: 485 mg of 3-(3-nitro-4-methoxyphenyl)-2-methyl-1-(3,4,5-trimethoxyphenyl)propenone, obtained in the preceding step, were placed in suspension in 20 mL of ethanol and 2.5 mL of water in a 50 mL three-necked flask. The mixture was brought to reflux and 0.25 mL of 37% hydrochloric acid was then added, followed by portionwise addition of 2.09 g of iron filings. The reflux was maintained for 30 minutes and the mixture was then cooled. After addition of 2 g of potassium carbonate, the insoluble materials were filtered and washed with 3 times 25 mL of ethanol. After concentrating the filtrates under reduced pressure, the residue was taken up in 50 mL of water and extracted with 3 times 50 mL of ethyl acetate. The combined organic phases were washed with water, dried over magnesium sulfate and concentrated under reduced pressure. The orange-colored residue thus obtained was purified by flash chromatography on silica gel (70-230 mesh), eluting with a mixture of cyclohexane and ethyl acetate (70/30 by volume). 0.35 g of pure E-3-(3-amino-4-methoxyphenyl)-2-methyl-1-(3,4,5-trimethoxyphenyl)propenone was thus obtained in the form of a pale yellow powder, the characteristics of which were as follows:

- melting point (Kofler) = 142°C

- elemental analysis: %C = 67.26; %H = 6.80; %N = 3.97.

Example 2: E-3-(4-Amino-3-methoxyphenyl)-2-methyl-1-(3,4,5-trimethoxyphenyl)propenone :

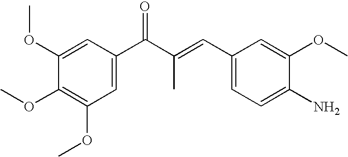

Step 1: Working as in step 1 of example 1, but starting with 2.24 g of 1-(3,4,5-trimethoxyphenyl)propenone and 1.81 g of 4-nitro-3-methoxybenzaldehyde in 75 mL of ethanol containing 2 mL of piperidine and 1 mL of acetic acid, by refluxing for 96 hours, and after purification by flash chromatography on silica gel (70-230 mesh) eluting with a mixture of cyclohexane and ethyl acetate (80/20 by volume), 0.7 g of E-3-(4-nitro-3-methoxyphenyl)-2-methyl-1-(3,4,5-trimethoxyphenyl)propenone free of Z isomer was obtained in the form of a very viscous yellow oil.

Step 2: Working as in step 2 of example 1, but starting with 700 mg of 3-(4-nitro-3-methoxyphenyl)-2-methyl-1-(3,4,5-trimethoxyphenyl)propenone and 3.09 g of iron, and after purification by flash chromatography on silica gel (70-230 mesh) eluting with a mixture of cyclohexane and ethyl acetate (70/30 by volume), 0.55 g of pure E-3-(4-amino-3-methoxyphenyl)-2-methyl-1-(3,4,5-trimethoxyphenyl)propenone was obtained in the form of a pale yellow powder, the characteristics of which were as follows:

- melting point (Kofler) = 140°C

- elemental analysis: %C = 67.27; %H = 6.68; %N = 3.93.

Example 3 : E-3-(3-Amino-4-methoxyphenyl)-2-methyl-1-(2,5-dimethoxyphenyl)propenone hydrochloride

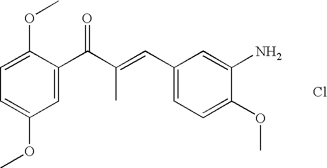

Step 1: Working as in step 1 of example 1, but starting with 3.8 g of 1-(2,5-dimethoxyphenyl)propenone and 3.7 g of 3-nitro-4-methoxybenzaldehyde in 75 mL of ethanol containing 4 mL of piperidine and 2 mL of acetic acid, by refluxing for 96and after purification by flash chromatography on silica gel (70-230 mesh) eluting with a mixture of cyclohexane and ethyl acetate (80/20 by volume), followed by recrystallization from isopropyl acetate, 0.3 g of E-3-(3-nitro-4-methoxyphenyl)-2-methyl-1-(2,5-dimethoxyphenyl)propenone free of Z isomer, was obtained in the form of yellow crystals melting at 104°C.

Step 2: Working as in step 2 of example 1, but starting with 280 mg of 3-(3-nitro-4-methoxyphenyl)-2-methyl-1-(2,5-dimethoxyphenyl)propenone and 1.03 g of iron, and after purification in the form of the hydrochloride recrystallized from a mixture of ethanol and diethyl ether, 0.25 g of pure E-3-(3-amino-4-methoxyphenyl)-2-methyl-1-(2,5-dimethoxyphenyl)propenone hydrochloride was obtained in the form of pale yellow crystals, the characteristics of which were as follows:

- melting point (Kofler) = 178°C

- elemental analysis: %C = 62.47; %H = 6.01; %N = 3.77; %Cl = 9.14.

Example 4 : E-3-(3-Amino-4-methoxyphenyl)-2-bromo-1-(3,4,5-trimethoxyphenyl)propenone

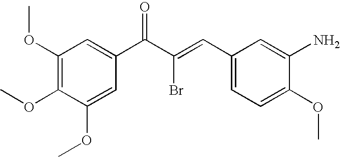

Step 1: Working as in step 1 of example 1, but starting with 3.8 g of 3,4,5-trimethoxyacetophenone and 3.44 g of 3-nitro-4-methoxybenzaldehyde in 95 mL of methanol containing 2.37 mL of sodium hydroxide, overnight at room temperature, and after purification by flash chromatography on silica gel (70-230 mesh) eluting with a mixture of cyclohexane and ethyl acetate (80/20 by volume), 6.27 g of E-3-(3-nitro-4-methoxyphenyl)-1-(3,4,5-trimethoxyphenyl)propenone free of Z isomer, were obtained in the form of a viscous yellow oil.

Step 2: 500 mg of E-3-(3-nitro-4-methoxyphenyl)-1-(3,4,5-trimethoxyphenyl)propenone were dissolved in 8of chloroform in a 50 mL three-necked flask. 40 μL of bromine dissolved in 3 mL of chloroform were then added dropwise. After stirring for 3 hours at room temperature, a further 40 μL of bromine dissolved in 3 mL of chloroform were added dropwise and stirring was continued for a further 3 hours at room temperature. After concentration under reduced pressure, the residue was purified by flash chromatography on silica gel (70-230 mesh), eluting with a mixture of dichloromethane and cyclohexane (50/50 by volume). 554 mg of 2,3-dibromo-3-(3-nitro-4-methoxyphenyl)-1-(3,4,5-trimethoxyphenyl)propanone were thus obtained, the characteristic of which was as follows:

- mass spectrum (EI/DCI) M⁺ = 533.

Step 3: 153 mg of 2,3-dibromo-3-(3-nitro-4-methoxyphenyl)-1-(3,4,5-trimethoxyphenyl)propanone were dissolved in 6 mL of dichloromethane dried over 4Åmolecular sieves, in a 10 mL three-necked flask, followed by addition of 81 μL of anhydrous triethylamine, and the mixture was stirred at room temperature for 24 hours. A further 40 μL of anhydrous triethylamine were then added and the mixture was stirred for a further 72 hours at room temperature. After addition of 5 mL of distilled water, the organic phase was separated out by settling of the phases, washed with twice 5 mL of water, dried over magnesium sulfate and concentrated under reduced pressure. 116 mg of E-2-bromo-3-(3-nitro-4-methoxyphenyl)-1-(3,4,5-trimethoxyphenyl)propenone were thus obtained, containing about 10% of its Z isomer, which product was used without further purification in the rest of the synthesis.

Step 4: 67.2of E-2-bromo-3-(3-nitro-4-methoxyphenyl)-1-(3,4,5-trimethoxyphenyl)propenone were placed in suspension in 2 mL of ethanol, 167.5 mg of stannous chloride, in its dihydrate form, were then added and the mixture was heated at 80°C for 1After cooling and diluting with 2 mL of water, the pH was brought to 8 by addition of saturated sodium hydrogen carbonate solution and the resulting mixture was extracted 3 times with 5of ethyl acetate. The organic phase was washed with water, dried over magnesium sulfate and concentrated under reduced pressure. The crude product was purified by flash chromatography on silica gel (70-230 mesh) eluting with dichloromethane. 34.4 mg of E-2-bromo-3-(3-amino-4-methoxyphenyl)-1-(3,4,5-trimethoxyphenyl)propenone, free of the Z isomer, were thus obtained, the characteristics of which were as follows:

- chromatography on a silica plate: Rf = 0.16 (solvent: dichloromethane)

- mass spectrum (EI/DCI) M⁺ = 422.

Example 5 : E-2-Methyl-3-(1-methyl-1-H-indol-5-yl)-1-(3,4,5-trimethoxyphenyl)propenone

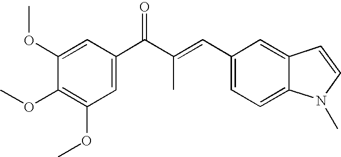

The process was performed as in step 1 of example 1, but starting with 4.49 g of 1-(3,4,5-trimethoxyphenyl)propanone and 3.18 g of 1-methylindole-5-carboxaldehyde - which may be prepared according to Terent"ew et al., J. Gen. Chem USSR (1962), 32, 1311 - in 100 mL of ethanol containing 4 mL of piperidine and 2 mL of acetic acid, by refluxing for 48 hours. After purification by flash chromatography on silica gel (70-230 mesh), eluting with a mixture of cyclohexane and ethyl acetate (80/20 by volume), followed by recrystallization from ethanol, 2.5 g of pure E-2-methyl-3-(1-methyl-1-H-indol-5-yl)-1-(3,4,5-trimethoxyphenyl)propenone were obtained in the form of pale yellow crystals, the characteristics of which were as follows:

-melting point (Kofler) = 136°C.

-elemental analysis: % C = 72.52 ; %H = 6.45; % N = 3.87.

Example 6 : E-2-Methyl-3-(1-methyl-2,3-dihydro-1-H-indol-5-yl)-1-(3,4,5-trimethoxyphenyl)propenone

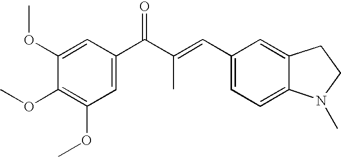

The process was performed as in step 1 of example 1, but starting with 4.49 g of 1-(3,4,5-trimethoxyphenyl)propenone and 3.22 g of 1-methyl-2,3-dihydroindole-5-carboxaldehyde - which may be prepared according to Gavinecki et al., Org. Prep. Proced. Int. (1998), 30, 455-60 - in 100 mL of ethanol containing 4of piperidine and 2 mL of acetic acid, by refluxing for 48 hours. After purification by flash chromatography on silica gel (70-230 mesh), eluting with a mixture of cyclohexane and ethyl acetate (70/30 by volume), followed by crystallization from isopropyl ether, 1.2 g of pure E-2-methyl-3-(1-methyl-2,3-dihydro-H-indol-5-yl)-1-(3,4,5-trimethoxyphenyl)-propenone were obtained in the form of pale yellow crystals, the characteristics of which were as follows:

-melting point (Kofler) = 85°C.

-elemental analysis : % C = 71.91; %H = 7.04; % N = 3.79.

Example 7 : E-2-Methyl-3-(1-methyl-1-H-indol-5-yl)-1-(3,4,5-trimethoxyphenyl)propenoneoxime

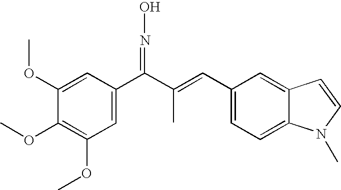

100 mg of E-2-methyl-3-(1-methyl-1-H-indol-5-yl)-1-(3,4,5-trimethoxyphenyl)propenone, obtained in example 5, 29 mg of hydroxylamine hydrochloride, 0.5 mL of piperidine and 5 mL of ethanol were placed in a 25 mL three-necked flask. The mixture was refluxed with stirring for 5 hours and stirring was then continued at room temperature for 20 hours. After concentration under reduced pressure, the reaction medium was taken up in 20 mL of water and 25 mL of of ethyl acetate. The organic phase was separated out by settling, washed with water and concentrated to dryness under reduced pressure. After purification by flash chromatography on silica gel (70-230 mesh), eluting with a mixture of cyclohexane and ethyl acetate (80/20 by volume), followed by recrystallization from ethanol, 70 mg of a 60/40 mixture of the Z and E isomers of E-2-methyl-3-(1-methyl-1-H-indol-5-yl)-1-(3,4,5-trimethoxyphenyl)propenoneoxime were obtained in the form of white crystals, the characteristics of which were as follows:

-melting point (Kofler) = 150°C.

-elemental analysis: % C = 68.63; %H = 6.30; % N = 7.03.

Example 8 : E-2-Methyl-3-(1-methyl-1-H-indol-5-yl)-1-(3,4,5-trimethoxyphenyl)propenone Z-oxime

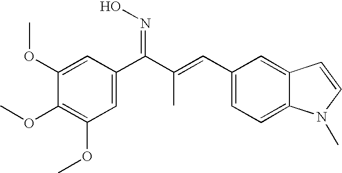

820 mg of the 60/40 mixture of the Z and E isomers of E-2-methyl-3-(1-methyl-1-H-indol-5-yl)-1-(3,4,5-trimethoxyphenyl)propenoneoxime, prepared as in example 7, were separated on a chiral column of Whelk01 S,S 10 µM type, containing 700 g of chiral stationary phase, eluting with a mixture of n-heptane/ethanol/dichloromethane (68/2/30 by volume) containing 0.1% trifluoroacetic acid at a flow rate of 100By isolating the first fraction eluted (retention time = 22 minutes), 434 mg of E-2-methyl-3-(1-methyl-1-H-indol-5-yl)-1-(3,4,5-trimethoxyphenyl)propenone Z-oxime were obtained.

Example 9 : E-2-Methyl-3-(1-methyl-1-H-indol-5-yl)-1-(3,4,5-trimethoxyphenyl)propenone E-oxime

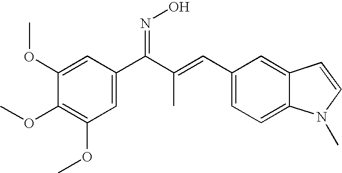

By working as in example 8, but isolating the second fraction eluted (retention time = 28 minutes), 328 mg of E-2-methyl-3-(1-methyl-1-H-indol-5-yl)-1-(3,4,5-trimethoxyphenyl)propenone E-oxime were obtained.

Example 10 : (S)-2,6-Diaminohexanoic acid {2-methoxy-5-[2-methyl-3-oxo-3-(3,4,5-trimethoxyphenyl)propenyl]-phenyl}amide dihydrochloride

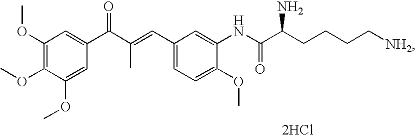

Step 1: 1 g of Boc-Lys(Boc)-OH·DCHA was dissolved in 10 ml of ethyl acetate in a 50round-bottomed flask, followed by addition of 1.2 equivalents of aqueous 2M sulfuric acid solution (i.e. 2.2 ml). Two clear phases were obtained. The organic phase was set aside. 5 ml of cold distilled water were added to the aqueous phase and the resulting mixture was then extracted with 2 x 5 ml of ethyl acetate. The organic phases were combined and washed with 2 x 10 ml of distilled water and then dried over magnesium sulfate and the solvent was evaporated off on a rotary evaporator (bath temperature below 40°C). The colorless oil was dried under vacuum to give 0.47 g of Boc-Lys(Boc)-OH.

Step 2: The Boc-Lys(Boc)-OH (458 mg, 1.319 mmol) prepared in step 1 was dissolved in 7 ml of ethyl acetate in a 50 ml three-necked flask equipped with a thermometer and a bubble counter. The colorless solution was cooled to 6°C (water bath + ice), followed by addition of N-methylmorpholine (1.2 equivalents, 146.5 µl), followed by pivaloyl chloride (1.2 equivalents, 163 µl). The white suspension obtained was maintained at 5°C for 2 hours 30 minutes and 3-(3-amino-4-methoxyphenyl)-2-methyl-1-(3,4,5-trimethoxyphenyl)propenone (392.9 mg, 1 equivalent) suspended in 10 ml of ethyl acetate was then added. The suspension was stirred at room temperature for 72 hours. The suspension was filtered through a sinter funnel and the solid was then rinsed with ethyl acetate. The filtrate was washed with 10 ml of distilled water and then with 5 ml of aqueous 1N sodium hydroxide solution, 10of distilled water and 10of saturated NaCl solution. The organic phase was dried over magnesium sulfate and the solvent was evaporated off on a rotary evaporator. The crude reaction product thus obtained was taken up in 5 ml of ethyl acetate and 3 ml of absolute ethanol, followed by addition of 1 ml of 4.8N hydrochloric ethanol. The medium was heated at 49°C until the expected product was obtained, the reaction progress being monitored by LC/MS. On cooling, a white solid precipitates out. The solid was filtered on a sinter funnel and washed with ethyl acetate. 299.3 mg of a pulverulent white solid were obtained, the characteristics of which were as follows:

- LC/MS (50x4.6 mm column of Hypersil BDS C18 3 µm silica; linear elution gradient from 5% to 90% acetonitrile, containing 0.05% trifluoroacetic acid, in water, also containing 0.05% trifluoroacetic acid, in 3.5 minutes at a flow rate of 1 mL/minute); retention time = 2.67 minutes; MS: 486.3 ([M+H]⁺)

- Elemental analysis: %C = 52.06; %H = 6,73; %N = 7.02; % Cl = 12.7.

Example 11 : 2-Amino-5-[3-(1-nitroguanidino)]-pentanoic acid {2-methoxy-5-[2-methyl-3-oxo-3-(3,4,5-trimethoxy-phenyl)propenyl]phenyl}amide hydrochloride

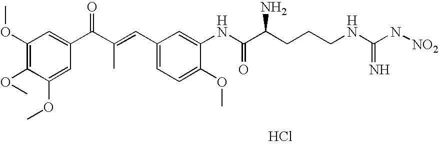

Step 1: 3-(3-Amino-4-methoxyphenyl)-2-methyl-1-(3,4,5-trimethoxyphenyl)propenone (1 g, 0.279 mmol) was dissolved in 150 ml of dichloromethane, followed by addition of EEDQ (760.1 mg, 1.1 equivalents) and Na-Boc-N-w-nitro-L-arginine (980.2 mg, 1.1 equivalents). The suspension was stirred at room temperature for 20 hours. The solution obtained was concentrated under vacuum on a rotary evaporator. The crude reaction product was purified by flash chromatography on silica gel (AC.C 35-70 µm silica 60), eluting with a mixture of ethyl acetate and cyclohexane (80/20 by volume). 1.5 g of 2-(1,1-dimethylethyloxycarbonylamino)-5-[3-(1-nitroguanidino)]pentanoic acid {2-methoxy-5-[2-methyl-3-oxo-3-(3,4,5-trimethoxyphenyl)propenyl]phenyl}amide were obtained in the form of a brown oil.

Step 2: This oil was dissolved in 10 ml of ethyl acetate, followed by addition of 4 ml of ethanol and 2 ml of 4.8N hydrochloric ethanol solution. The medium was heated at 60°C (temperature of the oil bath) for 12 hours. The white solid obtained was filtered off on a sinter funnel, and then rinsed with ethyl acetate and dried under vacuum. 1.07 g of 2-amino-5-[3-(1-nitroguanidino)]pentanoic acid {2-methoxy-5-[2-methyl-3-oxo-3-(3,4,5-trimethoxyphenyl)propenyl]phenyl}amide hydrochloride were thus obtained in the form of a white powder, the characteristics of which were as follows:

- Mass spectrum (EI): m/z = 558

- Elemental analysis: %C = 52,84 ; %H = 6,22 ; %N = 14,14; %Cl = 6,12.

Example 12 : 1-Methylpyrrolidine-2-carboxylic acid {2-methoxy-5-[2-methyl-3-oxo-3-(3,4,5-trimethoxyphenyl)propenyl]phenyl}amide hydrochloride

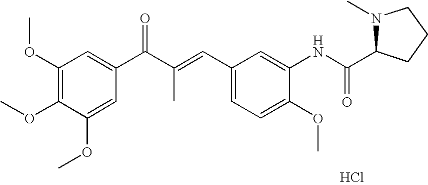

3-(3-Amino-4-methoxyphenyl)-2-methyl-1-(3,4,5-trimethoxyphenyl)propenone (450 mg, 1.259 mmol) was dissolved in 45 ml of DMF, followed by addition of HOBT (578.3 mg, 3 equivalents), HBTU (1.43 g, 3 equivalents), N-methyl-L-proline (487.9 mg, 3 equivalents) and N,N-diisopropylethylamine (1.31 ml, 6 equivalents). The medium was stirred at room temperature for 3 hours. The solvent was evaporated off under vacuum (P=9 mbar, T bath = 44°C). The crude product was taken up in 30 ml of distilled water and the aqueous phase is then extracted with 5 x 60 ml of dichloromethane. The organic phase was washed with saturated NaCl solution and then dried over magnesium sulfate. The solvent was evaporated off under vacuum. The crude reaction product was purified by flash chromatography on silica gel, eluting with a dichloromethane/methanol mixture(95/5 by volume). The 197 mg thus purified were dissolved in ethyl acetate (2 ml), followed by addition of 4.8N hydrochloric ethanol solution (17 µl) and the medium was stirred at room temperature for 18 hours. After concentration of the solvent followed by drying, 205 mg of 1-methylpyrrolidine-2-carboxylic acid {2-methoxy-5-[2-methyl-3-oxo-3-(3,4,5-trimethoxyphenyl)propenyl]-phenyl}amide were obtained, the characteristics of which were as follows:

- LC/MS (50x4.6 mm column of Hypersil BDS C18 3 µm silica; linear elution gradient from 5 to 90% acetonitrile, containing 0.05% trifluoroacetic acid, in water, also containing 0.05% trifluoroacetic acid, in 3.5 minutes at a flow rate of 1 mL/minute); retention time = 3.03 minutes.

- Mass spectrum: 486.32 ([M+H]⁺)

Example 13 : (S)-2-Amino-3-hydroxypropanoic acid {2-methoxy-5-[2-methyl-3-oxo-3-(3,4,5-trimethoxyphenyl)propenyl]phenyl}amide hydrochloride

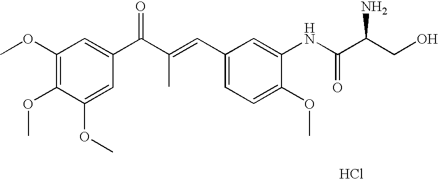

Step 1: 3-(3-Amino-4-methoxyphenyl)-2-methyl-1-(3,4,5-trimethoxyphenyl)propenone (700 mg, 1.96 mmol) was dissolved in 25 ml of dichloromethane, followed by addition of HOBT (298 mg, 1.2 equivalents), EDCI (422 mg, 1.2 equivalents) and N-Boc-L-serine (452 mg, 1.2 equivalents). The medium was stirred at room temperature for 3 days. 50 ml of dichloromethane and 25 ml of water were added. The organic phase was separated out after settling of the phases, washed with saturated NaCl solution and then dried over magnesium sulfate. The solvent was evaporated off under vacuum. The crude reaction product was purified by flash chromatography on silica gel, eluting with a dichloromethane/ethyl acetate mixture (7/3 by volume). 800 mg of (S)-2-Boc-amino-3-hydroxypropanoic acid {2-methoxy-5-[2-methyl-3-oxo-3-(3,4,5-trimethoxyphenyl)propenyl]phenyl}amide were thus obtained in the form of an orange-colored oil which was used without further modification in the following step, the characteristics of which product were as follows:

- Mass spectrum (EI) : m/z = 544

Step 2: (S)-2-Boc-amino-3-hydroxypropanoic acid {2-methoxy-5-[2-methyl-3-oxo-3-(3,4,5-trimethoxyphenyl)propenyl]}amide (800 mg) was dissolved in 2.5 ml of dioxane, followed by dropwise addition of 2.5 ml of a 4M solution of hydrochloric acid in dioxane. The reaction medium is stirred at room temperature for 20 hours; after concentration of the solvent under reduced pressure, the residue was taken up in 50 ml of water, brought to pHby addition of saturated aqueous sodium hydrogen carbonate solution and then extracted three times with 25 ml of dichloromethane. The combined organic phases were washed with water, dried over magnesium sulfate and concentrated under reduced pressure. The residue was purified by flash chromatography on silica gel, eluting with a dichloromethane/methanol mixture (95/5 by volume). 360 mg of (S)-2-amino-3-hydroxypropanoic acid {2-methoxy-5-[2-methyl-3-oxo-3-(3,4,5-trimethoxyphenyl)propenyl]phenyl}amide were thus obtained in the form of an amorphous beige-colored solid.

240 mg (0.54 mmol) of the base obtained above were dissolved in 5 ml of diisopropyl ether and 1 ml of methanol. 0.54 ml of a 1M solution of hydrochloric acid in diisopropyl ether was then added dropwise and the mixture is left to crystallize for three hours. After filtration followed by air-drying, 200 mg of (S)-2-amino-3-hydroxypropanoic acid {2-methoxy-5-[2-methyl-3-oxo-3-(3,4,5-trimethoxyphenyl)propenyl]phenyl}amide hydrochloride were obtained in the form of white crystals, the characteristics of which were as follows:

- Mass spectrum (EI) : m/z = 494

- Elemental analysis : %C = 56.93; %H = 6.58; %N = 5.61; %Cl = 7.12.

Example 14 : Aminoacetic acid {2-methoxy-5-[2-methyl-3-oxo-3-(3,4,5-trimethoxyphenyl)propenyl]phenyl}amide hydrochloride

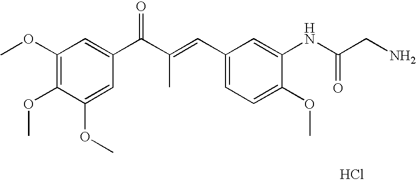

Step 1: 3-(3-Amino-4-methoxyphenyl)-2-methyl-1-(3,4,5-trimethoxyphenyl)propenone (175 mg, 0.5 mmol) was dissolved in 10 ml of dichloromethane, followed by addition of HOBT (75 mg, 1.1 equivalents), EDCI (106 mg, 1.1 equivalents) and N-Boc-glycine (96 mg, 1.1 equivalents). The medium was stirred at room temperature for 3 days. 10 ml of dichloromethane and 10 ml of water were added. The organic phase was separated out after settling of the phases, washed with saturated sodium chloride solution and then dried over magnesium sulfate. The solvent was evaporated off under vacuum. The crude reaction product was purified by flash chromatography on silica gel, eluting with a dichloromethane/ethyl acetate mixture (6/4 by volume). 250 mg of Boc-aminoacetic acid {2-methoxy-5-[2-methyl-3-oxo-3-(3,4,5-trimethoxyphenyl)propenyl]phenyl}amide were thus obtained in the form of a beige-colored foam which was used without further modification in the following step, the characteristics of which product were as follows:

- Mass spectrum (EI): m/z = 514

Step 2: 250 mg of Boc-aminoacetic acid {2-methoxy-5-[2-methyl-3-oxo-3-(3,4,5-trimethoxyphenyl)propenyl]phenyl}amide were dissolved in 2 ml of dioxane, followed by dropwise addition of 1.1 ml of a 4M solution of hydrochloric acid in dioxane. The reaction medium was stirred at room temperature for 20 hours. After concentration of the solvent under reduced pressure, the residue was dissolved in 5 ml of diisopropyl ether and 1 ml of methanol, and was then left to crystallize for 2 hours. After filtration followed by air-drying, 130 mg of aminoacetic acid {2-methoxy-5-[2-methyl-3-oxo-3-(3,4,5-trimethoxyphenyl)propenyl]phenyl}amide hydrochloride were obtained in the form of white crystals, the characteristics of which were as follows:

- Mass spectrum (EI): m/z = 450

- Elemental analysis: %C = 58.03; %H = 6.17; %N = 6.11; %Cl = 8.29.

Example 15 : (S)-2-Amino-3-methylbutanoic acid {2-methoxy-5-[2-methyl-3-oxo-3-(3,4,5-trimethoxy-phenyl)propenyl]phenyl}amide hydrochloride

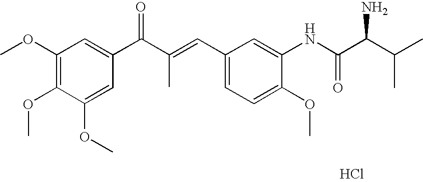

Step 1: 3-(3-Amino-4-methoxyphenyl)-2-methyl-1-(3,4,5-trimethoxyphenyl)propenone (176 mg, 0.5 mmol) was dissolved in 10 ml of dichloromethane, followed by addition of HOBT (75 mg, 1.1 equivalents), EDCI (1061.1 equivalents) and N-Boc-L-valine (120 mg, 1.1 equivalents). The medium was stirred at room temperature for 3 days. 15 ml of dichloromethane and 10of water were added. The organic phase was separated out after settling of the phases, washed with saturated sodium chloride solution and then dried over magnesium sulfate. The solvent was evaporated off under vacuum. The crude reaction product was purified by flash chromatography on silica gel, eluting with a dichloromethane/ethyl acetate mixture (8/2 by volume). 260 mg of (S)-2-Boc-amino-3-methylbutanoic acid {2-methoxy-5-[2-methyl-3-oxo-3-(3,4,5-trimethoxyphenyl)propenyl]phenyl}amide were thus obtained in the form of a beige-colored foam which was used without further modification in the following step, the characteristics of which product were as follows:

- Mass spectrum (EI): m/z = 556

Step 2: (S)-2-Boc-amino-3-methylbutanoic acid {2-methoxy-5-[2-methyl-3-oxo-3-(3,4,5-trimethoxyphenyl)propenyl]phenyl}amide (260 mg) was dissolved in 2 ml of dioxane, followed by dropwise addition of 1.05 ml of a 4M solution of hydrochloric acid in dioxane. The reaction medium was stirred at room temperature for 20 hours. After concentration of the solvent under reduced pressure, the residue was dissolved in 5 ml of diisopropyl ether and 0.5of methanol, and was then left to crystallize for 3 hours. After filtration followed by air-drying, 170 mg of (S)-2-amino-3-methylbutanoic acid {2-methoxy-5-[2-methyl-3-oxo-3-(3,4,5-trimethoxyphenyl)propenyl]phenyl}amide hydrochloride were obtained in the form of white crystals, the characteristics of which were as follows:

- Mass spectrum (EI): m/z = 493

- Elemental analysis: %C = 60.56; %H = 7.03; %N = 5.16; %Cl = 7.84.

Example 16 : E-2-Methyl-3-(1-methyl-1-H-indol-5-yl)-1-(3-methoxy-4,5-methylenedioxyphenyl)propenone

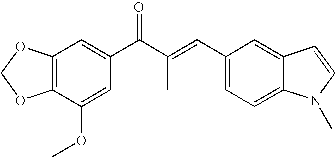

The process was performed as in step 1 of example 1, but starting with 2.1 g of 1-(3-methoxy-4,5-methylenedioxyphenyl)propanone - which may be prepared according to J. Org. Chem. 1981, 46(14), 2969-71 - and 3.18 g of 1-methylindole-5-carboxaldehyde - which may be prepared according to Terent"ew et al., J. Gen. Chem USSR (1962), 32, 1311 - in 100 mL of ethanol containing 2 mL of piperidine and 1 mL of acetic acid, by refluxing for 96 hours. After purification by flash chromatography on silica gel (70-230 mesh), eluting with a mixture of cyclohexane and ethyl acetate (80/20 by volume), followed by recrystallization from isopropanol, 1.05 g of pure E-2-methyl-3-(1-methyl-1-H-indol-5-yl)-1-(3-methoxy-4,5-methylenedioxyphenyl)propenone were obtained in the form of pale yellow crystals, the characteristics of which were as follows:

- melting point (Kofler) = 129°C.

- elemental analysis:% C = 72.10; %H = 5.28; % N = 4.06.

Example 17 : E-2-Methyl-3-(1-H-indol-5-yl)-1-(3-methoxy-4,5-methylenedioxyphenyl)propenone

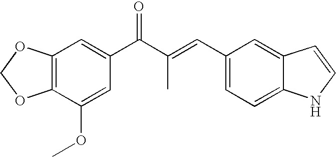

Step 1: 5 g of 5-indolecarboxaldehyde were dissolved in 90 ml of DMF and 18 ml of DMSO in a 250 ml three-necked flask under an argon atomosphere, and the mixture was then cooled to 0°C. 2.06 g of 60% sodium hydride in oil were then added portionwise and stirring was then continued while allowing the mixture to return to room temperature, until the evolution of gas had ceased. 8.6 g of (2-trimethylsilylethyl)oxymethyl chloride were then added dropwise and the mixture was then stirred for 20 hours at room temperature. The reaction medium was then poured into a mixture of 300 ml of water and 100 g of crushed ice, and then extracted 3 times with 150 ml of ethyl acetate. The combined organic phases were washed with saturated aqueous sodium chloride solution, dried over magnesium sulfate and concentrated to dryness under reduced pressure. The brown oil obtained was purified by flash chromatography on silica gel (70-230 mesh), eluting with a mixture of cyclohexane and ethyl acetate (70/30 by volume) to give 9 g of 1-(2-trimethylsilylethyl)oxymethylindole-5-carboxaldehyde in the form of an orange-colored oil, which was used without further modification in the following step.

Step 2: The process was performed as in step 1 of example 1, but starting with 2.1 g of 1-(3-methoxy-4,5-methylenedioxyphenyl)propanone - which may be prepared according to J. Org. Chem. 1981, 46(14), 2969-71 - and 2.76 g of 1-(2-trimethylsilylethyl)oxymethylindole-5-carboxaldehyde in 100 mL of ethanol containing 2 mL of piperidine and 1 mL of acetic acid, by refluxing for 96 hours. After purification by flash chromatography on silica gel (70-230 mesh), eluting with a mixture of cyclohexane and ethyl acetate (70/30 by volume), followed by crystallization from diisopropyl ether, 2 g of pure E-2-methyl-3-[1-(2-trimethylsilylethyl)oxymethyl-1-H-indol-5-yl]-1-(3-methoxy-4,5-methylenedioxyphenyl)propenone were obtained in the form of white crystals, the characteristics of which were as follows:

- melting point (Kofler) = 90°C

Step 3: 2 g of E-2-methyl-3-[1-(2-trimethylsilylethyl)oxymethyl-1-H-indol-5-yl]-1-(3-methoxy-4,5-methylenedioxyphenyl)propenone were dissolved in 42 ml of THF, followed by addition of 4.3 ml of a 1M solution of tetra-N-butylammonium fluoride in THF, and the mixture was refluxed for 20 hours. After concentration under reduced pressure, the reaction medium was taken up in 75 ml of ethyl acetate and 75 ml of water. The organic phase was separated out after settling of the phases, washed with water, dried over magnesium sulfate and concentrated to dryness under reduced pressure. The red oil obtained was purified by flash chromatography on silica gel (70-230 mesh), eluting with a mixture of cyclohexane and ethyl acetate (70/30 by volume), followed by recrystallization from isopropanol, to give 420 mg of pure E-2-methyl-3-(1-H-indol-5-yl)-1-(3-methoxy-4,5-methylenedioxyphenyl)propenone in the form of a beige-colored solid, the characteristics of which were as follows:

- melting point (Kofler) = 140°C

Example 18 : E-2-Methyl-3-[1-(2-dimethylaminoethyl)-1-H-indol-5-yl]-1-(3-methoxy-4,5-methylenedioxyphenyl)propenone

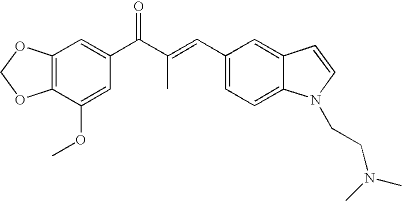

336 mg of E-2-methyl-3-(1-H-indol-5-yl)-1-(3-methoxy-4,5-methylenedioxyphenyl)propenone, obtained in example 17, were dissolved in 10.5 ml of pyridine in a 250 ml three-necked flask under an argon atmosphere, the solution was then cooled to 0°C and 90 mg of 60% sodium hydride in oil were added. After stirring for 1 hour at room temperature, the mixture was again cooled to 0°C and 144 mg of (2-chloroethyl)dimethylamine hyrochloride were added dropwise. The mixture was then maintained at 60°C for 5 hours. After concentration under reduced pressure, the reaction medium was taken up in 50of ethyl acetate, washed with water, dried over magnesium sulfate and concentrated to dryness under reduced pressure. The yellow-brown foam obtained was purified by flash chromatography on silica gel (70-230 mesh), eluting with a mixture of methanol and dichloromethane (2/98 by volume), followed by crystallization from diisopropyl ether, to give 400 mg of pure E-2-methyl-3-[1-(2-dimethylaminoethyl)-1-H-indol-5-yl]-1-(3-methoxy-4,5-methylenedioxyphenyl)propenone in the form of a beige-colored solid, the characteristics of which were as follows:

- melting point (Kofler) = 118°C

Example 19 : E-2-Methyl-3-(1-H-indol-5-yl)-1-(3,4,5-trimethoxyphenyl)propenone

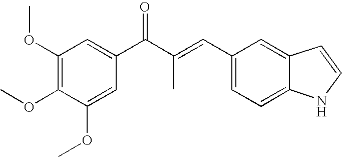

Step 1 : The process was performed as in step 1 of example 1, but starting with 2.24 g of 1-(3,4,5-trimethoxyphenyl)propanone and 2.76 g of 1-tert-butyloxycarbonylindole-5-carboxaldehyde - which may be prepared according to J. Org. Chem. 2002, 67(17), 6256-59 - in 100 mL of ethanol containing 2 mL of piperidine and 1 mL of acetic acid, by refluxing for 48 hours. After purification by flash chromatography on silica gel (70-230 mesh), eluting with a mixture of cyclohexane and ethyl acetate (70/30 by volume), 2.2 g of pure E-2-methyl-3-[1-(1-tert-butyloxycarbonyl-1-H-indol-5-yl]-1-(3,4,5-trimethoxyphenyl)propenone were obtained in the form of a pale yellow oil, which was used without further modification in the following step.

Step 2: 0.7 g of E-2-methyl-3-[1-(1-tert-butyloxycarbonyl-1-H-indol-5-yl]-1-(3,4,5-trimethoxyphenyl)propenone was dissolved in 15 ml of THF. 1.5 ml of methanol and 0.25 g of sodium methoxide were then added successively and the mixture was then stirred for 18 hours at room temperature. After concentration under reduced pressure, the reaction medium was taken up in 75 ml of ethyl acetate and 35 ml of water. The organic phase was separated out after settling of the phases, washed with water, dried over magnesium sulfate and concentrated under reduced pressure. After purification by flash chromatography on silica gel (70-230 mesh), eluting with a mixture of dichloromethane and diisopropyl ether (50/50 by volume), 505 mg of pure E-2-methyl-3-(1-H-indol-5-yl)-1-(3,4,5-trimethoxyphenyl)propenone were obtained in the form of an orange-colored oil, the characteristics of which were as follows:

- mass spectrum (EI):m/z = 351.

- elemental analysis:% C = 71.26; %H = 6.54; % N = 3.72.

Example 20 : E-2-Methyl-3-[1-(2-dimethylaminoethyl)-1-H-indol-5-yl]-1-(3,4,5-trimethoxyphenyl)propenone hydrochloride

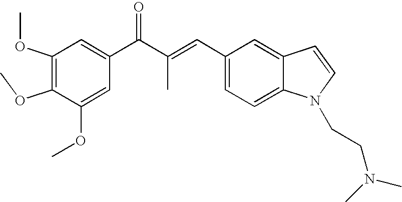

The process was performed as in example 18, but starting with 350 mg of 2-methyl-3-(1-H-indol-5-yl)-1-(3,4,5-trimethoxyphenyl)propenone, obtained in example 19, 90 mg of 60% sodium hydride in oil and 143 mg of (2-chloroethyl)dimethylamine hydrochloride in 10 ml of pyridine. After purification by flash chromatography on silica gel (70-230 mesh), eluting with a mixture of ethanol and dichloromethane (5/95 by volume), 150 mg of pure E-2-methyl-3-[1-(2-dimethylaminoethyl)-1-H-indol-5-yl]-1-(3,4,5-trimethoxyphenyl)propenone in the form of a yellow oil. This oil was redissolved in 2.5of diethyl ether, followed by addition of 0.4 ml of a 1M solution of hydrochloric acid in diethyl ether, and the mixture was left to crystallize for 20 hours. After filtration, washing with diethyl ether and drying under reduced pressure in the presence of phosphorus pentoxide, 120 mg of E-2-methyl-3-[1-(2-dimethylaminoethyl)-1-H-indol-5-yl]-1-(3,4,5-trimethoxyphenyl)propenone hydrochloride were obtained in the form of pink-beige crystals, the characteristics of which were as follows:

- melting point (Kofler) = 127°C

- elemental analysis: % C = 65.17; %H = 7.09; % N = 5.68; %Cl = 7.88.

Example 21 : E-2-Methyl-3-(1-hydroxymethyl-1-H-indol-5-yl)-1-(3,4,5-trimethoxyphenyl)propenone

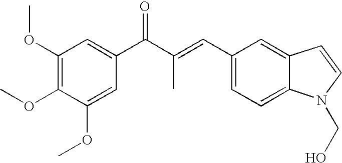

2.5 ml of an aqueous 37% formaldehyde solution and then 0.55 ml of aqueous 1N sodium hydroxide solution were successively added to a solution of 176 mg of E-2-methyl-3-(1-H-indol-5-yl)-1-(3,4,5-trimethoxyphenyl)propenone, obtained in example 19, in 3 ml of ethanol. After stirring for 20 hours at room temperature, 25 ml of water were added to the suspension obtained, and the mixture was extracted 3 times with 15 ml of ethyl acetate. The combined organic phases were washed with water, dried over magnesium sulfate and concentrated under reduced pressure. After purification by recrystallization from ethyl acetate, 35 mg of pure E-2-methyl-3-(1-hydroxymethyl-1-H-indol-5-yl)-1-(3,4,5-trimethoxyphenyl)propenone were obtained in the form of beige-colored crystals, the characteristics of which were as follows:

- melting point (Kofler) = 185°C

- elemental analysis: % C = 69.13; %H = 6.14; % N = 3.36.

BIOLOGICAL RESULTS

| | | | | Percentage of detachment of | | | |
|---|---|---|---|---|---|---|---|
| | Tubulin: | Inhibition of | | HDMEC cells induced with the | | | |
| | inhibition of | cell proliferation | | compound cited in the | | | C51 colon |
| Example | polymerization | IC_{50 (μM)} | | example at a concentration of: | | | tumor necrosis |
| N° | IC_{50 (μM)} | HDMEC | HeLa | 1 μM | 0.3 μM | 0.1 μM | in vivo |
| 1 | 0.3 | 0.0025- | 0.0036 @ | 37 @ 42 | 18 @ 38 | 21 @ 36 | Grade 5 at |
| | | 0.0032 | 0.0043 | | | | 24.5 mg/kg |
| 2 | 2.5 | 0.0019- | 0.039 @ | 31 @ 49 | 17 @ 32 | 16 @ 21 | Grade 5 at |
| | | 0.0040 | 0.104 | | | | 50 mg/kg |
| 3 | 0.5 | <0.019 | <0.019 | 34 | 37 | 31 | nd |
| 4 | 0.6 | nd | nd | nd | nd | nd | nd |
| 5 | 0.7 | 0.03 | 0.005- | 32 | nd | nd | Grade 5 at |
| | | | 0.01 | | | | 25 mg/kg |
| 6 | 0.8 | 0.017- | nd | 28 | nd | nd | Grade 5 at |
| | | 0.02 | | | | | 50 mg/kg |
| 7 | 2.0 | 0.3-0.5 | nd | nd | nd | nd | nd |
| 8 | 0.8 | nd | nd | nd | nd | nd | nd |
| 9 | 3.5 | nd | nd | nd | nd | nd | nd |
| 10 | np | 0.207- | nd | 36 | nd | nd | Grade 5 at |
| | | 0.225 | | | | | 25 mg/kg |
| 11 | np | 0.4 | nd | nd | nd | nd | nd |
| 12 | nr | 0.32 | nd | nd | nd | nd | nd |
| 13 | 10 (np) | 0.03 | 0.1 | 31 | nd | nd | Grade 5 at |
| | | | | | | | 12.5 mg/kg |
| 14 | 25 (np) | 0.01 | nd | 36 | nd | nd | Grade 5 at |
| | | | | | | | 25 mg/kg |

| | | | | | | | |
|---|---|---|---|---|---|---|---|
| 15 | 9 (np) | 0.03-0.06 | nd | nd | nd | nd | nd |
| 16 | 0.5 | nd | nd | nd | nd | nd | nd |
| 17 | 0.8 | nd | nd | nd | nd | nd | nd |
| 18 | 2.0 | nd | nd | nd | nd | nd | nd |
| 19 | 0.8 | nd | nd | nd | nd | nd | nd |
| 20 | 2.0 | nd | nd | nd | nd | nd | nd |
| 21 | 0.5 | nd | nd | nd | nd | nd | nd |

nd: not determined
np: not pertinent

What is Claimed is:

1.A product corresponding to formula (I) below: 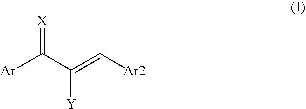
in which:
a)Y is selected from halogen, C₁-C₇ linear alkyl, C₁-C₇ branched alkyl, substituted C₁-C₇ linear alkyl, substituted C₁-C₇ branched alkyl, cycloalkyl, substituted cycloalkyl, NH₂, NH(R4), N(R4)₂, aralkyl, substituted aralkyl, COOH, COO(R4), CONH₂, CONH(R4), CON(R4)₂, and CN, in which R4 represents an optionally substituted C₁-C₇ alkyl or cycloalkyl group and, when two radicals R4 are present, they are optionally linked together to form a ring;
b)Ar2 is selected from: 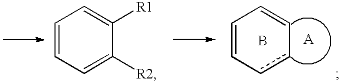
in which:
1)when Ar2 is 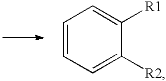
then one of the radicals R1 and R2 is selected from NH₂, NH₂·HZ, NHC(O)-amino acid, NH-(GP); N=(GP); in which GP is a metabolizable substituent allowing the functional group to be changed:
NH-(GP) → NH₂orN=(GP) → NH₂
and in which HZ is an organic or mineral acid; and
the other radical R1 or R2 is selected from CH₃, C₂H₅, OCH₃, OC₂H₅, SCH₃, NH(R5), N(R5)₂, N(R5)(GP), and N(R5)C(O)-amino acid, in which R5 represents a C₁-C₂ alkyl group and, when two radicals R5 are present, they are optionally linked together to form a ring;
2)when Ar2 is 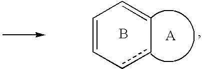
then A is a 5- or 6-membered heterocycle, fused to a benzene ring B, said heterocycle A is aromatic or non-aromatic, comprising one or two hetero atoms, at least one of which is a nitrogen atom linked directly to B and bearing a side chain R8, in which R8 is chosen from H, (C₁-C₃)alkyl, (C₁-C₃)alkyl-OH, (C₁-C₃)alkyl-O(C₁-C₃)alkyl, (C₁-C₃)alkyl-NH₂, (C₁-C₃)alkyl-NH(R7), (C₁-C₃)alkyl-N(R7)₂, 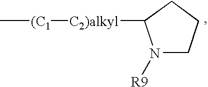
in which R9 is chosen from H and (C₁-C₃)alkyl, in which each R7 independently represents a (C₁-C₃)alkyl or (C₃-C₇) cycloalkyl group, or, when two radicals R7 are present, the two radicals R7 are optionally linked together to form a 5-membered heterocycle;
c)X is selected from O, NOH, and NO(R3), in which R3 is selected from H, C₁-C₇ linear alkyl, C₁-C₇ branched alkyl, cycloalkyl, C₁-C₇ linear haloalkyl, C₁-C₇ branched haloalkyl, substituted cycloalkyl, halocycloalkyl, aralkyl, and substituted aralkyl; and
d)Ar is selected from 2,5-dimethoxyphenyl, 2,3,4-trimethoxyphenyl, 3,4,5-trimethoxyphenyl, 2,3,5-trimethoxyphenyl, 2,4,5-trimethoxyphenyl, 2,3,4,5-tetramethoxyphenyl, 3-methoxy-4,5-methylenedioxyphenyl, 3-methoxy-4,5-ethylenedioxyphenyl, 2-methoxy-4,5-methylenedioxyphenyl, 2-methoxy-4,5-ethylenedioxyphenyl, 2-methoxy-3,4-methylenedioxyphenyl, and 2-methoxy-3,4-ethylenedioxyphenyl radicals.

2.The product as claimed in claim 1, wherein Ar2 is 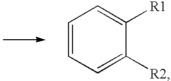
wherein one of the radicals R1 and R2 is NHC(O)-amino acid, and wherein the amino acid is serine.

3.The product as claimed in claimwherein X is oxygen.

4.The product as claimed in any one of claims 1 to 3, wherein Ar is 3,4,5-trimethoxyphenyl or 3-methoxy-4,5-methylenedioxyphenyl.

5.The product as claimed in any one of claims 1 to 3, wherein Y is selected from Cl, Br, CH₃, and CH₂CH₃.

6.The product as claimed in any one of claims 1 to 3,wherein Ar2 is

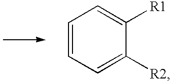

7.The product as claimed in claim 6, wherein R1 and R2 are selected from combinations (R1, R2) consisting of, respectively, (NH₂, OCH₃), (NH₂, OC₂H₅), (NH₂, N(R5)₂), (N(R5)₂, OCH₃), (N(R5)₂, OC₂H₅), (N(R5)₂, NH₂), (OCH₃, NH₂), and (OC₂H₅, NH₂).

8.The product as claimed in any one of claims 1 to 3, wherein Ar2 is

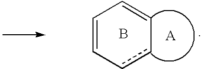

9.The product as claimed in claim 8, wherein Ar2 is selected from

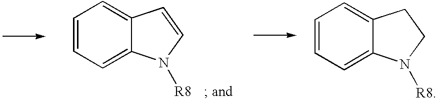

10.The product as claimed in claim 9, wherein R8 is chosen from methyl, hydroxymethyl, and 2-dimethylaminoethyl groups.

11.The product as claimed in claim 1, wherein the product is chosen from:
E-3-(3-Amino-4-methoxyphenyl)-2-methyl-1-(3,4,5-trimethoxyphenyl)propenone;
E-3-(4-Amino-3-methoxyphenyl)-2-methyl-1-(3,4,5-trimethoxyphenyl)propenone;
E-3-(3-Amino-4-methoxyphenyl)-2-methyl-1-(2,5-dimethoxyphenyl)propenone hydrochloride;
E-3-(3-Amino-4-methoxyphenyl)-2-bromo-1-(3,4,5-trimethoxyphenyl)propenone;
E-2-Methyl-3-(1-methyl-1-H-indol-5-yl)-1-(3,4,5-trimethoxyphenyl)propenone;
E-2-Methyl-3-(1-methyl-2,3-dihydro-1-H-indol-5-yl)-1-(3,4,5-trimethoxyphenyl)propenone;
E-2-Methyl-3-(1-methyl-1-H-indol-5-yl)-1-(3,4,5-trimethoxyphenyl)propenoneoxime;
E-2-Methyl-3-[1-(2-dimethylaminoethyl)-1-H-indol-5-yl]-1-(3,4,5-trimethoxyphenyl)propenone hydrochloride;
E-2-Methyl-3-(1-hydroxymethyl-1-H-indol-5-yl)-1-(3,4,5-trimethoxyphenyl)propenone;
E-2-Methyl-3-(1-methyl-1-H-indol-5-yl)-1-(3-methoxy-4,5-methylenedioxyphenyl)propenone;
E-2-Methyl-3-[1-(2-dimethylaminoethyl)-1-H-indol-5-yl]-1-(3-methoxy-4,5-methylenedioxyphenyl)propenone;
(S)-2,6-Diaminohexanoic acid {2-methoxy-5-[2-methyl-3-oxo-3-(3,4,5-trimethoxyphenyl)propenyl]phenyl}amide dihydrochloride;
3-(3-[N-w-nitro-L-arginineamido]-4-methoxyphenyl)-2-methyl-1-(3,4,5-trimethoxyphenyl)propenone;
Aminoacetic acid {2-methoxy-5-[2-methyl-3-oxo-3-(3,4,5-trimethoxyphenyl)propenyl]phenyl}amide hydrochloride;
(S)-2-amino-3-hydroxypropanoic acid {2-methoxy-5-[2-methyl-3-oxo-3-(3,4,5-trimethoxyphenyl)propenyl]phenyl}amide hydrochloride; and
1-methylpyrrolidine-2-carboxylic acid {2-methoxy-5-[2-methyl-3-oxo-3-(3,4,5-trimethoxyphenyl)propenyl]-phenyl}amide hydrochloride.

12.The product as claimed in any one of claims 1 to 3, wherein one of the radicals R1 and R2 is NHC(O)-amino acid, and wherein the amino acid is selected from natural amino acids and unnatural amino acids.

13.The product as claimed in claim 12, wherein the amino acid is chosen from glycine, lysine, N-methylproline, serine, and N-ω-nitroarginine, and wherein the amino acid is in enantiomerically pure or racemic form, or is enriched in an enantiomer.

14.The product as claimed in any one of claims 1 to 3, wherein the product is in free or salified form.

15.The product as claimed in claim 14, wherein the product is in salified form.

16.The product as claimed in claim 15, wherein the salified form is a hydrochloride.

17.The product as claimed in claim 13, wherein the product is (S)-2-amino-3-hydroxypropanoic acid {2-methoxy-5-[2-methyl-3-oxo-3-(3,4,5-trimethoxyphenyl)propenyl]phenyl}amide hydrochloride.

18.A pharmaceutical composition comprising a product as claimed in any one of claims 1 to 3, in combination with a pharmaceutically acceptable excipient.

19.A pharmaceutical composition comprising a product as claimed in claim 16, in combination with a pharmaceutically acceptable excipient.

20.A method for inhibiting tubulin polymerization, comprising administering to a host in need of such treatment a pharmaceutically effective amount of the product as claimed in any one of claims 1 to 3.

21.A method for promoting detachment of endothelial cells forming a wall of a blood vessel supplying a tumor, comprising administering to a host in need of such treatment a pharmaceutically effective amount of the product as claimed in any one of claims 1 to 3.

22.A method for promoting tumor necrosis, comprising administering to a host in need of such treatment a pharmaceutically effective amount of the product as claimed in any one of claims 1 to 3.

23.A method for treating a pathological condition, comprising administering to a host in need of such treatment a pharmaceutically effective amount of the product as claimed in any one of claims 1 to 3.

24.The method as claimed in claim 23, wherein the pathological condition is cancer.
